(19) United States
(12) Patent Application Publication
(10) Pub. No.: US 2015/0132344 A1
(43) Pub. Date: May 14, 2015

(54) Oral Leptin Formulations and Uses Thereof

(71) Applicants: Moise Bendayan, Saint-Laurent (CA); Philippe Cammisotto, Lasalle (CA)

(72) Inventors: Moise Bendayan, Saint-Laurent (CA); Philippe Cammisotto, Lasalle (CA)

(73) Assignee: UNIVERSITE DE MONTREAL, Montreal, QC (CA)

(21) Appl. No.: 14/357,893

(22) PCT Filed: Nov. 18, 2011
(86) PCT No.: PCT/CA2011/050720

§ 371 Date: Jan. 21, 2015

(51) Int. Cl. A61K 38/22; A61K 9/00; A61K 31/575; A61K 45/06; A61K 38/57; A61K 33/00

(52) U.S. Cl. CPC A61K 38/2264; A61K 38/57; A61K 33/00; A61K 31/575; A61K 45/06; A61K 9/0053
USPC 424/400; 514/5.8; 424/717; 514/171; 514/5.3

(57) ABSTRACT

An oral combination therapy or oral composition that protects orally administered exogenous leptin from the environment of the gastrointestinal tract, allowing delivery of the exogenous leptin to the bloodstream. The oral combination therapy includes (a) leptin or a leptin functional derivative; (b) a stomach acid-neutralizing agent such as a buffer; and (c) a pancreatic protease inhibitor that protects the exogenous leptin from degradation by pancreatic enzymes; and (d) a bile acid or a bile acid analog that facilitates absorption of the exogenous leptin. The oral combination therapy or oral composition may also include at least one agent that stimulates endogenous leptin secretion; as well as at least one agent capable of promoting, enhancing or improving adherence to treatment such as a sweetener or a satiety triggering agent. The oral combination therapy or oral composition can be a single composition in liquid or solid form, or can be administered simultaneously or sequentially so that the components mix in the stomach cavity of the patient. Methods and uses relating to the above oral combination therapy or oral composition to treat or prevent diseases of conditions that are associated with or can be ameliorated by leptin (e.g., obesity, weight gain, diabetes) are also included.

### CROSS REFERENCE TO RELATED APPLICATIONS

This application is a PCT application no. CA2011/* filed on Nov. 18, 2011 and published in English under PCT Article 21(2), which itself claims benefit of U.S. provisional application Ser. No. 61/415,095, filed on Nov. 18, 2010. All documents above are incorporated herein in their entirety by reference.

### FIELD OF THE INVENTION

The present invention relates to the peptide hormone leptin. More specifically, the present invention is concerned with a formulation or combination therapy allowing the effective oral administration of leptin.

### REFERENCE TO SEQUENCE LISTING

Pursuant to 37 C.F.R. 1.821(c), a sequence listing is submitted herewith as an ASCII compliant text file named 12810_{—}427_ST25, created on Nov. 18, 2011 and having a size of 145 kilobytes. The content of the aforementioned file is hereby incorporated by reference in its entirety.

### BACKGROUND OF THE INVENTION

Unhealthy lifestyles associated with excessive caloric intake and lack of physical activity are an increasing problem. In many instances, low-calorie diets have proved inefficient to cure obesity since many obese patients quickly re-gain the weight they lost by not being able to maintain healthy eating habits when faced with increased hunger.

A significant advance in understanding the regulation of food intake was the discovery of the polypeptide hormone leptin. Leptin is encoded by the obese (ob) gene and plays a fundamental role in, for example, controlling appetite and regulating energy expenditure. Human leptin is initially translated as a 167 amino acid polypeptide which includes an amino-terminal secretory signal sequence of 21 amino acids. The signal sequence is removed following translocation of the polypeptide into rough endoplasmic reticulum, resulting in a mature non-glycosylated leptin polypeptide of 146 amino acids having a molecular weight of approximately 16 kDa.

Leptin alone is quite unstable in circulation and has a short half-life in its unprotected or unbound form. Thus, physiological leptin is found coupled to a binding protein (e.g., a soluble receptor) which protects it from degradation and increases its half-life. Leptin is synthesized by white adipose tissue (Zhang et al., 1994) and by chief cells of the gastric glands lining the lumen of the lower stomach, which store the hormone in their secretory granules in its complexed form (Cinti et al., 2000; Cammisotto et al., 2005, 2010a; Sobhani et al., 2000).

In the stomach, leptin is secreted complexed to a protective binding protein that results from the cleavage of membrane-bound leptin receptor. The cleavage of the membrane bound leptin receptor generates the soluble isoform of this receptor. Upon appropriate stimulation (e.g., the intake of food), complexed leptin is secreted into the gastric juice and eventually reaches the duodenum where it binds to leptin receptors present on the luminal membrane of enterocytes (Cammisotto et al., 2006; Cammisotto et al., 2010b, Guilmeau et al., 2003). A significant fraction of leptin is then internalized by the enterocytes and eventually delivered to the bloodstream in its intact form (Cammisotto et al., 2007, 2010b). Once in circulation, complexed leptin can reach the central nervous system via a specific transendothelial carrier or receptor located at the level of the blood-brain barrier. The binding of leptin to its hypothalamic receptors is thought to be fundamental for the proper control of appetite and energy storage (Campfield et al., 1995).

The present invention seeks to provide a new method for the oral administration of leptin.

The present description refers to a number of documents, the content of which is herein incorporated by reference in their entirety.

### SUMMARY OF THE INVENTION

The present invention relates to the surprising discovery that orally administered exogenous leptin can be formulated (in the absence of its natural protective binding protein) to cross the intestinal epithelium, be delivered in its active form to the bloodstream, and act on hypothalamic cells to regulate appetite and/or metabolism.

In one aspect, the present invention relates to the use of: (a) leptin or a leptin functional derivative; (b) a stomach acid neutralizing agent; (c) a pancreatic protease inhibitor; and (d) a bile acid or a bile acid analog; for orally delivering the leptin or leptin functional derivative to a subject's bloodstream in an active form thereof, or for the manufacture of an oral combination therapy for same.

In an embodiment, above mentioned leptin or functional derivative thereof is: a leptin variant; a leptin analog; a leptin prodrug; or any combination thereof. In another embodiment. In another embodiment, the above mentioned leptin or functional derivative thereof is recombinant leptin. In another embodiment, the above mentioned leptin or functional derivative thereof is human leptin.

In another embodiment, the above mentioned stomach acid neutralizing agent comprises a buffer. In another embodiment, the above mentioned buffer is a phosphate buffer; a bicarbonate buffer; a citrate buffer; an acetate buffer; or any combination thereof. In another embodiment, the above mentioned stomach acid neutralizing agent is present in an amount to inhibit the digestion of the leptin or leptin functional derivative by gastric pepsin in the subject.

In another embodiment, the above mentioned the pancreatic protease inhibitor comprises: a trypsin inhibitor; a chymotrypsin inhibitor; a carboxypeptidase inhibitor; an elastase inhibitor; or any combination thereof. In another embodiment, the above mentioned pancreatic protease inhibitor is present in an amount to inhibit the digestion of the leptin or leptin functional derivative by one or more pancreatic proteases in the subject. In another embodiment, the above mentioned pancreatic protease inhibitor is aprotinin.

In another embodiment, the above mentioned bile acid or bile acid analog comprises: deoxycholic acid; cholic acid; chenodeoxycholic acid; taurocholic acid; taurochenodeoxycholic acid; glycocholic acid; glycochenocholic acid; 3β-monohydroxychloric acid; lithocholic acid; 3-hydroxy-12-ketocholic acid; 12-3-dihydrocholic acid; ursodesoxycholic acid; or an analog thereof; or any combination thereof. In another embodiment, the above mentioned bile acid or bile acid analog is: deoxycholic acid; taurocholic acid; lithocholic acid; an analog thereof; or any combination thereof. In another embodiment, the above mentioned bile acid or bile acid analog is present in an amount to allow intestinal absorption of the leptin or leptin functional derivative in the subject.

In another embodiment, the above mentioned use further comprises a sweetener.

In another embodiment, the above mentioned use further comprises a stimulator of endogenous leptin secretion or a satiety triggering agent. In another embodiment, the above mentioned stimulator of leptin secretion or satiety triggering agent is: glutamine; insulin: secretin; cholecystokinin (CCK); pentagastrin; a glucocorticoid; transretinoic acids; an analog thereof; or any combination thereof.

In another embodiment, the above mentioned stomach acid neutralizing agent is present at a concentration from about 10 mM to about 250 mM. In another embodiment, the above mentioned bile acid or bile acid analog is present at a concentration from about 1 mg/mL to about 25 mg/mL.

In another embodiment, one or more of compounds (a)-(d) mentioned above is in the form of: a tablet; a pill; a powder; a syrup; a liquid; a food; a dragee; a confectionary; or any combination thereof.

In another embodiment, the above mentioned oral combination therapy is an oral composition comprising (a)-(d). In another embodiment, all of compounds of (a)-(d), or the oral combination therapy, is eligible for natural health product status.

In another embodiment, the above mentioned use is for preventing, treating and/or managing a disease, condition or phenotype that is associated with low plasma leptin levels or that can be ameliorated by increasing plasma leptin levels; or for the manufacture of an oral combination therapy for same. In another embodiment, the above mentioned disease, condition or phenotype is: obesity, type 1 diabetes, type 2 diabetes, hypothalamic amenorrhea, cardiovascular diseases, depression, a hypoleptinemic disease, a leptin deficient state, weight gain, or a condition that can be ameliorated by weight loss or by an increase in the levels of plasma leptin.

In another aspect, the present invention relates to an oral combination therapy comprising: (a) leptin or a leptin functional derivative; (b) a stomach acid neutralizing agent; (c) a pancreatic protease inhibitor; and (d) a bile acid or a bile acid analog; for orally delivering the leptin or leptin functional derivative to a subject's bloodstream in an active form thereof.

In another embodiment, the above mentioned leptin or functional derivative thereof is: a leptin variant; a leptin analog; a leptin prodrug; or any combination thereof. In another embodiment, the above mentioned leptin or functional derivative thereof is recombinant leptin. In another embodiment, the above mentioned the leptin or functional derivative thereof is human leptin.

In another embodiment, the above mentioned stomach acid neutralizing agent comprises a buffer. In another embodiment, the above mentioned buffer is a phosphate buffer; a bicarbonate buffer; a citrate buffer; an acetate buffer; or any combination thereof. In another embodiment, the above mentioned stomach acid neutralizing agent is present in an amount to inhibit the digestion of the leptin or leptin functional derivative by gastric pepsin in the subject.

In another embodiment, the above mentioned pancreatic protease inhibitor comprises: a trypsin inhibitor; a chymotrypsin inhibitor; a carboxypeptidase inhibitor; an elastase inhibitor; or any combination thereof. In another embodiment, the above mentioned pancreatic protease inhibitor is present in an amount to inhibit the digestion of the leptin or leptin functional derivative by one or more pancreatic proteases in the subject. In another embodiment, the above mentioned pancreatic protease inhibitor is aprotinin.

In another embodiment, the above mentioned bile acid or bile acid analog comprises: deoxycholic acid; cholic acid; chenodeoxycholic acid; taurocholic acid; taurochenodeoxycholic acid; glycocholic acid; glycochenocholic acid; 3β-monohydroxychloric acid; lithocholic acid; 3-hydroxy-12-ketocholic acid; 12-3-dihydrocholic acid; ursodesoxycholic acid; or an analog thereof; or any combination thereof. In another embodiment, the above mentioned bile acid or bile acid analog is: deoxycholic acid; taurocholic acid; lithocholic acid; an analog thereof; or any combination thereof. In another embodiment, the above mentioned bile acid or bile acid analog is present in an amount to allow intestinal absorption of the leptin or leptin functional derivative in the subject.

In another embodiment, the above mentioned oral combination therapy further comprises a sweetener. In another embodiment, the above mentioned oral combination therapy further comprises a stimulator of endogenous leptin secretion or a satiety triggering agent. In another embodiment, the above mentioned stimulator of leptin secretion or satiety triggering agent is: glutamine; insulin: secretin; cholecystokinin (CCK); pentagastrin; a glucocorticoid; transretinoic acids; an analog thereof; or any combination thereof.

In another embodiment, the above mentioned stomach acid neutralizing agent is present at a concentration from about 10 mM to about 250 mM.

In another embodiment, the above mentioned bile acid or bile acid analog is present at a concentration from about 1 mg/mL to about 25 mg/mL.

In another embodiment, one or more of (a)-(d) comprised in the above mentioned oral combination therapy is in the form of: a tablet; a pill; a powder; a syrup; a liquid; a food; a dragee; a confectionary; or any combination thereof. In another embodiment, the above mentioned oral combination therapy is an oral composition comprising (a)-(d).

In another embodiment, the above mentioned oral combination therapy is eligible for natural health product status.

In another embodiment, the above mentioned oral combination therapy is for preventing, treating and/or managing a disease, condition or phenotype that is associated with low plasma leptin levels or that can be ameliorated by increasing plasma leptin levels; or for the manufacture of an oral combination therapy for accomplishing same. In another embodiment, the above mentioned disease, condition or phenotype is: obesity, type 1 diabetes, type 2 diabetes, hypothalamic amenorrhea, cardiovascular diseases, depression, a hypoleptinemic disease, a leptin deficient state, weight gain, or a condition that can be ameliorated by weight loss or by an increase in the levels of plasma leptin.

In another aspect, the present invention relates to a method for the oral administration of leptin, the method comprising administering to a subject a therapeutically effective amount of the oral combination therapy as defined above, wherein the leptin or leptin functional derivative is delivered to the subject's bloodstream in an active form thereof. In an embodiment, the above mentioned method is for preventing, treating and/or managing a disease, condition or phenotype that is associated with low plasma leptin levels or that can be ameliorated by increasing plasma leptin levels; or for the manufacture of an oral combination therapy for same. In another embodiment, the above mentioned disease, condition or phenotype is: obesity, type 1 diabetes, type 2 diabetes, hypothalamic amenorrhea, cardiovascular diseases, depression, a hypoleptinemic disease, a leptin deficient state, weight gain, or a condition that can be ameliorated by weight loss or by an increase in the levels of plasma leptin.

In another aspect, the present invention relates to an oral composition comprising: (a) leptin or a leptin functional derivative; (b) a stomach acid neutralizing agent; (c) a pancreatic protease inhibitor; and (d) a bile acid or a bile acid analog.

In another embodiment, the above mentioned leptin or functional derivative thereof is: a leptin variant; a leptin analog; a leptin prodrug; or any combination thereof. In another embodiment, the above mentioned leptin or functional derivative thereof is recombinant leptin. In another embodiment, the above mentioned leptin or functional derivative thereof is human leptin.

In another embodiment, the above mentioned stomach acid neutralizing agent comprises a buffer. In another embodiment, the above mentioned buffer is a phosphate buffer; a bicarbonate buffer; a citrate buffer; an acetate buffer; or any combination thereof. In another embodiment, the above mentioned stomach acid neutralizing agent is present in an amount to inhibit the digestion of the leptin or leptin functional derivative by gastric pepsin in the subject.

In another embodiment, the above mentioned pancreatic protease inhibitor comprises: a trypsin inhibitor; a chymotrypsin inhibitor; a carboxypeptidase inhibitor; an elastase inhibitor; or any combination thereof. In another embodiment, the above mentioned pancreatic protease inhibitor is present in an amount to inhibit the digestion of the leptin or leptin functional derivative by one or more pancreatic proteases in the subject. In another embodiment, the above mentioned pancreatic protease inhibitor is aprotinin.

In another embodiment, the above mentioned bile acid or bile acid analog comprises: deoxycholic acid; cholic acid; chenodeoxycholic acid; taurocholic acid; taurochenodeoxycholic acid; glycocholic acid; glycochenocholic acid; 3β-monohydroxychloric acid; lithocholic acid; 3-hydroxy-12-ketocholic acid; 12-3-dihydrocholic acid; ursodesoxycholic acid; or an analog thereof; or any combination thereof. In another embodiment, the above mentioned bile acid or bile acid analog is: deoxycholic acid; taurocholic acid; lithocholic acid; an analog thereof; or any combination thereof. In another embodiment, the above mentioned bile acid or bile acid analog is present in an amount to allow intestinal absorption of the leptin or leptin functional derivative in the subject.

In another embodiment, the above mentioned oral composition further comprises a sweetener. In another embodiment, the above mentioned oral composition further comprises a stimulator of endogenous leptin secretion or a satiety triggering agent. In another embodiment, the above mentioned stimulator of leptin secretion or satiety triggering agent is: glutamine; insulin: secretin; cholecystokinin (CCK); pentagastrin; a glucocorticoid; transretinoic acids; an analog thereof; or any combination thereof.

In another embodiment, the above mentioned stomach acid neutralizing agent is present at a concentration from about 10 mM to about 250 mM.

In another embodiment, the above mentioned bile acid or bile acid analog is present at a concentration from about 1 mg/mL to about 25 mg/mL.

In another embodiment, the above mentioned oral composition is in the form of: a tablet; a pill; a powder; a syrup; a liquid; a food; a dragee; a confectionary; or any combination thereof.

In another embodiment, the above mentioned oral composition is eligible for natural health product status.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows an exemplary standard curve for leptin as measured by enzyme immunoassay;

FIG. 2 shows the effect of oral leptin formulations of the present invention on plasma leptin levels following oral administration in leptin-deficient ob/ob mice;

FIG. 3 shows the effect of administration of different amounts of oral leptin formulations of the present invention on body weight of leptin-deficient ob/ob mice;

FIGS. 4A and 4B shows the effect of administration of oral leptin formulations of the present invention on food intake and body weight, respectively, in leptin-deficient ob/ob mice;

FIG. 5 shows the effect of long-term administration of oral leptin formulations of the present invention on body weight of leptin-deficient ob/ob mice;

FIGS. 6A and 6B show the effect of oral leptin formulations of the present invention on food intake and body weight, respectively, in normal, non-obese wild-type C57BL/6J mice. Arrows indicate time of leptin formulation administration;

FIG. 7 shows plasma leptin levels after various doses of oral administration of leptin in vehicle 2 to wild-type C57BL/6J mice;

FIG. 8 compares the effect of vehicle alone without leptin (triangles) with those of 10 μg of leptin in vehicle 2 (diamonds) and 10 μg of leptin in PBS (squares) on plasma leptin levels in wild-type C57BL/6J mice;

FIG. 9 shows the effect of removal of bicarbonate buffer from an oral combination therapy of the present invention on plasma leptin levels in wild-type C57BL/6J mice;

FIG. 10 shows the effect of removal of bile salt from an oral combination therapy of the present invention on plasma leptin levels in wild-type C57BL/6J mice;

FIG. 11 shows the effect of removal of the anti-protease mix from an oral combination therapy of the present invention on plasma leptin levels in wild-type C57BL/6J mice;

FIG. 12 shows the effect of removal of ethanol from an oral combination therapy of the present invention on plasma leptin levels in wild-type C57BL/6J mice;

FIG. 13 shows the effect of removal of sucrose from an oral combination therapy of the present invention on plasma leptin levels in wild-type C57BL/6J mice;

FIG. 14 shows the effect of different bile acids on plasma leptin levels in wild-type C57BL/6J mice. The effect of taurocholate, cholate and lithocholate is compared with that of deoxycholate in Panels A, B and C respectively;

FIG. 15 shows a comparison of taurocholate present in soluble or micelle form on plasma leptin levels in wild-type C57BL/6J mice;

FIG. 16 shows the effect of a 10-fold reduction in the amount of an anti-protease mix on plasma leptin levels in wild-type C57BL/6J mice;

FIG. 17 shows a comparison between a commercially obtained anti-protease mix and homemade mix of protease inhibitors on plasma leptin levels in wild-type C57BL/6J mice;

FIG. 18 shows the effect of different buffers on plasma leptin levels in wild-type C57BL/6J mice;

FIG. 19 shows the effect of pH of the vehicle on plasma leptin levels in wild-type C57BL/6J mice;

FIG. 20 shows the effect of pH of the vehicle on mouse leptin protection in a simulated gastric environment;

FIG. 21 shows the effect of different anti-proteases on human leptin protection of in a simulated gastric environment;

FIG. 22 shows the effect of different anti-proteases on human leptin protection of in a simulated duodenal environment;

FIG. 23 shows the effect of replacing a commercial anti-protease mix (1 tablet/10 mL) with aprotinin (30 μg and 100 μg) on plasma leptin levels in wild-type C57BL/6J mice measured 30 minutes after oral administration;

FIG. 24 shows the effect of replacing a commercial anti-protease mix with aprotinin on plasma leptin levels in wild-type C57BL/6J mice over 120 days;

FIG. 25 shows the effect of oral leptin on body weight of db/db mice;

FIG. 26 shows the effect of oral leptin on food consumption of db/db mice;

FIG. 27 shows the effect of including glutamine in the oral leptin formulation on plasma leptin levels of wild-type C57BL/6J mice;

FIG. 28 shows the effect of oral rat leptin on the body weight of Male Wistar rats (n=5);

FIG. 29 show the effect of rat leptin administered orally on food intake of Male Wistar rats;

FIG. 30 shows the effect of rat leptin administered orally on plasma leptin levels in rats;

FIG. 31 shows the effect of rat or human leptin administered orally on the body weight and food intake of rats;

FIG. 32 shows the effect of mouse leptin administered orally with food on plasma leptin levels of Wistar rats;

FIG. 33 shows the effect of human leptin administered orally with food on plasma leptin levels of Wistar rats;

FIG. 34 shows the endogenous plasma leptin levels of rats after ingestion of standard food (i.e., Purina Chow™) devoid of leptin (but soaked in vehicle 3);

FIG. 35 shows the plasma leptin levels of rats after ingestion of standard food (i.e., Purina Chow™) soaked in rat leptin (150 μg) compared with oral administration of rat leptin (150 μg) without food (leptin without food, diamonds; leptin with food, squares);

FIG. 36 shows a comparison between oral and intraperitoneal (IP) administration of leptin. Diamonds correspond to weight variation over the three days after IP saline injection, squares correspond to weight variation over the three days after IP mouse leptin (2.5 μg) injection; triangles correspond to weight variation over the three days after oral vehicle force feeding; and circles correspond to weight variation over the three days after oral mouse leptin (2.5 μg) force feeding;

FIG. 37 shows a comparison between oral and intraperitoneal (IP) administration of leptin. It presents the average daily body weight changes in the mice of FIG. 36 over three days;

FIG. 38 shows a comparison between oral and intraperitoneal (IP) administration of leptin. It presents food consumption per day of the mice of FIGS. 36-37;

FIG. 39 shows body weight variations of C57BL/6J mice receiving no treatment (Panel A, Control mice; n=2), daily oral administration of vehicle alone (without leptin) (Panel B; n=3), or vehicle containing leptin (1 μg) over one month (Panel C; n=4);

FIG. 40 shows images taken of stomach tissue via light microscopy of the gastric wall in Controls, Vehicle-treated and Leptin-treated C57BL/6J mice (panels “C”, “V”, and “L”, respectively). “Lu” represents the gastric lumen;

FIG. 41 shows images taken of stomach tissue via electron microscopy of the gastric mucosa of a leptin-treated C57BL/6J mouse (“L”, referring to both upper and lower panels). “Lu” represents gastric lumen; “N” represents nucleus; “bv” represents blood vessels; “sg” represents secretory granules; “j” represents intercellular junctions;

FIG. 42 shows images taken of duodenum tissue via light microscopy in Controls, Vehicle-treated and Leptin-treated C57BL/6J mice (panels “C”, “V”, and “L”, respectively). “Lu” represents the gastric lumen.

FIG. 43 shows images taken of duodenum tissue via electron microscopy of the duodenal mucosa of a leptin-treated C57BL/6J mouse (“L”, referring to both upper and lower panels). “Mv” represents microvilli; “j” represents intercellular junctions; “Lu” represents duodenal lumen.

FIG. 44 shows images taken of liver tissue via light microscopy in Controls, Vehicle-treated and Leptin-treated C57BL/6J mice (panels “C”, “V”, and “L”, respectively).

FIG. 45 shows images taken of liver tissue via electron microscopy from a leptin-treated C57BL/6J mouse (“L”, referring to both upper and lower panels). “N” represents nucleus; “m” represents mitochondria; “bc” represents bile canaliculi; “RER” represents rough endoplasmic reticulum.

FIG. 46 shows the effect of oral leptin in vehicle 3 on body weight stabilization in ob/ob mice over one month. Panels A and B show experiments performed in December 2010 and in March 2011, respectively; with triangles, diamonds and squares corresponding to individual animals each receiving the same treatment;

FIG. 47 shows the effect of oral leptin in vehicle 3 on mean body weight stabilization in ob/ob mice over 16 days; and

FIG. 48 shows A) an alignment of leptin fragments of Annex 2; B) an alignment of processed (i.e. without signal peptide) human (SEQ ID NO: 3), mouse (SEQ ID NO: 5) and rat (SEQ ID NO: 114) leptin. A consensus sequence derived from this alignment is also presented (SEQ ID NO: 115), wherein X can be any amino acid; and C) an alignment of human leptin sequences presenting polymorphisms (SEQ ID NOs: 56-61). A consensus sequence derived from this alignment is also presented (SEQ ID NO: 116), wherein X can be any amino acid.

### DESCRIPTION OF ILLUSTRATIVE EMBODIMENTS

### Definitions

In the present description, a number of terms are extensively utilized. In order to provide a clear and consistent understanding of the specification and claims, including the scope to be given such terms, the following definitions are provided.

Unless defined otherwise, the scientific and technological terms and nomenclature used herein have the same meaning as commonly understood by a person of ordinary skill to which this invention pertains. Commonly understood definitions of molecular biology terms can be found, for example, in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), and Alberts et al., Molecular Biology of the Cell, 4th edition, Garland science, New-York, 2002. Generally, the methods traditionally used in molecular biology are common methods used in the art and can be found in reference manuals such as Sambrook et al. (2000, Molecular Cloning—A Laboratory Manual, Third Edition, Cold Spring Harbour Laboratories); and Ausubel et al. (1994, Current Protocols in Molecular Biology, John Wiley. Sons, New-York); and in the journal Cold Spring Harbor Protocols.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

Leptin

As used herein, the term “leptin” refers to the secreted form of the native leptin polypeptide/protein sequence (e.g., human leptin sequence and orthologs thereof (e.g., Table I, Annexes 1 and 3)). The present invention also encompasses functional derivatives of leptin which include variants (e.g., functional fragments/variants (e.g., Annex 2)), analogs and prodrugs thereof.

TABLE I
GenBank accession numbers for human leptin

| | |
|---|---|
| Genomic | NG_007450.1 RefSeqGene (Range 5001 . . . 21352) |
| sequence | |
| mRNA | NM_000230.2 → NP_000221.1 leptin precursor |
| and protein | Source sequence(s) |
| sequences | AC018635, BU752306, DA762132, U43653 |
| | Consensus CDS: CCDS5800.1 |
| | UniProtKB/TrEMBL: A4D0Y8 |
| | UniProtKB/Swiss-Prot: P41159 |
| | Related Ensembl: ENSP00000312652, ENST00000308868 |

As used herein, “protein” or “polypeptide” means any peptide-linked chain of amino acids, regardless of post-translational modifications (e.g., acetylation, phosphorylation, glycosylation, sulfatation, sumoylation, prenylation, ubiquitination, etc). When referring to nucleic acid molecules, proteins or polypeptides, the term “native” refers to a naturally occurring nucleic acid or polypeptide. A homolog or ortholog is a gene sequence encoding a polypeptide isolated from an organism other than a human being. Similarly, a homolog of a native polypeptide is an expression product of a gene homolog. The amino acid sequence of the human leptin protein (i.e., the processed protein having residues 22-167 of the human leptin precursor protein) was used as the basis of a Blast protein search (GenBank CDS translations+PDB+SwissProt+PIR+PRF) and the sequences of the top 100 queries are shown in Annex 3.

A “leptin protein” or “leptin polypeptide” is an expression product of a leptin nucleic acid (e.g., ob gene) such as a native human leptin protein, a natural splice variant of a leptin gene, an allelic variant of a leptin gene, a leptin molecule that has been processed (e.g., to remove a signal sequence) or a leptin protein homolog or ortholog (e.g., a mouse leptin protein) that shares at least 60% (but preferably, at least 65, 70, 75, 80, 85, 86, 87, 88, 89, 90, 91, 92, 93, 94, 95, 96, 97, 98, 99, 100%) amino acid sequence identity with a leptin protein and displays functional activity of a native leptin protein. For the sake of brevity, the units (e.g., 66, 67 . . . 81, 82% . . . ) have not been specifically recited but are nevertheless considered within the scope of the present invention.

Leptin Functional Derivatives

As indicated above, the present invention also encompasses functional derivatives of leptin. As used herein in the context of leptin, a “leptin functional derivative” refers to a molecule that retains (either in its present form or via an in vivo processing step) the ability to bind to an intestinal leptin receptor and maintain a biological activity (either functional or structural) that is substantially similar to that of native leptin. Functional derivatives of leptin may be obtained naturally or synthetically and include variants (e.g., functional fragments), analogs and prodrugs thereof.

As used herein, the term “variant” when used in the context of leptin or in the expression “leptin variant” or “variant of leptin” refers to any peptide, polypeptide or protein with a sequence that is partially identical to that of a native leptin protein or polypeptide, and retaining a biological activity of the leptin protein or polypeptide that is substantially similar to that of the original sequence. Such variants include polypeptides having amino acid substitutions, deletions, truncations or additions of one or more amino acids as well as posttranslational modifications (e.g., acetylation, phosphorylation, glycosylation, sulfatation, sumoylation, prenylation, ubiquitination, etc), provided that a biological activity of the leptin protein is conserved. Where applicable, the substituting amino acid generally has chemico-physical properties, which are similar to that of the substituted amino acid. The similar chemico-physical properties include similarities in charge, bulkiness, hydrophobicity, hydrophilicity and the like. As used herein the term “functional fragment” denotes, in the context of a fragment of leptin, a specific type of leptin variant, namely a molecule that retains a biological activity that is substantially similar to that of the original sequence (e.g., native leptin) but that lacks at least a part of this original sequence. This fragment may be a natural fragment (e.g., a naturally occurring isoform, allelic variant or splice variant) or may be prepared synthetically (e.g., in vitro). Functional leptin fragments are described previously (Malendowicz et al., 2003; Malendowicz et al., 2004a; Malendowicz et al., 2004b; Hanew, 2003; Oliveira et al., 2005; Markowska et al., 2004 and Markowska et al., 2005). The mouse leptin of SEQ ID NO: 1, the human leptin of SEQ ID NO: 113 and the rat leptin of SEQ ID NO: 114 used in Examples below are examples of leptin variants encompassed by the present invention. The leptin consensus sequence of SEQ ID NO: 115 is also such a variant. In this variant, the Xs can be any amino acids. In a more specific embodiments, X1 can be Q or H, X2 can be A or S, X3 can be K or R, X4 can be R or K, X5 can be S or T, X6 can be V or I, X7 can be L or M; X8 can be Q or R, X9 can be L or I, X10 can be A or S, X11 can be N or H, X12 can be L or V, X13 can be S or H, X14 can be Q or W, X15 can be T or A, X16 can be S or R, X17 can be Q or E, X18 can be K or T, X19 can be P or L, X20 can be E or D, X21 can be D or G, X22 can be L or G, X23 can be I or M, X24 can be Q or W, X25 can be V or L; and X26 can be E or G. The processed version (i.e., without signal peptide) of the leptin consensus sequence of SEQ ID NO: 116 is also such a variant. In this variant, the Xs can be any amino acids. In a more specific embodiment, X1 can be Q or absent; X2 can be D or N, X3 can be Q or R, and X4 can be W or E.

Amino acid sequence variants of leptin can be prepared by mutations in the DNA encoding same. Such variants include, for example, deletions from, or insertions/substitutions of, residues within the amino acid sequence of leptin. Any combination of deletions, insertions, and substitutions can also be made to arrive at the final construct, provided that the final construct possesses the desired activity. Techniques for making substitution mutations at predetermined sites in DNA having a known sequence are well known in the art and include, for example, site-specific mutagenesis. Site-specific mutagenesis allows the production of leptin variants through the use of specific oligonucleotide sequences that encode the DNA sequence of the desired mutation. In general, the technique of site-specific mutagenesis is well known in the art, as exemplified by publications such as Adelman et al., DNA 2:183 (1983) and Ausubel et al., “Current Protocols in Molecular Biology”, J. Wiley & Sons, NY, NY, 1996.

Amino acid sequence deletions generally range from about 1 to 30 residues, more preferably 1 to 10 residues, and typically are contiguous. Amino acid sequence insertions include amino and/or carboxyl-terminal fusions of from one residue to polypeptides of essentially unrestricted length, as well as intra-sequence insertions of single or multiple amino acid residues. Intra-sequence insertions can range generally from about 1 to 10 residues, more preferably 1 to 5. Amino acid substitutions are those in which at least one amino acid residue in a polypeptide (e.g., leptin) has been removed and a different residue inserted in its place. Such substitutions preferably are made in accordance with the following Table II, when it is desired to modulate finely the characteristics of the polypeptide.

TABLE II
Exemplary amino acid substitutions

| Original | Exemplary |
|---|---|
| Residue | Substitutions |
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in functional or immunological identity can be made by selecting substitutions that are less conservative than those in Table II, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that, in general, are expected to provide substantial changes in functional or immunological identity are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine.

Some deletions, insertions, and substitutions are not expected to produce radical changes in the characteristics of the polypeptides of the present invention. However, when it is difficult to predict the exact effect of the substitution, deletion, or insertion in advance of doing so, one skilled in the art will appreciate that the effect will be evaluated by routine screening assays. For example, a variant typically is made by site-specific mutagenesis of a native leptin encoding-nucleic acid, expression of the variant nucleic acid in recombinant cell culture, and, optionally, purification from the cell culture, for example, by immunoaffinity adsorption on a column (to absorb the variant by binding it to at least one remaining immune epitope). The activity of the cell lysate or purified leptin molecule variant is then screened in a suitable screening assay for the desired characteristic. For example, a change in the immunological character of the polypeptide molecule, such as affinity for a given antibody, is measured by a competitive type immunoassay. Modifications of such protein properties as stability, solubility, hydrophobicity, binding affinity, susceptibility to proteolytic degradation or the tendency to aggregate are assayed by methods known to the skilled person.

Herein, the terms “analog” and “chemical analog” are used interchangeably and when used in association with a component of the oral combination therapies of the present invention (e.g., leptin analog, sodium bicarbonate analog, bile acid analog, deoxycholate analog, pancreatic protease analog, aprotinin analog, ethanol analog, aspartame analog, sucralose analog, stevia rebaudiana extract analog, sucrose analog, glucose analog, fructose analog, sugar cane analog, high fructose corn syrup (HFCS) analog, agave syrup analog, honey analog and maple syrup analog) is meant to cover the specific component as chemically modified (e.g., by additional chemical moieties not normally part of the specific component). Such moieties could affect the physico-chemical characteristic of the analog (i.e., solubility, absorption, half life and the like, decrease of toxicity). Such moieties are exemplified in “Remington: The Science and Practice of Pharmacy” by Alfonso R. Gennaro, 2003, 21st edition, Mack Publishing Company. Methods of coupling these chemical physical moieties to a polypeptide are well known in the art. For instance, and not without being so limited, the terms “analog” and “chemical analog” include any inorganic or organic salts of this component that may be suitable for the oral combination therapies of the present invention.

Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium, ammonium and the like, as well as from nontoxic organic amines, such as N,N′-dibenzylethylenediamine, N-methylglucamine, chloroprocaine, choline, diethanolamine, ethylenediamine, procaine and the like. For example, the recitation “bicarbonate” or bicarbonate buffer” of the present invention may be used as sodium, potassium, magnesium, calcium or ammonium salt. In another embodiment, the bile acids of the present invention (e.g., deoxycholate) may be used as sodium or sodium monohydrate salts, or other suitable salts.

More specifically, and without being so limited, a leptin polypeptide or protein that contains a total or partial sequence of leptin with the addition of other groups such as amino acids, amides, lipids and carbohydrates, which are not normally found (e.g., in vivo) in native leptin, are considered analogs of leptin.

As used herein, a “prodrug” in the context of a leptin prodrug refers to a leptin-related molecule administered in an inactive (or significantly less active) form, which is converted into an active or more active form of leptin in vivo following oral administration.

As used herein, the terms “molecule”, “compound”, “agent” or “ligand” are used interchangeably and broadly to refer to natural, synthetic or semi-synthetic molecules or compounds. The term “compound” therefore denotes, for example, chemicals, macromolecules, cell or tissue extracts (from plants or animals) and the like. Non-limiting examples of compounds include peptides, antibodies, carbohydrates, nucleic acid molecules and pharmaceutical agents. The compound can be selected and screened by a variety of means including random screening, rational selection and by rational design using, for example, protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”.

The term “subject” or “patient” as used herein refers to an animal, preferably a mammal, and most preferably a human who is the object of treatment, observation or experiment. As used herein, “mammal” includes humans and both domestic animals such as laboratory animals and household pets, (e.g., cats, dogs, swine, cattle, sheep, goats, horses, rabbits), and non-domestic animals such as wildlife and the like.

As used herein, the term “purified” refers to a molecule (e.g., a leptin polypeptide or functional fragment thereof) having been separated from a component of the composition in which it was originally present. The term purified can sometimes be used interchangeably with the term “isolated”. Thus, for example, a “purified or isolated polypeptide or polynucleotide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. Therefore, the terms “separating”, “purifying” or “isolating” refers to methods by which one or more components of the biological sample are removed from one or more other components of the sample. Sample components include nucleic acids in a generally aqueous solution that may include other components, such as proteins, carbohydrates, or lipids. A separating or purifying step preferably removes at least about 70% (e.g., 70, 75, 80, 85, 90, 95, 96, 97, 98, 99, 100%), more preferably at least about 90% (e.g., 90, 91, 92, 93, 94, 95, 96, 97, 98, 99, 100%) and, even more preferably, at least about 95% (e.g., 95, 96, 97, 98, 99, 100%) of the other components present in the sample from the desired component. For the sake of brevity, the units (e.g., 66, 67 . . . 81, 82, . . . 91, 92% . . . ) have not systematically been recited but are considered, nevertheless, within the scope of the present invention.

As used herein, the term “pharmaceutically acceptable” (e.g., in pharmaceutically acceptable carrier) refers to molecular entities and compositions that are physiologically tolerable and do not typically produce an allergic or similar unwanted reaction, such as gastric upset, instability, irritation, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans. The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin. As used herein, “medically acceptable” refers to ingredients suitable for use by oral administration (e.g., in contact with mouth, esophagus, stomach, intestines) without undue toxicity, incompatibility, instability, irritation, allergic response, or the like.

As used herein, the expressions “diseases, conditions or phenotypes that are associated with or that can be ameliorated by leptin” or “diseases, conditions or phenotypes that are associated with low plasma leptin levels or that can be ameliorated by increasing plasma leptin levels” refer to diseases, conditions, phenotypes, syndromes or disorders that are associated with either low plasma leptin levels (e.g., hypoleptinemic state associated with an abnormality in the endogenous leptin pathway) or would benefit from the administration of oral leptin formulations of the present invention. As used herein, the terms “condition”, “syndromes”, “disease” and “disorder” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. Examples of diseases and/or conditions that are associated with or that can be prevented, treated or managed by leptin include weight gain, obesity, type 1 and/or type 2 diabetes, depression, leptin-deficient state, hypothalamic amenorrhea, cardiovascular disease, any hypoleptinemic disease, or any cases in which the subjects are required to lose body weight or increase leptinemia (plasma leptin levels) in order to improve health.

The oral compositions of the present invention can also prevent, treat or manage one or more symptoms/phenotypes of the foregoing diseases and/or conditions. For instance, and without being so limited, they are useful for lowering blood glucose levels observed in diabetes type 1, in a way independent from insulin; to lower blood glucose levels observed in diabetes type 2, by decreasing body weight and improving glycemic control; improve lipid profile in patients with cardiac complications; to restore fertility including spermatogenesis and ovulation in patients suffering from infertility resulting from low leptin plasma levels; to increase plasma leptin levels in subjects in need thereof; to improve depressive states in patients suffering from psychological troubles resulting from or aggravated by leptin deficiency; to lowering appetite in obese or normal patients; to controlling, losing or maintaining body weight; to decrease and/or control the rate of weight gain in a subject; or to control or increase the rate of energy expenditure in a subject.

Oral leptin formulations of the present invention are also useful for controlling, losing or maintaining body weight. As used herein, “losing or maintaining” is defined as, but not limited to, decreasing or keeping stable the body weight to either keep or improve general health for aesthetic or medical purposes. The skilled person would understand that weight loss of subjects receiving the oral leptin formulations of the present invention can depend on parameters such as the age, gender, diet, existing medical condition(s), the duration and nature of the treatment.

As used herein, a “stable weight” means an amount of leptin in his vehicle that is sufficient to maintain a stable body weight following a weight loss. This amount will vary with the patient being treated, the age, gender or other medical condition existing, the duration and nature of the treatment, and like factors.

As used herein, the terms “treat”, “treating” and “treatment” contemplate an action that occurs while a patient is suffering from the specified disease and/or condition, which reduces the severity of the disease or disorder or of one or more symptom/phenotype thereof, or retards or slows the progression of the disease and/or condition or of one or more symptom/phenotype thereof.

As used herein, the terms “prevent”, “preventing” and “prevention” contemplate an action that occurs before a patient begins to suffer from the specified disease or disorder, which delays the appearance of the disease and/or condition or of one or more symptom/phenotype thereof, or inhibits (completely or partially) or reduces the severity of the disease and/or condition or of one or more symptom/phenotype thereof.

As used herein, the terms “manage”, “managing” and “management” encompass preventing the recurrence of the specified disease and/or condition or of one or more symptom/phenotype thereof in a patient who has already suffered from the disease and/or condition, and/or lengthening the time that a patient who has suffered from the disease or disorder remains in remission. The terms encompass modulating the threshold, development and/or duration of the disease and/or condition or of one or more symptom/phenotype thereof, or changing the way that a patient responds to the disease and/or condition or of one or more symptom/phenotype thereof.

The term “therapeutically effective amount” of a compound is an amount sufficient to provide a therapeutic benefit in the treatment or management of a disease and/or condition or of one or more symptom/phenotype thereof. The term “therapeutically effective amount” can encompass an amount that that directly treats or manages the disease and/or condition or one or more symptom/phenotype thereof, or enhances the therapeutic efficacy of another therapeutic agent.

As used herein, “prophylactically effective amount” of a compound is an amount sufficient to prevent a disease and/or condition, or one or more symptoms associated with the disease and/or condition, or prevent its recurrence. The phrase “prophylactically effective amount” an encompass an amount that improves overall prophylaxis or enhances the prophylactic efficacy of another prophylactic agent.

Compositions and oral combination therapies of the present invention may be in the form of liquid solutions or suspension(s), tablets or capsules, dragees, or powders, an may include an inert diluent or an edible carrier. In one embodiment, the active compounds of the present invention can be incorporated with excipients and used in the form of tablets, troches, or capsules. In an embodiment, pharmaceutically compatible binding agents, and/or adjuvant materials can be included as part of the oral combination therapy or compositions.

It may be advantageous to formulate the compositions or oral combination therapies of the present invention in one or more dosage unit form(s) for ease of administration and uniformity of dosage(s). “Dosage unit form” as used herein refers to physically discrete units suited as unitary dosages for the subject to be treated; each unit containing a predetermined quantity of active compound(s) calculated to produce the desired therapeutic effect. Data obtained from cell culture assays and animal studies can be used in formulating a range of dosage(s) for use in humans. Toxicity and therapeutic efficacy can be determined by measuring the LD50 (the dose lethal to 50% of the population) and the ED50 (the dose therapeutically effective in 50% of the population). The dosage of such compounds lies preferably within a range of circulating concentrations that include the ED50 with little or no toxicity. The dosage may vary within this range depending upon the dosage form employed.

Unless otherwise indicated, a percentage refers to a percentage by weight for solid (i.e., % (W/W)) or by volume for liquid (i.e., % (V/V)).

A “functional food” is similar in appearance to, or may be, a conventional food that is consumed as part of a usual diet, and is demonstrated to have physiological benefits and/or reduce the risk of disease and/or condition or of one or more symptom/phenotype thereof beyond basic nutritional functions, i.e. they contain a bioactive compound. As used herein, “beverages” include powers, syrups and concentrated for the production thereof.

Combination Therapy for Oral Leptin Administration

The present invention relates to oral combination therapies for the delivery of orally administered exogenous leptin to the bloodstream of a subject in its active form. “Active form” as used herein means that the biological activity of the exogenous leptin compounds that are orally administered are substantially retained upon delivery to the bloodstream of the subject.

In one aspect, the oral combination therapies of the present invention comprise: (a) leptin or a leptin functional derivative; (b) a stomach acid neutralizing agent (e.g., a buffer) for protecting the leptin or leptin functional derivative from the gastric pepsin; (c) a pancreatic protease inhibitor for protecting the leptin or leptin functional derivative from pancreatic proteolytic enzymes (e.g., trypsin, chymotrypsin, carboxypeptidase, elastase); and (d) a bile acid or a bile acid analog for facilitating the intestinal absorption of the leptin or leptin functional derivative.

“Oral combination therapy” (or “oral co-therapy”) as used herein refers to one or more compounds or agents (e.g., (a) leptin or a leptin functional derivative; (b) a stomach acid neutralizing agent; (c) a pancreatic protease inhibitor; and (d) a bile acid or a bile acid analog) which are to be administered orally to a subject either simultaneously or sequentially within a relatively short time period, so that the one or more compounds can be present together within the gastrointestinal tract of the subject. Substantially simultaneous administration can be accomplished, for example, by administering to the subject a single composition having a fixed ratio of each (a)-(d) or in multiple, single compositions of (a)-(d).

In one embodiment, the oral combination therapy can comprise the one or more compounds (e.g., compounds (a)-(d)) in separate containers. In another embodiment, the oral combination therapy can comprise the one or more compounds (e.g., compounds (a)-(d)) formulated together as a single oral composition. In other embodiments, two or more of the compounds (e.g., compounds (a)-(d)) can be combined in a single container with the remaining compounds packaged separately. For example, (a) and (c) can be present in one container while (b) and (d) can be present in a single container or in separate containers. The person of ordinary skill in the art would be able to adapt the number/content of containers of the oral combination therapies of the present invention in order to suit particular needs (e.g., maximize convenience and/or shelf-life; minimize production cost).

In one embodiment, the oral combination therapies of the present invention can be combined with an agent which stimulates, promotes, or enhances endogenous leptin secretion in the subject being administered. In another embodiment, the oral combination therapies of the present invention can comprise an agent such as a sweetener for promoting, enhancing, or improving adherence of a subject to treatment.

Leptin and Leptin Functional Derivatives

The oral combination therapies of the present invention comprise leptin or a leptin functional derivative (i.e., a variant (e.g., functional fragment), analog or prodrug thereof).

In one embodiment, the oral combination therapies of the present invention can comprise a native leptin polypeptide, such as human leptin and orthologs thereof (e.g., Table I, Annexes 1 and 3). In another embodiment, the oral leptin compositions of the present invention can comprise recombinant leptin. In another embodiment, the oral leptin compositions of the present invention can comprise the precursor and/or processed leptin (e.g., those described in Annex 1).

In another embodiment, the leptin or leptin functional derivatives of the present invention can include molecules such as: (i) leptin or leptin functional derivatives bound or coupled to a protective chaperone; (ii) variants/fragments of leptin (e.g., human leptin); (iii) leptin analogs; (iv) other variations of leptin not mentioned here; as long as the molecules retain their ability to bind to the intestinal leptin receptor can be delivered to the bloodstream of a subject their active forms. The chaperone polypeptide can be a polypeptide capable of binding to or interacting with leptin or leptin functional derivative (e.g., a leptin receptor or functional fragment thereof).

In another embodiment, the oral combination therapies or compositions of the present invention comprise leptin bound or coupled (e.g., covalently or non-covalently) to a protective chaperone such as a polypeptide, as long as the binding or coupling does not interfere with the interaction of the leptin with its duodenal leptin receptor and its subsequent internalization. For example, a chaperone polypeptide can be a polypeptide capable of binding to or interacting with leptin (e.g., a leptin receptor or functional fragment thereof). In another embodiment, the oral leptin combination therapies or compositions of the present invention comprise leptin covalently bound to the leptin binding domain (LBD) of the human leptin receptor, optionally with a linker segment (e.g., a flexible glycine-serine linker as described in Carpenter et al., 2009). In another embodiment, the chaperone polypeptide can be an Fc fragment from an immunoglobulin gamma chain attached to the N-terminal portion of leptin (e.g., the “engineered leptin immunofusins” described in Lo et al., 2005).

In another embodiment, the oral combination therapies or compositions of the present invention can comprise variants/fragments of leptin (e.g., human leptin) having a biological activity of native leptin (e.g., those described in Annex 2), as long as the variants/fragments can be absorbed by intestinal cells and retain biological activity. In another embodiment, the oral leptin formulations of the present invention comprise synthetic fragments/variants of leptin, such as the leptin-like synthetic peptide amide, [D-Leu-4]-OB3, which corresponds to residues 116-122 of leptin with a substitution of the Leu at position 4 with its D-isomer (Grasso et al., 2001). This region corresponds to the amino acid sequence SCHLPWA in human leptin and SCSLPQT of mouse leptin. In another embodiment, the oral leptin formulations of the present invention comprise the fragments of leptin (e.g., human leptin) disclosed in U.S. Pat. Nos. 6,777,388; 7,186,694 and 7,208,572. These fragments include the peptides defined by residues 21-35, 31-45, 41-55 and 51-65, 61-75, 71-85, 81-95, 91-105, 106-120, 116-121, 116-130, 126-140, 136-150, 146-160, and 156-167 of native leptin (e.g., human leptin). Other leptin polypeptides have been identified such as those disclosed by Basinski et al., in PCT applications WO 96/23515 and WO 96/23517.

In another embodiment, the oral combination therapies or compositions of the present invention can comprise leptin variants or analogs that can antagonize or decrease/interfere with the activity of the endogenous leptin receptor. For example, these leptin receptor antagonists (e.g., competitive antagonists) may bind to a leptin receptor with an affinity similar to that of wild-type or native leptin, and yet be devoid of biological activity. Examples of antagonistic leptin variants include leptin polypeptides having one or more alanine substitution mutation(s) at residues 39-41 or 39-42 of native leptin, as described in Solomon et al., 2006. Such antagonistic leptin variants could be used as anti-cancer/anti-tumoral agents. In another embodiment, the oral combination therapies of the present invention comprise antagonistic leptin variants and are for preventing, treating or managing cancer or tumor growth.

Modifying the amino acid sequence of leptin (e.g., amino acid insertions, substitutions, deletion and/or truncations) to achieve a desired property would be within the capacities of the skilled person and such modified leptin variants are considered within the scope of the present invention. In one embodiment, modifying one or more amino acids of the leptin portion that binds to its receptor may increase or decrease the binding capacity of leptin to its receptor. In another embodiment, modifying amino acids outside the binding portion, i.e., the amino-acids that stabilize the structure of the whole protein, may increase or decrease leptin half-life.

Stomach Acid-Neutralizing Agent

In another aspect, the oral combination therapies of the present invention comprise a stomach acid-neutralizing agent, such as one or more chemical agent(s) capable of decreasing acidity or raising the pH in the gastric juice by neutralizing stomach acid (e.g., hydrochloric acid) present therein. Without being bound by theory, increasing the pH of the gastric juice can inhibit the proteolytic activity of proteolytic enzymes present in the gastric cavity that may otherwise degrade the orally administered leptin or leptin functional derivative in the stomach. The major proteolytic enzyme in the stomach is pepsin, which is a member of the aspartate protease family and whose precursor form (pepsinogen) is released by chief cells in the stomach. While pepsin functions optimally at about pH 2, raising the pH of the gastric juice above pH 5 is known to inactivate the enzyme, and raising the pH above pH 7 is known to denature the enzyme. In one embodiment, the stomach acid-neutralizing agent can raise the pH of the gastric juice of the subject being administered an oral combination therapy of the present invention by about 1 pH unit; by about 2 pH units; or by about 3 or more pH units.

In another embodiment, the stomach acid-neutralizing agent can be a buffer with a buffering capacity to increase the pH of the gastric juice to a level sufficient to inactivate gastric pepsin. In another embodiment, the buffer comprises a mixture of a weak acid and its conjugate base, or a weak base and its conjugate acid. In another embodiment, the buffer can comprise a phosphate buffer (e.g., NaPO₄); a bicarbonate buffer (e.g., NaHCO₃); a citrate buffer; an acetate buffer (CH₃COOH); or any combination thereof.

In another embodiment, the stomach acid-neutralizing agent can comprise an amphoteric and amphiprotic compound such as sodium bicarbonate (NaHCO₃). Sodium bicarbonate can neutralize acid when in an acidic environment to become H₂CO₃ and can also neutralize bases when the pH is superior to 8.3 to become CO₃²⁻. H₂CO₃ resulting from acid neutralization may also prevent hyper-alkalization of the digestive tract.

In another embodiment, the stomach acid-neutralizing agent can comprise a weak acid and/or a weak base such as KH₂PO₄ and/or K₂HPO₄. In another embodiment, the stomach acid-neutralizing agent can be a commercially available antacid. In another embodiment, the stomach acid-neutralizing agent can further comprise a pepsin inhibitor such as pepstatin and/or 1,1-bis(diazoacetyl)-2-penylethane.

In another embodiment, the stomach acid-neutralizing agent is present in an amount able to inhibit the digestion of the leptin or leptin functional derivative of the present invention by the endogenous gastric pepsin of said subject. In another embodiment, the amount of the stomach acid-neutralizing agent (e.g., sodium bicarbonate or sodium phosphate) in the oral combination therapies of the present invention can vary from about 10 mM to about 250 mM; about 10 mM to about 125 mM; or about 50 mM to about 120 mM; or about 50 mM to about 115 mM; or about 50 mM to about 110 mM; or about 50 mM to about 105 mM; or about 50 mM to about 100 mM; or about 50 mM to about 95 mM. In another embodiment, the amount of stomach acid-neutralizing agent in the oral combination therapies of the present invention can vary from any one of about 10, 15, 20, 25, 30, 35, 40, 45, 50, 55, 60, 65, 70 and 75 mM, to any one of about 80, 85, 90, 95, 100, 105, 110, 115, 120 and 125 mM. In another embodiment, the concentration of the stomach acid-neutralizing agent (e.g., sodium bicarbonate or sodium phosphate) in the oral leptin formulation is about 125 mM. In another embodiment, the concentration of the stomach acid-neutralizing agent (e.g., sodium bicarbonate or sodium phosphate) in the oral leptin formulation is about 100 mM. Other concentration ranges falling within 10 mM and 125 mM, which are not specifically recited here for brevity, are nevertheless included within the present invention.

Pancreatic Protease Inhibitors

In another aspect, the compositions of the present invention comprise an amount of a pancreatic protease inhibitor (protective agent) capable of inhibiting the activity of proteolytic enzymes (e.g., secreted by the gastric mucosa and/or the pancreas) into the digestive tract. Without being bound by theory, inhibiting these enzymes (e.g., pancreatic proteolytic enzymes) protects the orally administered leptin or leptin functional derivative from degradation at the level of the duodenum. A amount of a pancreatic protease inhibitor of the present invention capable of inhibiting the activity of proteolytic enzymes into the digestive tract is an amount which can prevent or reduce the activity of proteolytic enzymes present in the digestive tract to such an extent as to protect orally administered leptin or leptin functional derivative from degradation so that it can be delivered to the blood stream in an active form. In one embodiment, the protective agent includes a pancreatic protease inhibitor or a combination of pancreatic protease inhibitors. In another embodiment, the protective agent includes irreversible and/or reversible protease inhibitors. In another embodiment, the pancreatic protease inhibitors can be a competitive protease inhibitor; a non-competitive protease inhibitor; a peptide; a polypeptide; a protein; or any combination thereof.

In another embodiment, the pancreatic protease inhibitor of the present invention can be comprised in a mixture or cocktail of protease inhibitors which can inhibit a broad spectrum of proteases, including aspartate, serine and/or cysteine proteases. For example, the mixture or cocktail of protease inhibitors of the present invention can include a mixture of protease inhibitors selected from aprotinin, bestatin, calpain inhibitor I and/or II, chymostatin, E-64 (N—[N-(L-3-Trans-carboxirane-2-carbonyl)-L-leucyl]-agmatine), leupeptin (N-acetyl-L-leucyl-L-leucyl-L-argininal), α2-macroglobulin, Pefabloc™ SC (4-(2-Aminoethyl)-benzenesulfonyl fluoreide, hydrochloride), pepstatin, PMSF (phenylmethanesulfonylfluoride or phenylmethylsulfonyl fluoride), TLCK-HCl (tosyllysine chloromethyl ketone—hyfrochloride), trypsin inhibitor (from chicken, egg white) and trypsin inhibitor (from soybean), or any combination thereof. In another embodiment, the above-mentioned protease inhibitors may be present in a pre-mixed liquid or tablet form (e.g., Complete™ Mini, Roche Diagnostics).

In another embodiment, the oral combination therapies of the present invention can comprise a mixture or cocktail of protease inhibitors such as aprotinin (e.g., 10 μg/mL); alpha2-macroglobulin (e.g., 1 μg/mL); leupeptin (e.g., 10 μg/mL); chymostatin (10 μg/mL); trypsin inhibitor (10 μg/mL); and PMSF (20 μg/mL).

In another embodiment, the oral combination therapies of the present invention can comprise at least one pancreatic protease inhibitor such as a trypsin inhibitor; a chymotrypsin inhibitor; a carboxypeptidase inhibitor; an elastase inhibitor; or any combination thereof. In another embodiment, the pancreatic protease inhibitor is present in an amount to sufficiently inhibit the digestion of the leptin or leptin functional derivative by one or more pancreatic proteases in the subject being administered the oral combination therapy. In another embodiment, the pancreatic protease inhibitor is a trypsin inhibitor such as aprotinin.

Bile Acid or Bile Acid Analog

In another aspect, the present oral combination therapies of the present invention can comprise one or more agents capable of enhancing leptin or leptin functional derivative absorption by the digestive tract. In one embodiment, such an agent is a chemical agent capable of increasing leptin uptake by epithelial cells of the intestinal mucosa. In another embodiment, the agent capable of increasing leptin uptake by epithelial cells of the intestinal mucosa is a bile acid or bile acid analog.

“Bile acid” as used herein includes steroid acids, and salts thereof, found in the bile of an animal (e.g., a human), including, for example, cholic acid, lithocholic acid, lithocholate, cholate, deoxycholic acid, deoxycholate, hyodeoxycholic acid, hyodeoxycholate, glycocholic acid, glycocholate, taurocholic acid, taurocholate and the like. Taurocholic acid and/or taurocholate are referred to herein as TCA. Unless otherwise indicated, the terms “bile acid”, “bile salt”, “bile acid/salt”, “bile acids”, “bile salts”, and “bile acids/salts” are used interchangeably herein. For example, any reference to a bile acid used herein includes reference to a bile acid or a salt thereof. Furthermore, it is to be understood that as used herein, “bile acids” include bile acids conjugated to an amino acid (e.g., glycine or taurine). For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Also included in the term “bile acid” are different physical forms or arrangements of the bile acid (e.g., soluble, lyophilized, micelle).

“Bile acid analog” as used herein refers to a bile acid which can be used as part of the oral combination therapy of the present invention which has been chemically modified (e.g., by additional chemical moieties not normally part of the specific component). Such moieties could affect the physico chemical characteristic of the bile acid analog (i.e., solubility, absorption, half life and the like, decrease of toxicity). Such moieties are exemplified in “Remington: The Science and Practice of Pharmacy” by Alfonso R. Gennaro, 2003, 21st edition, Mack Publishing Company. Methods of coupling these chemical physical moieties to a bile acid are well known in the art.

In one embodiment, the bile acid or bile acid analogs of the present invention can comprise sodium deoxycholate (C₂₄H₃₉NaO₄), which is produced in the intestine from the salts of glycocholic and taurocholic acid by the action of bacterial enzymes. In another embodiment, the bile acid or bile acid analogs can comprise: cholic acid, chenodeoxycholic acid, taurocholic acid, taurochenodeoxycholic acid, glycocholic acid, glycochenocholic acid, 3β-monohydroxychloric acid, lithocholic acid, 3-hydroxy-12-ketocholic acid, 12-3-dihydrocholic acid, ursodesoxycholic acid, or any combination thereof.

In another one embodiment, the amount of bile acid or bile acid analog (e.g., sodium taurocholate, sodium deoxycholate) present in the oral combination therapies leptin formulations and compositions of the present invention is an amount to sufficiently allow intestinal absorption of the leptin or leptin functional derivative in the subject being administered. In another embodiment, the amount of bile acid or bile acid analog present in the oral combination therapies of the can range from about 1 mg/mL to about 25 mg/mL, about 1 mg/mL to about 12.5 mg/mL, or from about 5 to about 10 mg/mL. In another embodiment, the amount of bile acid or bile acid analog in the oral combination therapies of the present invention can vary from any one of about 1, 2, 3, 4, 5, 6, 7 mg/mL to any one of about 8, 9, 10, 11, 12, 12.5, . . . , 25 mg/mL. Other concentration ranges falling within 1 mg/mL and 25 mg/mL, which are not specifically recited here for brevity, are nevertheless included within the present invention. In another embodiment, the amount of bile acid or bile acid analog is about 30 mM.

In another embodiment, the above mentioned bile acids can be present in their soluble form or can be present as micelles without significantly affecting the ability of the orally administered leptin or leptin functional derivative to be absorbed or delivered to the bloodstream.

In another embodiment, the choice of bile acid or bile acid analog employed, or the form of the bile acid (e.g., soluble or micelle) within the compositions of the present invention can be made to optimize for example the kinetics of leptin delivery to the bloodstream, or the kinetics of leptin clearance from the bloodstream. Such optimizations would be within the capabilities of a person or ordinary skill in the art in view of the present invention.

In another embodiment, the agent capable of increasing leptin uptake by epithelial cells of the intestinal mucosa is an alcohol such as ethanol (CH₃CH₂OH). In one embodiment, the concentration of ethanol in the oral leptin formulation of the present invention is about 1% (v/v) to about 5% (v/v) or about 1% (v/v) to about 3% (v/v). In another embodiment, the concentration of ethanol in the oral leptin formulation of the present invention is about 2% (v/v) or about 3% (v/v).

Agents Enhancing Adherence to Treatment

In another aspect, the present oral combination therapies and compositions of the present invention can comprise an agent which is capable of enhancing treatment adherence such as a sweetener. In one embodiment, the sweetener is sucrose (C₁₂H₂₂O₁₁). In another embodiment, the sweetener is a nutritive sweetener (e.g., glucose, fructose (e.g., D-fructose), sugar cane, high fructose corn syrup (HFCS), agave syrup, honey and maple syrup) or a non-nutritive sweetener (e.g., aspartame, sucralose, and extracts from stevia rebaudiana). In another embodiment, the sweetener is present at a concentration of about 12 to 120 mg/mL. In another embodiment, the sweetener is present at a concentration of about 12 mg/mL. Other concentration ranges falling within 1 mg/mL and 120 mg/mL, which are not specifically recited here for brevity, are nevertheless included within the present invention.

Stimulators of Endogenous Leptin Secretion or Satiety Triggering Agents

In one embodiment, the oral combination therapies or compositions of the present invention can comprise a compound which is stimulator of endogenous leptin secretion. Such compounds can include: certain amino acids (e.g., glutamine); other peptide hormones (e.g., insulin, secretin, cholecystokinin (CCK), pentagastrin; steroid hormones (e.g., glucocorticoids); or transretinoic acids.

In another embodiment, the oral combination therapies or compositions of the present invention can comprise agents known to trigger satiety feelings in a subject. Such compounds can include peptides like glucagon-like peptide-1 (GLP-1) and peptide YY (PYY), or analogs thereof.

Preparations of the Oral Combination Therapies and Compositions

Oral combination therapies and compositions of the present invention can be prepared in the form of a liquid, (e.g., a syrup, a beverage) or a solid (e.g., a concentrate, a powder, a pill, a capsule or a tablet). Food products containing all of the compounds of the oral combination therapies of the present invention are also included such as a functional food, a food additive, a lozenge, a dragee, a confectionary, or a beverage. Other forms comprising the oral combination therapies of the present invention not specifically recited herein are nevertheless included.

In one embodiment, oral combination therapies and compositions of the present invention can be prepared in a liquid composition by dissolving appropriate amounts of the ingredients, other than the leptin (or the leptin functional derivative) and the protective agent (e.g., pancreatic protease inhibitors), in water and adjusting the pH with a base (e.g., NaOH) to obtain a stock solution with basic pH. In one embodiment, the pH is adjusted to between about pH 7 to about pH 11. In another embodiment, the pH can be adjusted to about pH 7, 8, 9, 10 or 11. The stock solution can then be refrigerated. Vehicle solutions can be prepared from the stock solutions by dissolving the protective agent (e.g., the pancreatic protease inhibitors) in an appropriate amount of stock solution. The vehicle solution can then be used to dissolve the desired amount of leptin or leptin functional derivative in order to obtain an oral combination therapy of the present invention in liquid form.

Methods and Uses

In another aspect, the present invention relates to a method for the oral administration of leptin in a subject, said method comprising administering to the subject a oral combination therapy or composition as defined herein, wherein the leptin or leptin functional derivative is delivered to the subject's bloodstream in an active form thereof. Without being bound by theory, the oral combination therapies and compositions of the present invention protect the leptin or leptin functional derivative in the gastrointestinal tract so that it can bind to an intestinal leptin receptor expressed by duodenal cells. The leptin or leptin functional derivative is then absorbed by the duodenal cells and released into the bloodstream bound to a soluble leptin receptor produced by the same duodenal cells. Thus, it is a complex of leptin (or leptin functional derivative) bound to a soluble leptin receptor that reaches the blood. This complex is much more stable and remains for longer periods of time in the bloodstream compared to free leptin (i.e., unbound to its soluble receptor). The complex then reaches the central nervous system and interacts with its target cells in a physiological manner to regulate appetite, body weight, and/or energy metabolism in the subject. Although the majority of the leptin (or leptin functional derivative) is though to be internalized by duodenal cells via leptin receptor, it is possible that other mechanisms independent of the leptin receptor exist whereby orally administered leptin can reach the bloodstream. The present invention encompasses these other mechanisms as well.

In one embodiment, the present invention relates to a method for the oral administration of leptin (or a leptin functional derivative) in a subject for preventing, treating and/or managing a disease, condition or phenotype that is associated with low plasma leptin levels or that can be ameliorated by increasing plasma leptin levels; or for the manufacture of an oral combination therapy for accomplishing same. In another embodiment, the above mentioned disease, condition or phenotype includes: obesity, type 1 diabetes, type 2 diabetes, hypothalamic amenorrhea, cardiovascular diseases, depression, a hypoleptinemic disease, a leptin deficient state, weight gain, or a condition that can be ameliorated by weight loss or by an increase in the levels of plasma leptin.

In another embodiment, the present invention relates the use of orally administered leptin (or a leptin functional derivative) for controlling/managing: appetite; body weight; rate of weight gain or loss; and/or energy usage/metabolism. In another embodiment, the present invention relates the use of orally administered leptin (or a leptin functional derivative) by otherwise healthy subjects as a supplement or food additive, used either regularly or sporadically.

In another embodiment, the oral combination therapies of the present invention is eligible for natural health product status. “Natural health product” (or “health-promoting agent”, “health-enhancing agent”, or “health product”) as used herein refers to a substance or combinations of substances found in nature or energetically potentiated preparations that are used for the purpose of maintaining or improving health, or treating or preventing disease conditions. These compounds generally include, but are not limited to, vitamins, minerals, enzymes, co-enzymes, co-factors, herbs or botanicals, naturally occurring animals, plant and microorganism substances, and a variety of molecules extracted from natural sources such as amino acids, polysaccharides, peptides, naturally occurring hormones and biochemical intermediates, as well as naturally occurring molecules synthesized by chemical or biological means.

In another embodiment, the oral combination therapies of the present invention is a nutraceutical. “Nutraceutical” as used herein generally includes to a food or food product that provides health and medical benefits, including the prevention and treatment of disease. “Nutraceutical” can also include a product isolated or purified from foods that is generally sold in medicinal forms not usually associated with food. A nutraceutical is generally demonstrated to have a physiological benefit or provide protection against chronic disease.

The present invention is illustrated in further details by the following non-limiting examples.

### Example 1

### Preparation of Oral Leptin Formulations of the Present Invention

Two oral combination therapies of the present invention in liquid form (hereinafter referred to as “leptin formulations”) were prepared and tested by dissolving murine leptin in a solution of either Vehicle 1 or Vehicle 2, as detailed below. Unless otherwise indicated, “leptin” as used in the present Examples refers to murine leptin having SEQ ID NO: 1 as shown below. Sodium bicarbonate, sodium deoxycholate, sucrose and ethanol were obtained from Sigma-Aldrich. The “anti-protease mix” was obtained from Roche Diagnostics (Complete™, Mini, EDTA-free, Protease Inhibitor Cocktail Tablets; Cat. No. 11 836 170 001). Recombinant mouse leptin was obtained from R & D Systems and had the following amino acid sequence:

(SEQ ID NO: 1)
MVPIQKVQDDTKTLIKTIVTRINDISHTQSVSAKQRVTGLDFIPGLHPIL
SLSKMDQTLAVYQULTSLPSQNVLQIANDLENLRDLLHLLAFSKSCSLPQ
TSGLQKPESLDGVLEASLYSTEVVALSRLQGSLODILQQLDVSPEC

The above sequence differs from that of native murine leptin by the addition of a single methionine residue (underlined) at the N terminus of the native protein by the manufacturer (R & D systems).

| Vehicle 1 | Vehicle 2 |
|---|---|
| NaHCO₃ (125 mM) | NaHCO₃ (125 mM) |
| Commercial anti-protease | Commercial anti-protease |
| mix (1 tablet for 10 mL) | mix (1 tablet for 10 mL) |
| | Sodium deoxycholate (30 mM) |
| | Ethanol (3%) |
| | Sucrose (120 g/L) |

A 100 mL stock solution of Vehicle 1 was prepared by dissolving 1.05 g of NaHCO₃ in 80 mL of distilled water. The mixture was stirred until complete dissolution of all compounds. The pH of the solution was adjusted to 9 using NaOH (10 N and 1N). The volume of the solution was then adjusted to 100 mL and the solution was kept at 4° C.

A 100 mL stock solution of Vehicle 2 was prepared by dissolving 1.05 g of NaHCO₃, 1.24 g of sodium deoxycholate, and 1.2 g of sucrose in 80 mL of distilled water. 3 mL of ethanol 100% (pure) was then added and the mixture was stirred until complete dissolution of all compounds. The pH of the solution was adjusted to 9 using NaOH (10 N and 1 N). The volume of the solution was then adjusted to 100 mL and the solution was kept at 4° C.

On the day of an experiment, one tablet of the anti-protease mix was dissolved in 10 mL of stock solution (i.e., vehicle 1 or 2). This working solution, in which leptin was dissolved, was kept up to five days at 4° C.

### Example 2

### Assays to Measure Mouse, Rat and Human Leptin Levels

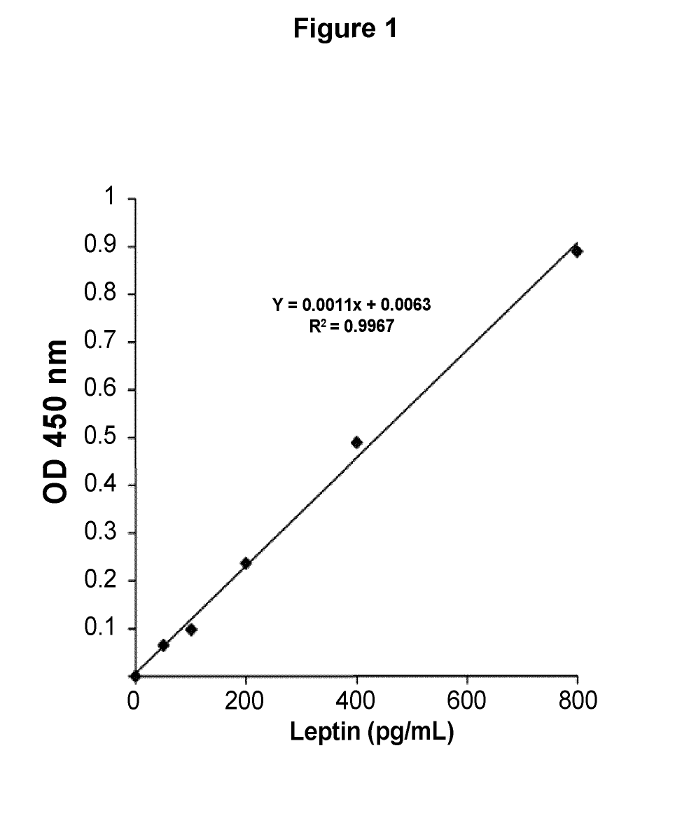

Plasma mouse leptin and in vitro mouse leptin levels in a simulated gastric environment were measured using a mouse leptin enzyme immunoassay (EIA) kit (product no. ADI-900-19A) from Enzo Life Science according to the instructions from the manufacturer. FIG. 1 shows an exemplary standard curve for leptin as measured by the above enzyme immunoassay using the leptin standard provided with the EIA kit. This leptin standard (3200 pg/mL) was diluted as recommended in the kit assay buffer to reach a concentration of 50 pg/mL. As shown in the figure, the standard curve for leptin was generally linear between leptin concentrations of 0 to 800 pg/mL.

Plasma human leptin and in vitro human leptin levels in a simulated gastric or duodenal environment were determined using a Quantikine™ leptin immunoassay kit (R&D Systems, Inc., USA; catalog No. DLP00) according to the instructions from the manufacturer, unless otherwise indicated.

Plasma rat leptin levels were determined using a Quantikine™ leptin immunoassay kit (R&D Systems, Inc., USA; catalog No. MOB00) according to the instructions from the manufacturer, unless otherwise indicated.

### Example 3

### Effect of Oral Leptin Formulations of the Present Invention on Plasma Leptin Levels

In normal physiological conditions, leptin is secreted by the gastric mucosa in an exocrine way into the gastric juice. It is then absorbed by the intestinal mucosa to reach the bloodstream. The present assay sought to determine whether leptin administered orally follows the same path.

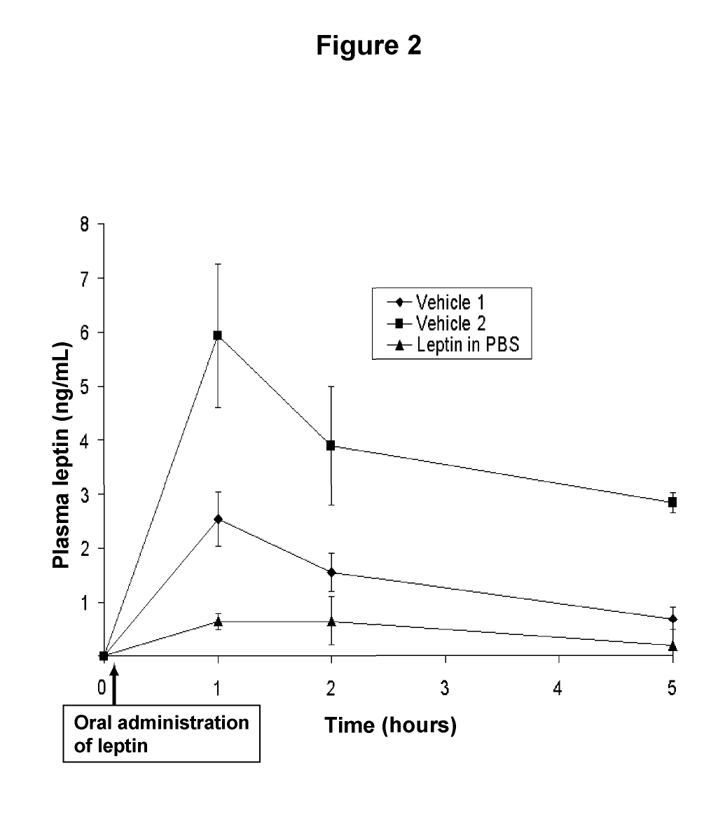

Overweight C57BL/6J ob/ob mice 5-8 weeks old, obtained from Jackson Laboratories (Bar Harbor, Me., USA), were administered oral leptin formulations prepared as described in Example 1 and plasma leptin levels were measured as described in Example 2. These mice were chosen because they have a genetic deficiency that renders them leptin-deficient, and therefore any appearance of leptin in the blood must originate from the orally administered leptin. Briefly, five ob/ob mice were force-fed with 50 μg of leptin formulated in vehicle solution 1, vehicle solution 2, or in phosphate buffer (PBS) using a cannula. Blood was sampled before administration of leptin in vehicle 1 (FIG. 2, diamonds), leptin in vehicle 2 (FIG. 2, squares), or leptin in PBS (FIG. 2, triangles) as well as at 1, 2 and 5 hours post-administration. Blood leptin levels were determined as described in Example 2.

Prior to the oral administration of leptin in vehicles 1, 2, or leptin in PBS, plasma leptin was undetectable, confirming the absence of leptin in these genetically deficient animals. As shown in FIG. 2, plasma leptin levels rose after 1 hour post-administration for leptin in vehicles 1 and 2, and then more slowly decreased. leptin in vehicle 2 (squares) resulted in higher plasma leptin levels than leptin in vehicle 1 (diamonds). Higher plasma leptin level denotes a better protection of leptin by the vehicle and/or absorption of leptin. Interestingly, plasma leptin levels were detected in a range corresponding to physiological concentrations normally observed in wild-type C57BL/6J mice.

### Example 4

### Effect of Different Doses of Oral Leptin on Body Weight of ob/ob Mice

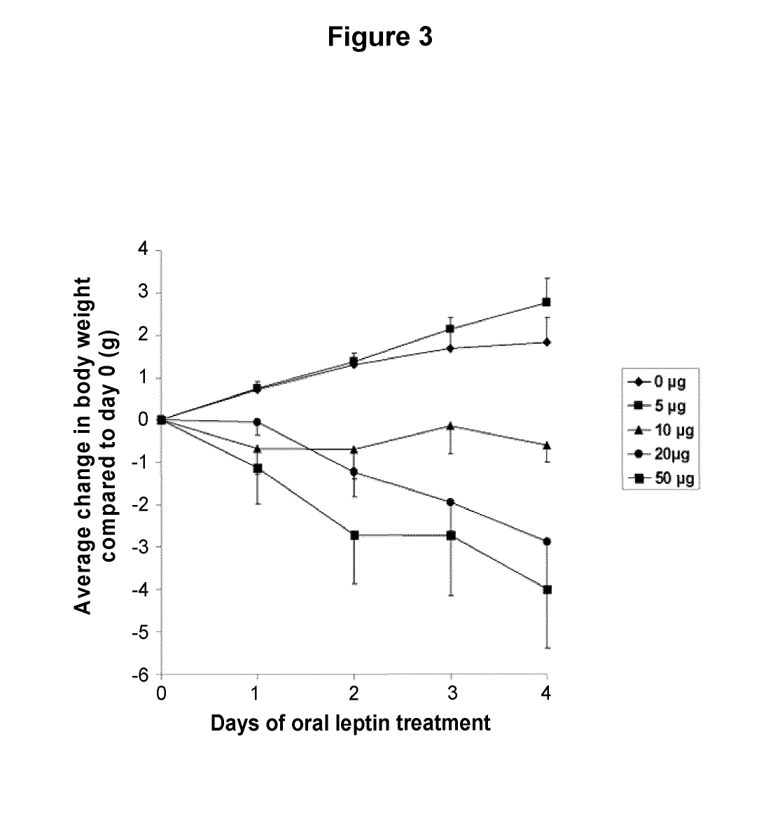

Five ob/ob mice were orally administered leptin formulations containing different amounts of leptin formulated in vehicle solution 2, which were prepared as described in Example 1. Administrations were performed twice a day for four consecutive days (FIG. 3, “Day 0” to “Day 3”) during mornings (about 8 AM) and evenings (about 6 PM). Oral leptin formulations in vehicle solution 2 containing five different amounts of leptin were tested: 0, 5, 10, 20 and 50 μg (FIG. 3: diamonds, upper squares, triangles, circles and lower squares, respectively). The body weight of the mice was measured during the mornings. The results shown in FIG. 3 are expressed as the change in body weight of the mice (i.e., loss or gain in grams) compared to their initial weight before leptin treatment as a function of time (in days).

As shown in FIG. 3, mice receiving vehicle solution 2 alone (0 μg of leptin, diamonds) continued to gain weight throughout the duration of the study. The change in body weight of mice receiving 5 μg of leptin formulated in vehicle solution 2 (upper squares) was not significantly different from change in body weight of mice treated with vehicle 2 alone (diamonds). In contrast, mice receiving higher amounts of leptin maintained or reduced their body weight. More particularly, the body weights of mice receiving 10 μg of leptin formulated in vehicle 2 (triangles) remained generally stable over the duration of the study. Mice receiving 20 μg and 50 μg of leptin formulated in vehicle solution 2 (circles and lower squares, respectively) significantly reduced their body weight in a dose-dependent fashion over the course of the study.

### Example 5

### Effect of Oral Leptin Formulations of the Present Invention on Food Intake and Body Weight in ob/ob Mice

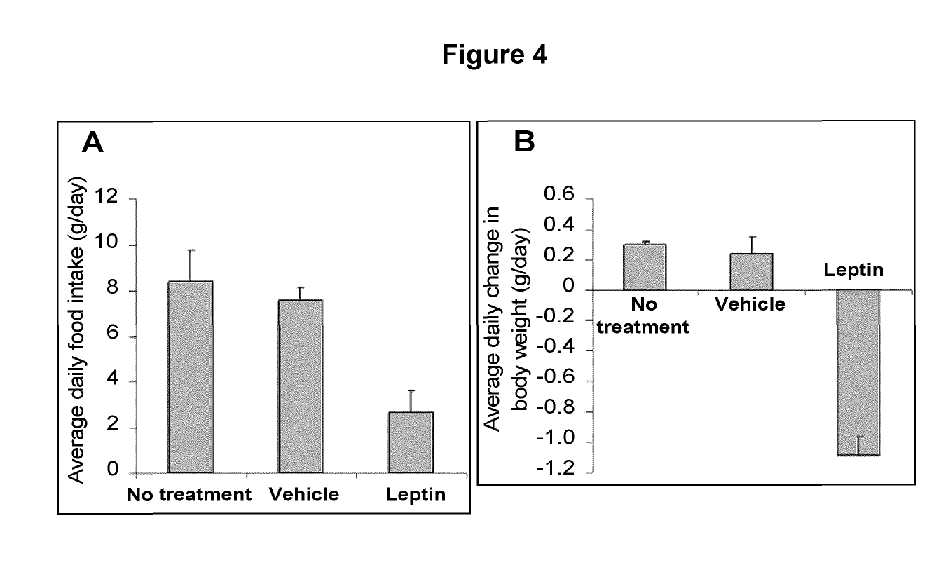

Three groups of five overweight ob/ob mice 5-8 week old were orally administered leptin formulations containing 50 μg of leptin formulated in vehicle solution 2, vehicle solution 2 alone, or no treatment, mornings and evenings as described in Example 3. Food intake and body weight were measured daily for the mice for four consecutive days and, at the end of this period, the average daily food intake (in grams per day, FIG. 4A) and the average daily change in body weight (in grams per day, FIG. 4B) were calculated. As shown in FIG. 4A, mice receiving vehicle solution 2 alone (i.e., without leptin, “vehicle”) ate similar amounts of food compared to mice receiving no treatment (“no treatment”). However, mice receiving the oral leptin formulation (“leptin”) ate an average of about 65% less food than the ones receiving vehicle solution 2 alone (“vehicle”). As shown in FIG. 4B, the body weight of the mice receiving no treatment (“no treatment”) increased regularly by an average of about 0.3 g/day. Vehicle-treated mice (“vehicle”) displayed a similar rate of average weight gain as the mice receiving no treatment. In contrast, mice receiving the oral leptin formulation lost an average of more than 1 g/day.

### Example 6

### Effect of Long-Term Administration of Oral Leptin Formulations of the Present Invention on ob/ob Mice

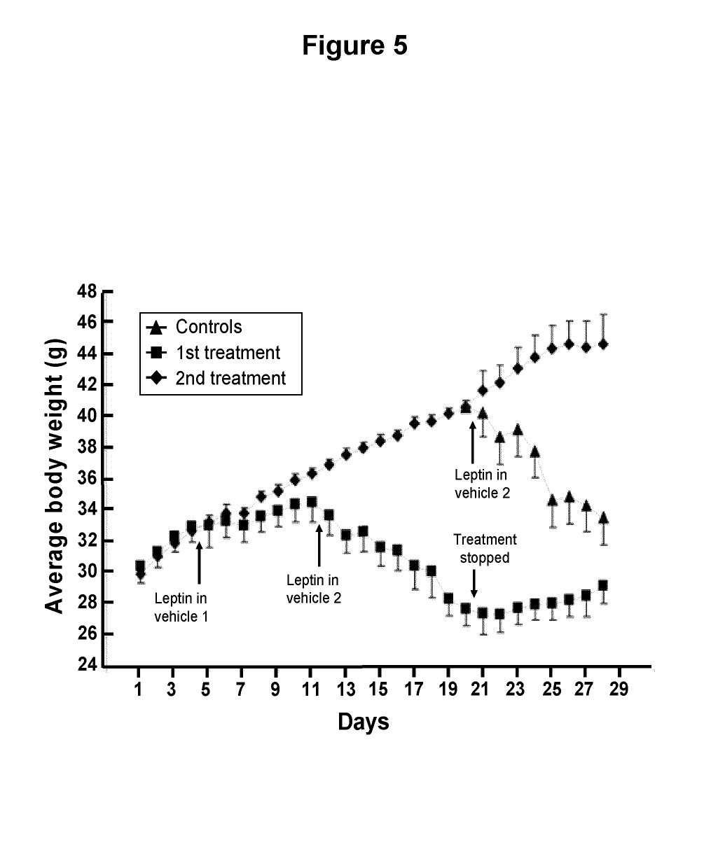

Fifteen overweight ob/ob mice (5-6 weeks old) having an average weight of about 30 g were allowed unlimited access to food and water for four consecutive days (FIG. 5, “Day 0” to “Day 4”). The mice were then separated into two groups. The first group consisted of ten mice which were orally administered vehicle solution 1 without leptin (FIG. 5, diamonds, “Day 5”). The second group consisted of five mice orally administered 50 μg of leptin formulated in vehicle solution 1 (FIG. 5, squares, “Day 5”). Administrations were performed twice a day, mornings (about 8 AM) and evenings (about 6 PM) and body weights were measured once a day (at about 8 AM). After seven days of daily treatments (FIG. 5, “Day 11”), the leptin-treated group (squares) had an average weight of 34.6±1.25 g (n=5) while the vehicle-treated group (diamonds) had an average weight of 36.5±0.38 g (n=10).

At this stage, vehicle solution 1 was replaced by vehicle solution 2 for both groups of mice (FIG. 5, “Day 12”). After ten days of daily treatments (FIG. 5, “Day 20”), the vehicle-treated group (diamonds) continued to gain weight to reach an obese state having an average body weight of 41.83±0.50 g (n=10) while the average body weight of the leptin-treated group (squares) decreased to 27.4±1.07 g (n=5). These results demonstrate that long-term administration of oral leptin formulations of the present invention, particularly that containing vehicle solution 2, potently decrease body weight.

At this stage, daily leptin treatments of the leptin-treated group were stopped (FIG. 5, “Day 21”) and this group of five mice (squares) slowly began to gain weight again, confirming the role of leptin in the control of body weight as well as the reversible nature of the effect of oral leptin formulations of the present invention.

In parallel, in order to assess the effect of oral leptin formulations of the present invention on obese animals, the ten obese mice (FIG. 5, diamonds, “Day 21”) were at that time subdivided into: (a) a vehicle-treated group (n=5) which continued to receive daily oral administrations of vehicle solution 2 alone (without leptin) (diamonds, “Day 21” to “Day 28”); and (b) a leptin-treated group (n=5) receiving daily oral administrations of 50 μg of leptin formulated in vehicle solution 2 (triangles, “Day 21” to “Day 28”). After eight days of daily treatments, the vehicle-treated group continued to gain weight and reached an average weight of 44.8±1.89 g (n=5) (diamonds, “Day 28”). In contrast, the average body weight of the leptin-treated mice decreased to 33.7±1.12 g (n=5) (triangles, “Day 28”). These striking results demonstrate that oral leptin formulations of the present invention can induce weight loss in both obese and overweight but not non-obese ob/ob mice.

### Example 7

### Effect of Oral Leptin Formulations of the Present Invention on Food Intake and Body Weight in Normal, Non-Obese Wild-Type C57BL/6J Mice

To study the effect of oral leptin administration in normal mice, the inventors choose to work with C57BL/6J mice which are the non-obese genetic equivalent of the ob/ob mice. Normal wild-type C57BL/6J mice are able to synthesize leptin and leptin receptor, and are normoleptinemic (i.e., they are able to attain normal levels of plasma leptin). When healthy and kept on a diet of standard Purina chow, these mice remain sensitive to leptin and generally maintain a lean body weight.

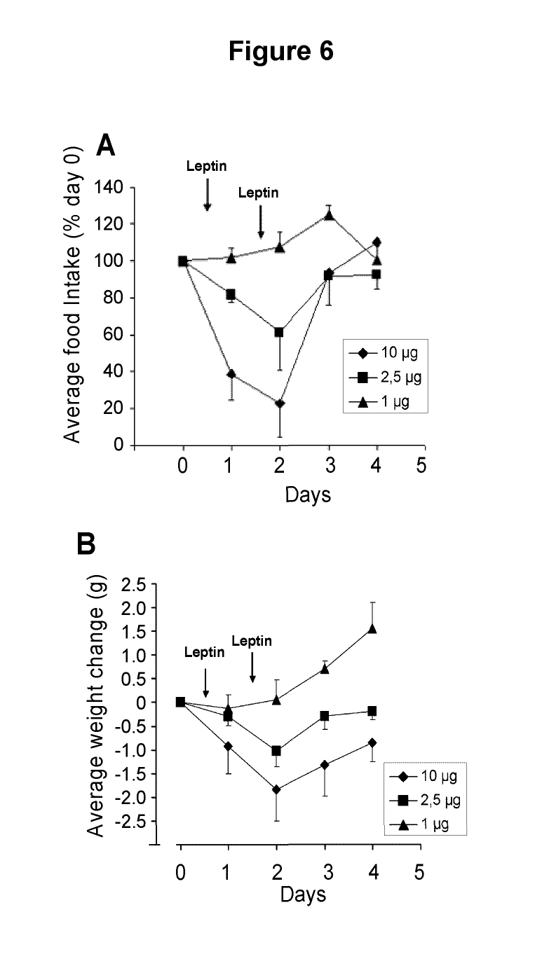

Five overweight C57BL/6J mice 5-8 weeks old, obtained from Jackson Laboratories (Bar Harbor, Me., USA) were force-fed using a cannula 1, 2.5 and 10 μg of mouse leptin formulated in vehicle solution 2 (FIG. 6, triangles, squares and diamonds, respectively) as described in Example 1 for two consecutive days (shown with arrows on Figure). Blood was sampled before administration of leptin formulations and after 30 minutes, 1 hour and 2 hours post-administration. Plasma leptin levels were measured as described in Example 2.

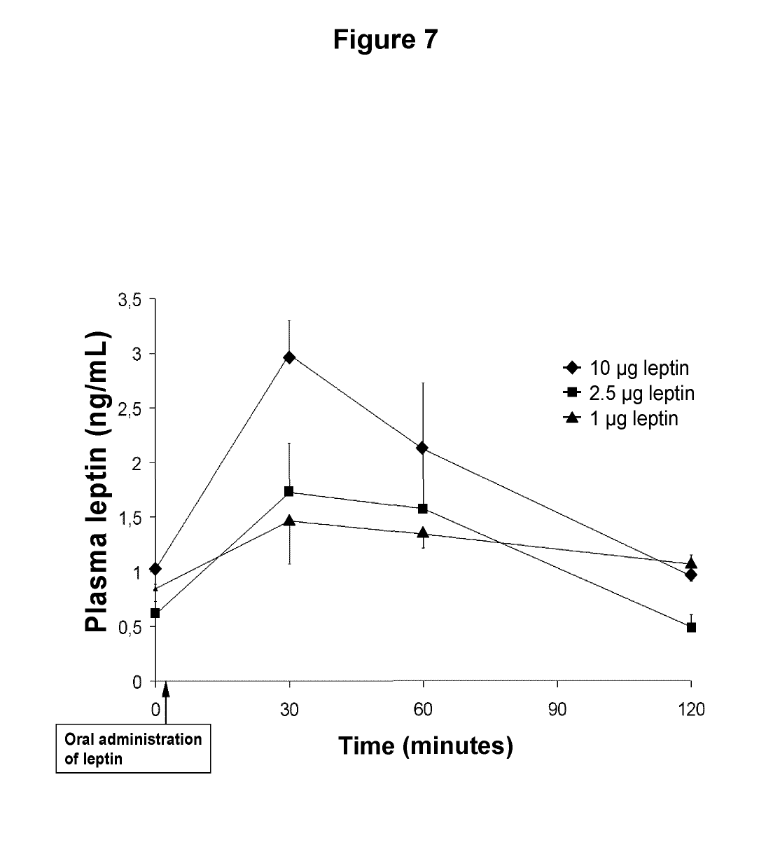

As shown in FIG. 6, the leptin-administered mice immediately and dose-dependently reacted to the oral leptin formulations of the present invention in terms of reduced food intake and body weight loss. Furthermore, withdrawal of leptin administration immediately reversed the anorectic effect. Plasma leptin levels after oral administration of the leptin formulation of these mice is shown in FIG. 7. As seen in FIGS. 6 and 7, while 1 μg of oral leptin was too low to trigger any response, 2.5 μg of oral leptin resulted in weight loss (FIG. 6). This confirms that C57BL/6J mice are more sensitive to leptin than the ob/ob mice.

Comparison of Various Oral Leptin Formulations

In order to determine the effect of each component of the vehicle 2 formulation (sodium bicarbonate 125 mM; sodium deoxycholate 30 mM; anti-proteases mix (Roche Diagnostics™) 1 tablet/10 mL; sucrose 120 g/L; ethanol 3%) formulation, a series of experiments was carried out by removing either the vehicle itself or one element of the vehicle at a time. In Examples 8 to 12, the formulations were administered by force feeding wild-type C57BL/6J mice and measuring plasma leptin levels, as described in Example 7.

### Example 8

### Effect of Vehicle 2 Alone or the Administration Method Per Se (in the Absence of Leptin) on Plasma Leptin Levels in Wild-Type C57BL/6J Mice

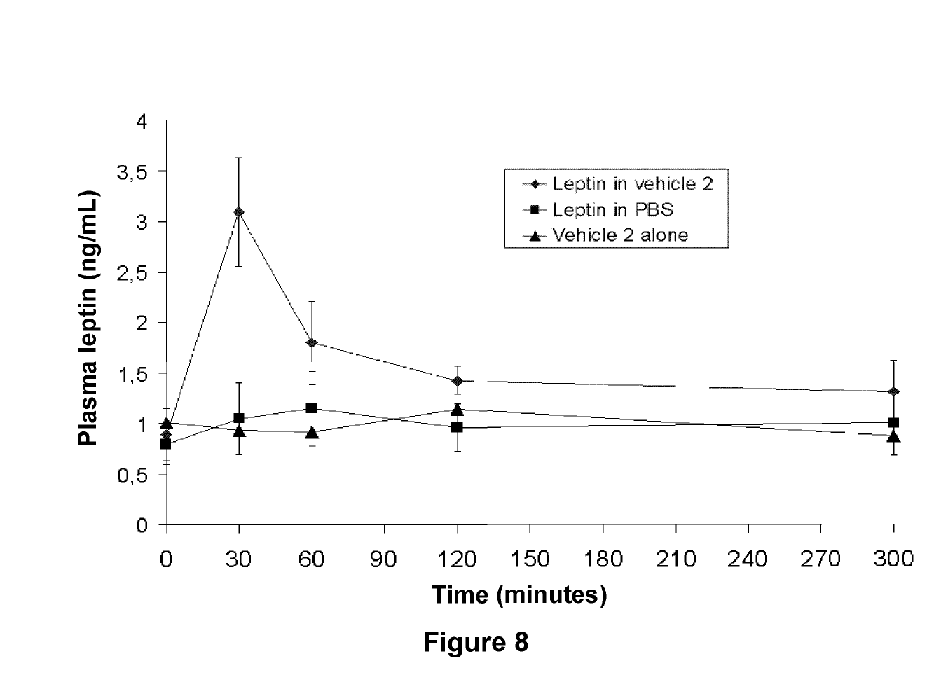

To address the possibility that the vehicle by itself may be responsible for the anorexic effect of orally administered leptin, or that handling the animals may cause a severe stress leading to decrease in food intake, wild-type C57BL/6J mice were administered: 10 μg of leptin in vehicle 2 (FIG. 8, diamonds); 10 μg of leptin in PBS (FIG. 8, squares); or vehicle 2 alone without leptin (FIG. 8, triangles). These results show that the administration technique (i.e., force-feeding) or the vehicle 2 alone (in the absence of leptin) has no significant effect on plasma leptin levels in wild-type C57BL/6J mice.

### Example 9

### Effect of Removal of Bicarbonate Buffer on Plasma Leptin Levels in Wild-Type C57BL/6J Mice

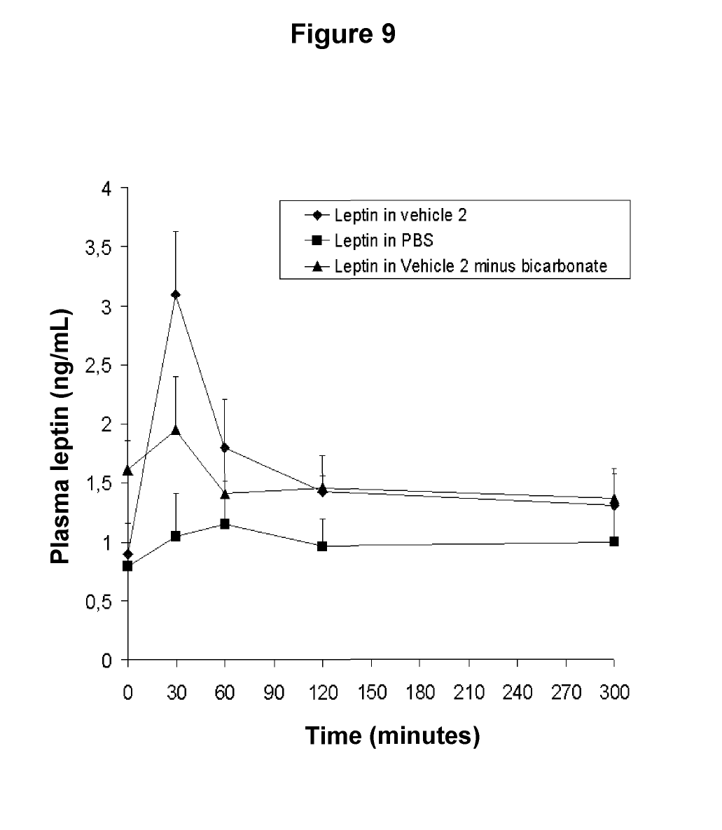

The bicarbonate buffer was removed from vehicle 2 and six wild-type C57BL/6J mice were force-fed with the modified vehicle 2 (i.e., without sodium bicarbonate) containing 10 μg of leptin. The results in FIG. 9 show that buffering with bicarbonate buffer results in a significant increase in plasma leptin levels.

### Example 10

### Effect of Removal of Bile Salt on Plasma Leptin Levels in Wild-Type C57BL/6J Mice

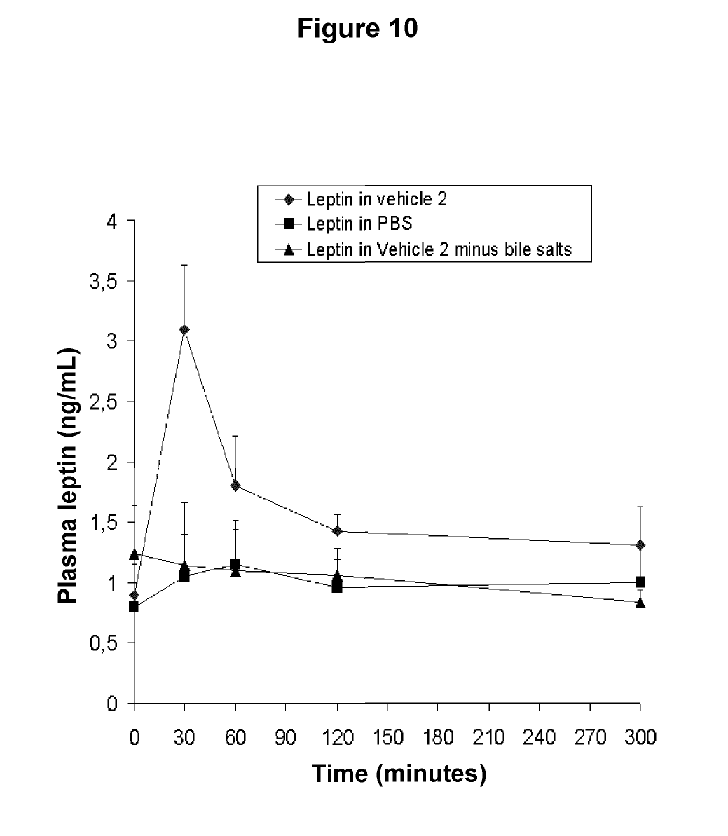

The bile salt was removed from vehicle 2 and six wild-type C57BL/6J mice were force-fed the modified vehicle 2 (i.e., without sodium deoxycholate) containing 10 μg of leptin. The results in FIG. 10 show that the bile salt produces a detectable increase in plasma leptin levels.

### Example 11

### Effect of Removal of Anti-Protease Mix on Plasma Leptin Levels in Wild-Type C57BL/6J Mice

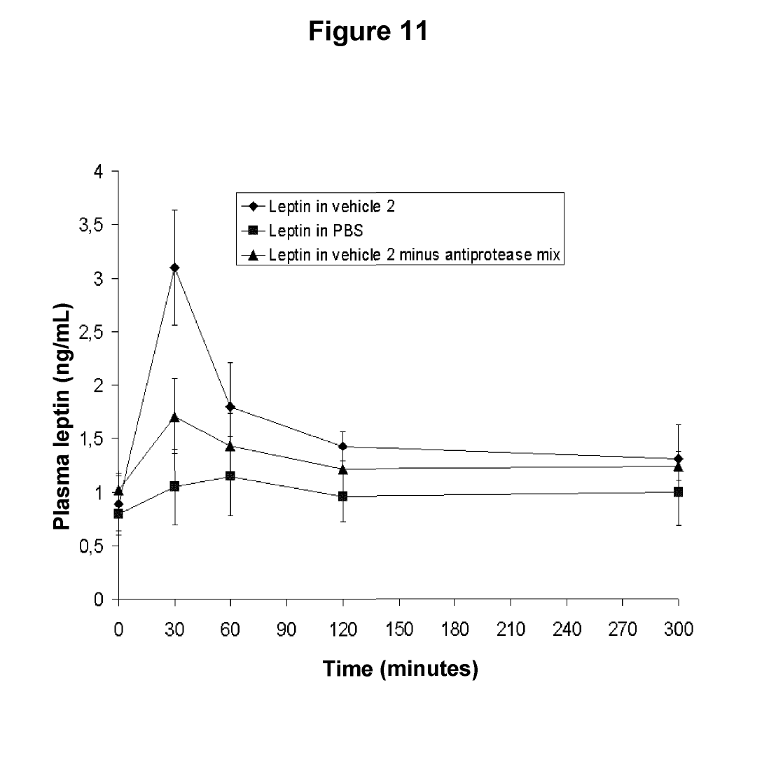

The anti-protease mix was removed from vehicle 2 and six wild-type C57BL/6J mice were force-fed the modified vehicle 2 (i.e., without the anti-protease mix) containing 10 μg of leptin. The results in FIG. 11 show that the presence of the anti-protease mix results in a significant increase in plasma leptin levels.

### Example 12

### Effect of Removal of Ethanol on Plasma Leptin Levels in Wild-Type C57BL/6J Mice

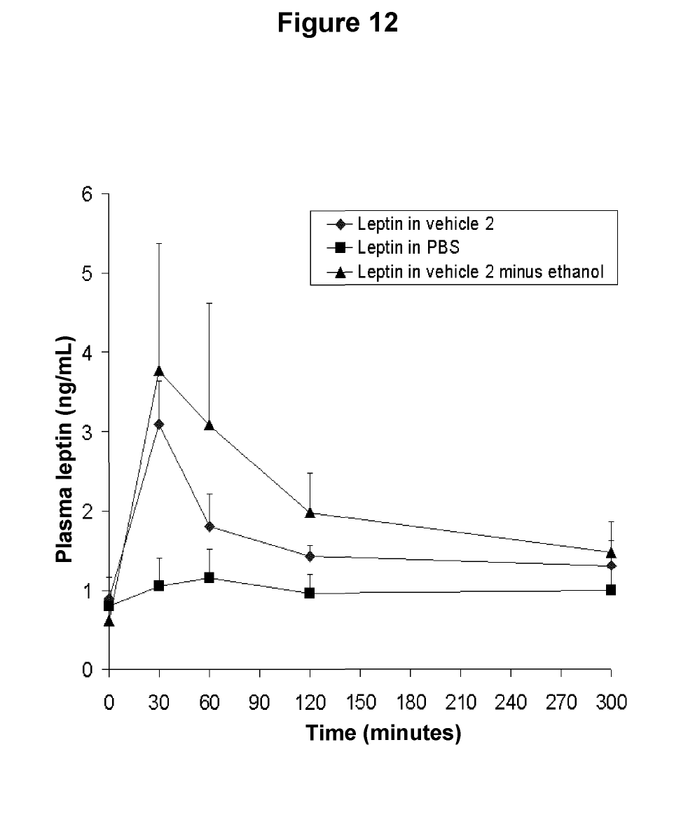

The ethanol was removed from vehicle 2 and six wild-type C57BL/6J mice were force-fed the modified vehicle 2 (i.e., without ethanol) containing 10 μg of leptin. The results in FIG. 12 show that higher plasma leptin levels was observed for the leptin formulation lacking ethanol.

### Example 13

### Effect of Removal of Sucrose on Plasma Leptin Levels in Wild-Type C57BL/6J Mice

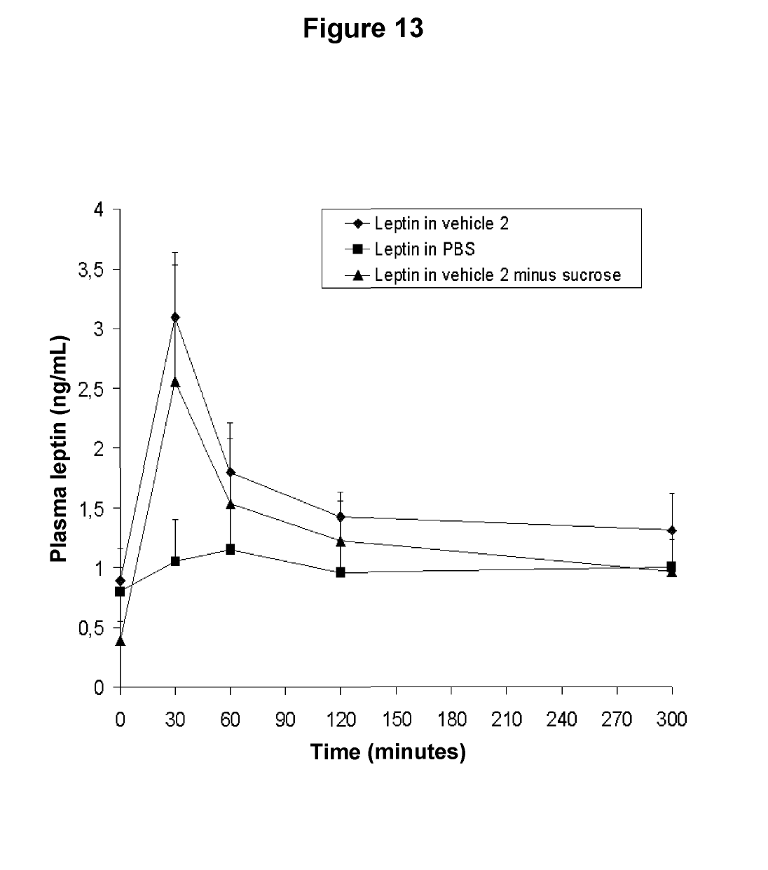

The sucrose was removed from vehicle 2 and six wild-type C57BL/6J mice were force-fed the modified vehicle 2 (i.e., without sucrose) containing 10 μg of leptin. The results in FIG. 13 show that the removal of sucrose does not significantly affect plasma leptin levels.

### Example 14

### Preparation of Oral Leptin Formulation Using Vehicle 3

A Vehicle 3 having the following composition was prepared:

VEHICLE 3:

Sodium bicarbonate (NaHCO₃) (125 mM)
Sodium deoxycholate (30 mM)
Commercial anti-protease mix (1 tablet per 10 mL), pH 9.

A 100 mL stock solution of Vehicle 3 was prepared by dissolving 1.05 g of NaHCO₃, 1.24 g of sodium deoxycholate, in 80 mL of distilled water. The mixture was stirred until complete dissolution of all compounds. The pH of the solution was adjusted to 9 using NaOH (10 N and 1N). The volume of the solution was then adjusted to 100 mL and the solution was kept at 4° C.

On the day of an experiment, one tablet of the anti-protease mix was dissolved in 10 mL of stock solution of Vehicle 3. This working solution, in which leptin was dissolved (at a concentration of 10 μg leptin/100 μL stock solution of vehicle 3, unless otherwise indicated), was kept up to five days at 4° C. The volumes of the vehicle (with leptin) administered varied according to the final amount of leptin to be administered.

A series of experiments were carried out by substituting and/or removing one or more components of the vehicle 3 formulation. The formulations were administered by force feeding wild-type C57BL/6J mice and measuring plasma leptin levels, as described in Example 7.

### Example 15

### Effect of Different Bile Acids on Plasma Leptin Levels in Wild-Type C57BL/6J Mice

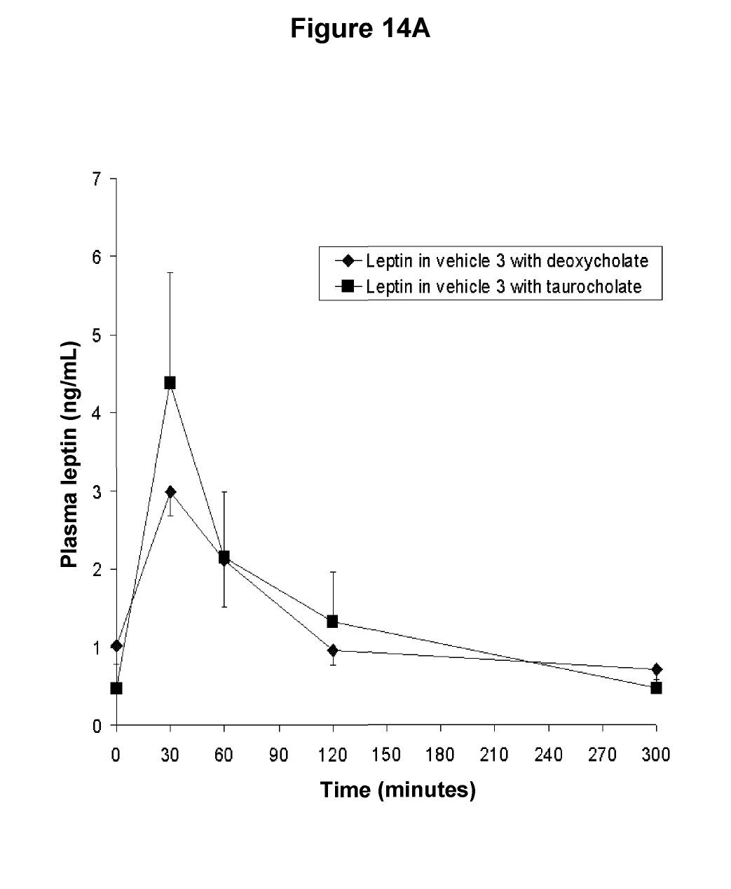

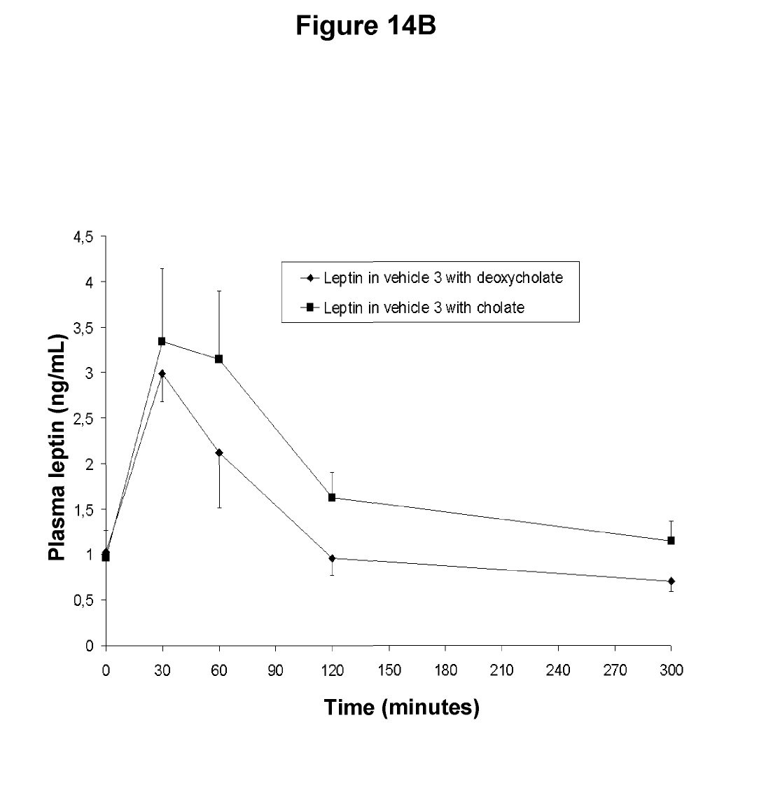

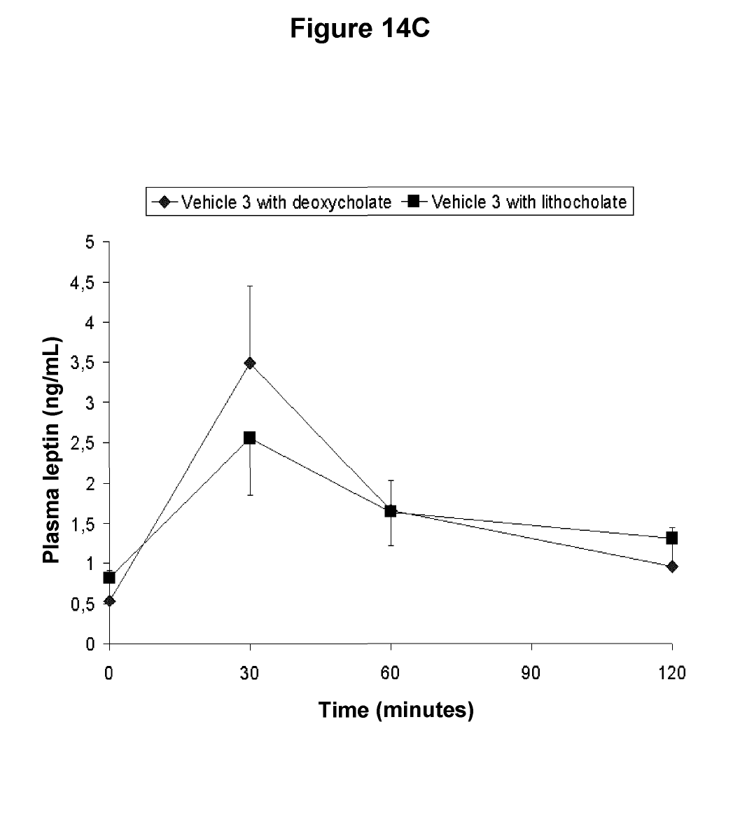

The sodium deoxycholate in the vehicle 3 formulation was substituted with other bile acids (i.e., taurocholate; cholate; lithocholate) and the results were compared with that of sodium deoxycholate. FIG. 14, panels A, B and C show the results comparing taurocholate, cholate, and lithocholate, respectively, with sodium deoxycholate. As can be seen in these figures, taurocholate was significantly more efficient than sodium deoxycholate in increasing leptin absorption (FIG. 14A). Cholate also showed higher efficiency in leptin absorption when compared to sodium deoxycholate (FIG. 14B). Lastly, lithocholate resulted in only slightly lower plasma leptin levels than sodium deoxycholate (FIG. 14C). Collectively, these results show that the oral leptin formulations are effective using different bile acids.

### Example 16

### Comparison of Bile Acids Present in Soluble or Micelle Form on Plasma Leptin Levels in Wild-Type C57BL/6J Mice

Taurocholate can be obtained either in soluble form or in the form of micelles, after being mixed with cholesterol and fatty acids. This micelle form of bile salts was then tested in the context of oral leptin administration. The soluble taurocholate in the oral leptin formulation described in Example 13 was replaced with taurocholate in the form of micelles (30 mM) according to standard laboratory techniques. Briefly, the micelle form of taurocholate was prepared by mixing 35 μL of linolenic acid (1M), 64 mg of taurocholate, and 4.2 mg of cholesterol in 5 ml of NaCl (0.8 g/l). The mixture was allowed to dry for 3-4 hours under gentle heat, and then reconstituted in NaOHCO₃ (125 mM) and pH is adjusted to pH 9.

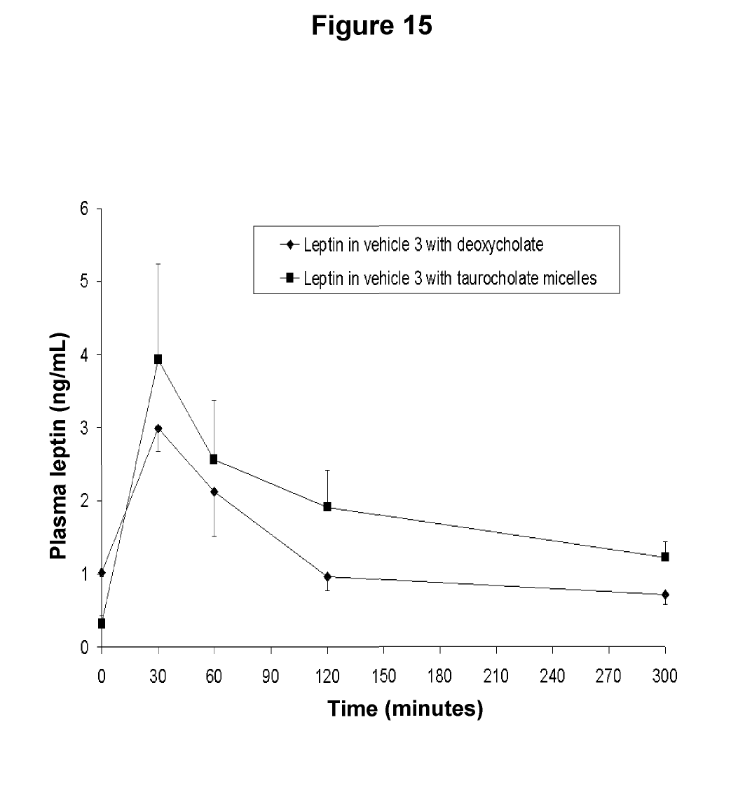

A comparison of the results in FIG. 15 with those of FIG. 14A, and other results, show that the form of taurocholate (soluble versus micelle) in the oral leptin formulation does not appear to greatly affect the absorption efficiency of oral leptin.

### Example 17

### Comparison Between a Commercially Obtained Anti-Protease Mix and a Homemade Mix of Protease Inhibitors on Plasma Leptin Levels in Wild-Type C57BL/6J Mice

In order to better characterize the effect of the anti-protease mix in the vehicle 3 formulation, two approaches were investigated.

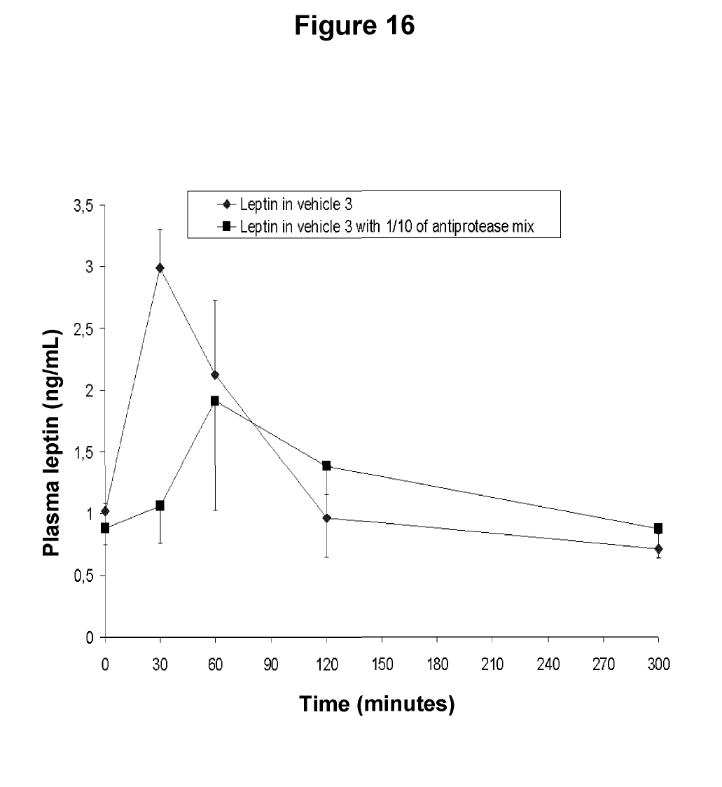

In the first approach, the concentrations of the commercial anti-protease mix obtained from Roche Diagnostics™ were decreased. Normal C57BL/6J mice were administered vehicle 3 containing 10 μg leptin by force-feeding except with 1/10 of the original amount of the anti-protease mix. The results are shown in FIG. 16. As can be seen, reducing the amounts of anti-proteases by ten-fold reduced plasma leptin levels.

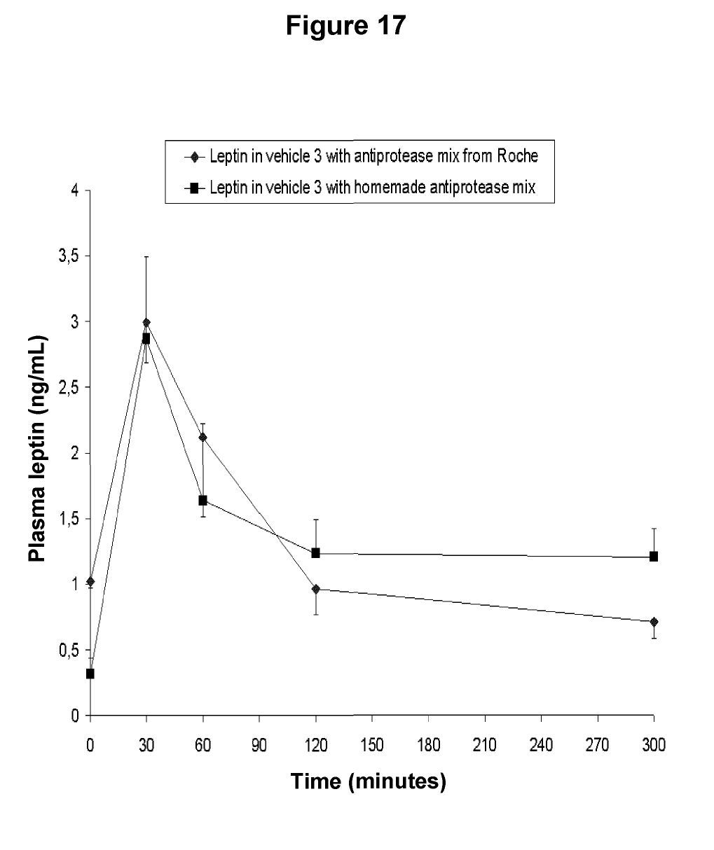

In the second approach, the commercial anti-protease mix from Roche Diagnostics™ was replaced with another mix of anti-proteases/protease inhibitors (used interchangeably hereafter) prepared by the inventors. The following mixture and concentrations of anti-proteases were used: Aprotinin 10 μg/mL; alpha2-macroglobulin 1 μg/mL; leupeptin 10 μg/mL; chymostatin 10 μg/mL; trypsin inhibitor 10 μg/mL; and PMSF 20 μg/mL. FIG. 17 shows a comparison between the commercial anti-proteases mix from Roche Diagnostics™ or the homemade mix of protease inhibitors. The results show that the homemade mix of protease inhibitors had substantially the same protective effect as the commercial anti-protease mix.

### Example 18

### Preparation of Oral Leptin Formulation Using Vehicle 4

A vehicle 4 having the following composition was prepared:

VEHICLE 4:

Sodium bicarbonate, 125 mM
Sodium taurocholate, 30 mM
Aprotinin, 10 μg/mL
Alpha2-macroglobulin, 1 μg/mL
Leupeptin, 10 μg/mL
Chymostatin, 10 μg/mL
Trypsin inhibitor, 10 μg/mL
PMSF 20 μg/mL

A 100 mL stock solution of Vehicle 4 was prepared by dissolving 1.05 g of NaHCO₃, 1.24 g of sodium taurocholate, in 80 mL of distilled water. The mixture was stirred until complete dissolution of all compounds. The pH of the solution was adjusted to 9 using NaOH (10 N and 1N). The volume of the solution was then adjusted to 100 mL and the solution was kept at 4° C.

On the day of an experiment, Aprotinin 10 μg/mL; alpha2-macroglobulin 1 μg/mL; leupeptin 10 μg/mL; chymostatin 10 μg/mL; trypsin inhibitor 10 μg/mL; and PMSF 20 μg/mL were dissolved in 10 mL of stock solution of Vehicle 4. This working solution, in which leptin was dissolved, was kept up to five days at 4° C.

### Example 19

### Effect of Different Buffers on Plasma Leptin Levels in Wild-Type C57BL/6J Mice

Vehicle 3 was modified by substituting sodium bicarbonate (NaHCO₃) with e.g., either phosphate (e.g., NaHPO₄), citrate or acetate (CH₃COOH) buffers at a concentration of 100 mM. Leptin (10 μg) was administered orally to wild-type C57BL/6J mice (n=5) in the original vehicle 3 (with 100 mM sodium bicarbonate (NaHCO₃) instead of 125 mM) or in either of these modified versions of vehicle 3. Blood was sampled at time 0, 30, 60 and 120 minutes and processed for plasma leptin levels by EIA determination, as described in Example 7 above.

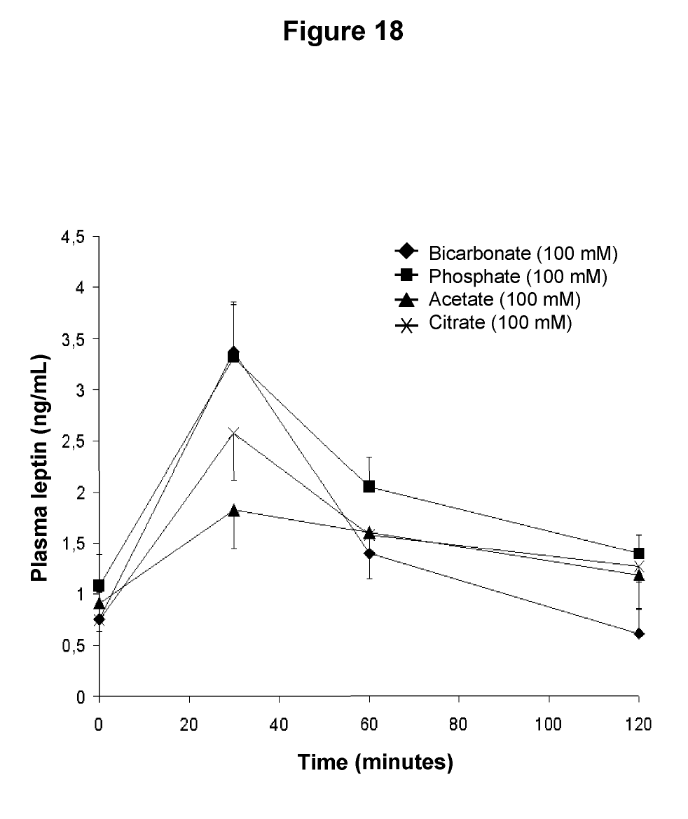

As shown in FIG. 18, phosphate buffer was as efficient as bicarbonate buffer for leptin absorption and seemed to result in a higher sustained plasma leptin levels level over a longer timeframe. Citrate buffer statistically led to lower plasma leptin levels values after 30 minutes. Acetate buffer was found to have the lowest efficiency for plasma leptin levels of the buffers tested. All tested buffers, however, increased plasma leptin levels.

### Example 20

### Effect of pH on Oral Leptin Absorption for Vehicle 3 in Wild-Type C57BL/6J Mice

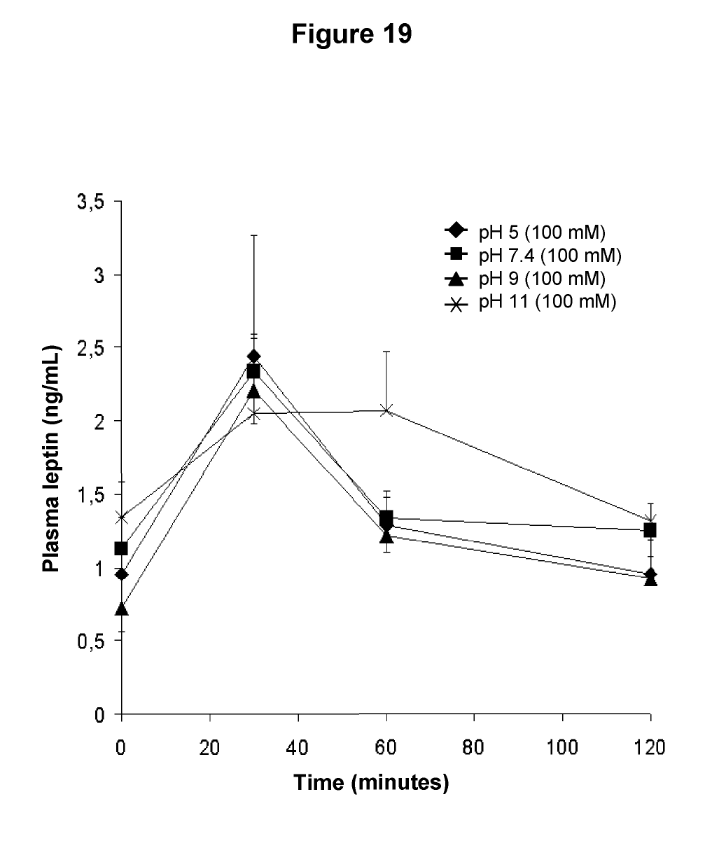

In order to determine the effect of pH of the vehicle on leptin protection/absorption (as suggested by plasma leptin levels), wild-type C57BL/6J mice were forced-fed as described in Example 7 above with leptin (10 μg) in a modified vehicle 3 where a sodium bicarbonate concentration of 100 mM was used and pH values ranging from 5 to 11. Blood was sampled at time 0, 30, 60 and 120 minutes. Plasma leptin levels were determined by EIA as described in Example 2. The results are shown in FIG. 19.

As shown in FIG. 19, over the pH range tested with a bicarbonate buffer concentration of 100 mM. Interestingly, at pH 11, plasma leptin levels remained high at time 60 min, suggesting a better protection and/or absorption of leptin at this pH.

In vitro experiments performed in simulated gastric and duodenal environments

### Example 21

### Effect of pH of the Vehicle on Mouse Leptin Protection in a Simulated Gastric Environment

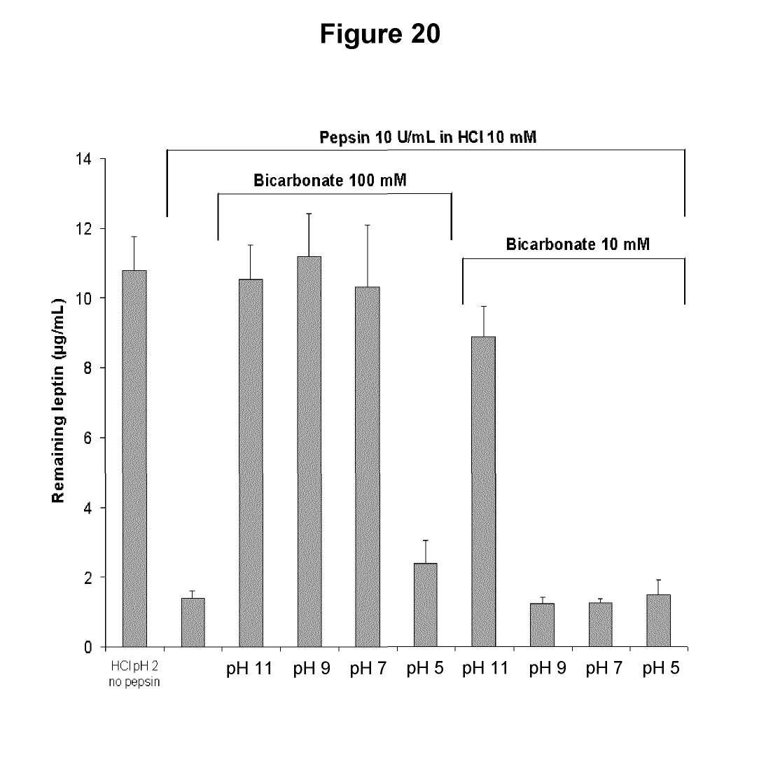

To further characterize the effect of pH of the vehicle on leptin protection/absorption, lower concentrations of bicarbonate in the leptin formulations were used. A vehicle containing 10 mM of sodium bicarbonate was prepared. For greater convenience, these experiments were carried out in vitro. Conditions existing in the stomach were simulated using a solution of HCl (10 mM) containing 10 U/mL of pepsin, the main proteolytic enzyme of the gastric juice. Mouse leptin (10 μg) was incubated for 30 min at 37° C. in the presence of pepsin 10 U/mL in HCl (10 mM) with vehicle 3 modified to contain bicarbonate buffer at 100 mM or 10 mM and at pH values of 5, 7, 9 or 11. Controls consisted of HCl pH 2 (negative control), and pepsin 10 U/mL in HCl pH 2 (positive control). The enzymatic reaction was stopped by adding neutralizing cold sodium bicarbonate buffer (100 mM, v/v) and samples were processed immediately for leptin measurements with the EIA kit (Enzo Life Science, product no. ADI-900-19A), as described in Example 2 above for mouse leptin. The results are shown in FIG. 20 and error bars represent means±SEM (n=5). These results show that pH 11 was able to retain significant amounts of leptin when bicarbonate buffer was present at a concentration of 10 mM.

### Example 22

### Effect of Different Anti-Proteases on the Protection of Human Leptin in a Simulated Gastric Environment

An in vitro system was used to assess the efficiency of different anti-proteases. Human leptin (Cedarlane™, Burlington, Ontario; product No. CLY100-37-5MG; SEQ ID NO: 113) and a corresponding EIA detection kit (R&D Systems, Inc., USA; catalog No. DLP00) that is specific for human leptin were used as described in Example 2 above. Accordingly, the role of anti-proteases in the protection of human leptin in gastric conditions was assessed.

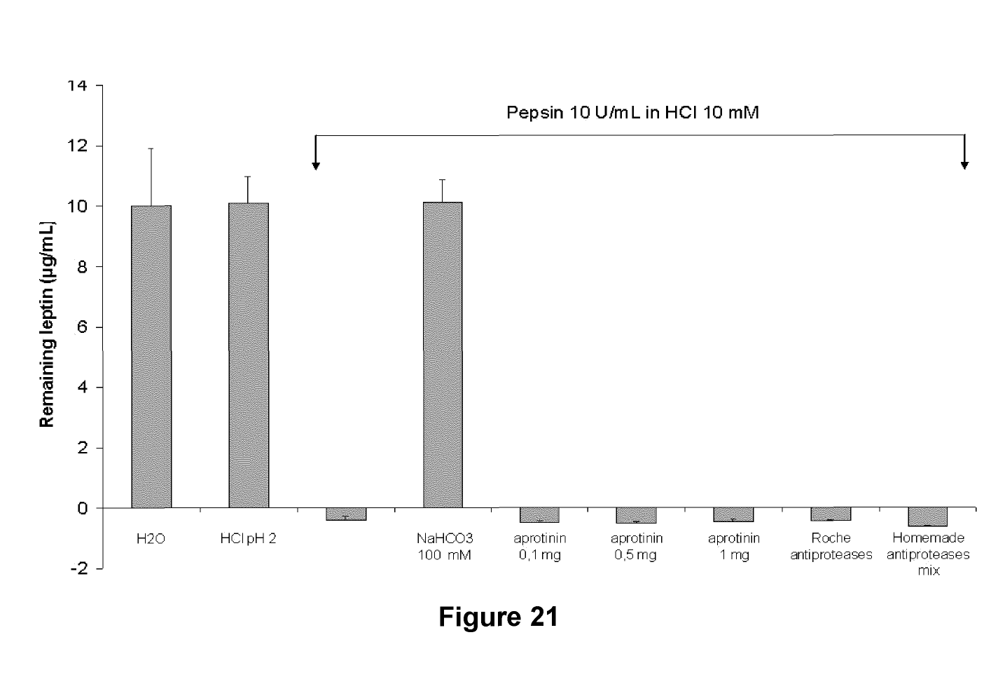

A simulated gastric environment was recreated, as described in Example 21. Human leptin (10 μg) was incubated for 30 min at 37° C. in water (negative control); in HCl (10 mM) (negative control); and in pepsin (10 μg/mL) in HCl 10 mM (positive control). The following components were added to the pepsin 10 μg/mL in HCl 10 mM solution to assess leptin resistance to proteolysis: NaHCO₃ 100 mM (negative control); aprotinin (0.1, 0.5 or 1 mg/mL); commercial anti-protease mix (Roche Diagnostics™; 1 tablet per 10 mL); or an anti-protease mix (aprotinin 10 μg/mL; alpha2-macroglobulin 1 μg/mL; leupeptin 10 μg/mL; chymostatin 10 μg/mL; trypsin inhibitor 10 μg/mL; and PMSF 20 μg/mL). Results are expressed in FIG. 21 as mean±SEM (n=5).

As shown in FIG. 21, leptin was comparably stable in water or HCl pH 2 (see first two bars from the left). In the presence of pepsin in HCl pH 2 (gastric physiologic conditions, see third bar from the left), leptin was completely degraded. Bicarbonate buffer protected the leptin from proteolysis (see fourth bar from the left), most probably by neutralizing the acidic pH required for the optimal enzymatic activity of pepsin. Surprisingly, all of the anti-proteases tested (i.e., aprotinin; commercial anti-protease mix; and our anti-protease mix) were totally inefficient in preventing proteolysis of leptin by pepsin. Interestingly, this was despite the fact that the homemade anti-protease mix contained 1 μg/mL of alpha2-macroglobulin, which has been shown to inhibit pepsin (Athauda et al., 2003).

These results indicate that a stomach acid neutralizing agent (e.g., a buffer) protects leptin in the gastric environment by raising the pH to a level at which pepsin is ineffective.

### Example 23

### Effect of Different Anti-Proteases on the Protection of Human Leptin in a Simulated Duodenal Environment

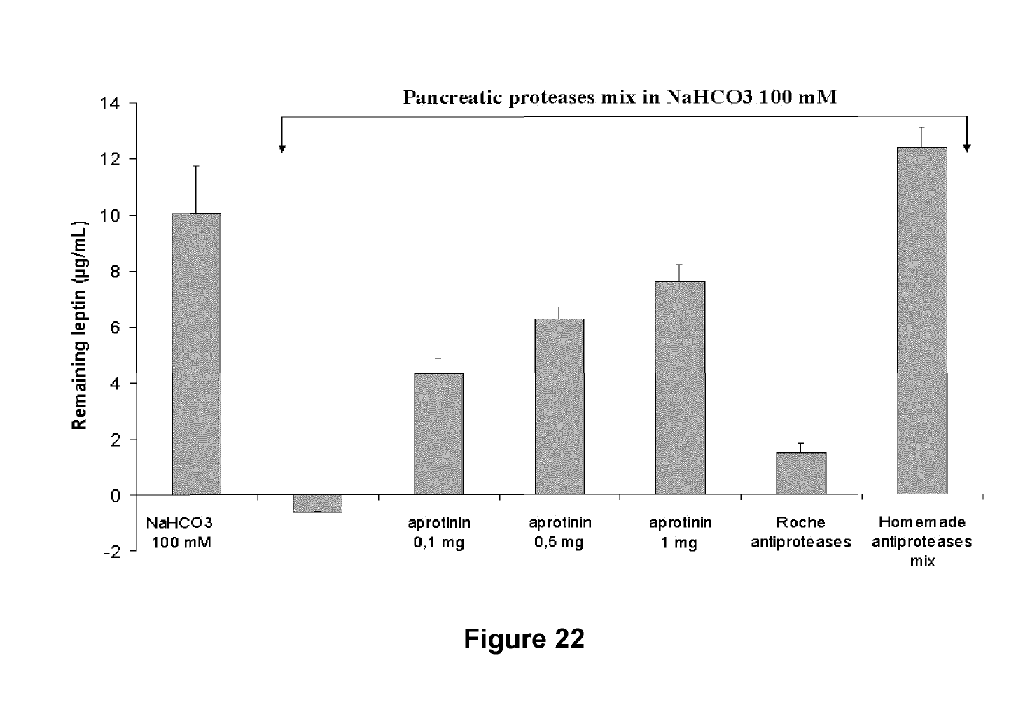

Subsequently, the protective effects of the anti-proteases was assessed on pancreatic enzymes released in the duodenum during digestion. The conditions existing in the duodenal fluid were recreated in vitro to produce a simulated duodenal environment (i.e., NaHCO₃ 50 mM; trypsin 11 U/mL; chymotrypsin 18.4 U/mL; carboxypeptidase 2.5 U/mL and elastase 30 U/mL; hereinafter referred to as “simulated duodenal fluid”). Human leptin (10 μg) was incubated in: NaHCO₃ buffer alone (negative control); the simulated duodenal fluid (positive control) (second bar from the left); and one of the following anti-proteases: aprotinin alone (0.1, 0.5 or 1 mg/mL); commercial anti-protease mix; or an anti-protease mix (aprotinin 10 μg/mL; alpha2-macroglobulin 1 μg/mL; leupeptin 10 μg/mL; chymostatin 10 μg/mL; trypsin inhibitor 10 μg/mL; and PMSF 20 μg/mL). Incubations were carried out for 30 min at 37° C. and the reaction was stopped by adding a mix of anti-proteases at high (saturating) concentration. Samples were tested immediately using a human leptin EIA detection kit (n=5) (R&D Systems, Inc., USA; catalog No. DLP00) as described in Example 2 above. The results are shown in FIG. 22.

As shown in FIG. 22, human leptin was entirely degraded by the mix of pancreatic proteases present in the simulated duodenal environment (see second bar from the left). Aprotinin alone (third to fifth bars from the left) and the homemade mix of anti-proteases (right bar) were quite effective in protecting the human leptin from proteolysis. Surprisingly, the commercial anti-protease mix appeared to be less effective.

These results indicate that anti-proteases/protease inhibitors protect leptin from degradation by pancreatic enzymes (particularly trypsin, the main target of aprotinin) at the level of the duodenum.

### Example 24

### Preparation of Oral Leptin Formulation Using Vehicle 3′

A vehicle 3′ having the following composition was prepared:

Vehicle 3′:

Sodium bicarbonate, 125 mM (or 100 mM, if so indicated)
Sodium deoxycholate, 30 mM
Aprotinin, 1 mg/mL

A 100 mL stock solution of Vehicle 3′ was prepared by dissolving 1.05 g of NaHCO₃, 1.24 g of sodium deoxycholate in 80 mL of distilled water. The mixture was stirred until complete dissolution of all compounds. The pH of the solution was adjusted to 9 using NaOH (10 N and 1N). The volume of the solution was then adjusted to 100 mL and the solution was kept at 4° C.

On the day of an experiment, Aprotinin (1 mg/mL); was dissolved in 10 mL of stock solution (i.e., vehicle 3′). This working solution, in which leptin was dissolved, was kept up to five days at 4° C.

### Example 25

### Effect of Replacing a Commercial Anti-Protease Mix with Aprotinin on Plasma Leptin Levels in Wild-Type C57BL/6J Mice

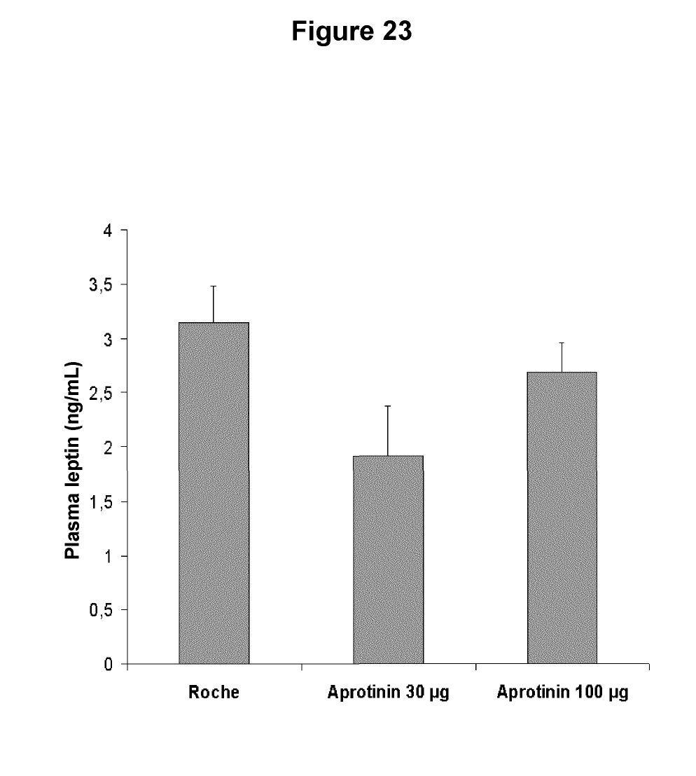

The efficiency of aprotinin alone (i.e., vehicle 3′) instead of the commercial anti-protease mix was then tested in the context of oral leptin administration. Mouse leptin (10 μg) was administered orally to wild-type C57BL/6J mice in vehicle 3 or 3′ containing either a commercial anti-protease mix (from Roche Diagnostics™; 1 tablet/10 mL); or aprotinin alone (30 μg or 100 μg per animal), respectively. Plasma leptin levels were measured 30 minutes after oral administration. Error bars in FIG. 23 represent means±standard deviation (n=5).

As shown in FIG. 23, aprotinin alone (in the absence of the anti-protease mix) protected leptin in a dose-dependent fashion. Aprotinin (100 μg per animal) in vehicle 3′ or the commercial anti-protease mix in vehicle 3 demonstrated similar efficiencies, leading to very similar levels of plasma leptin levels after oral administration of mouse leptin (10 μg) to C57BL/6J mice.

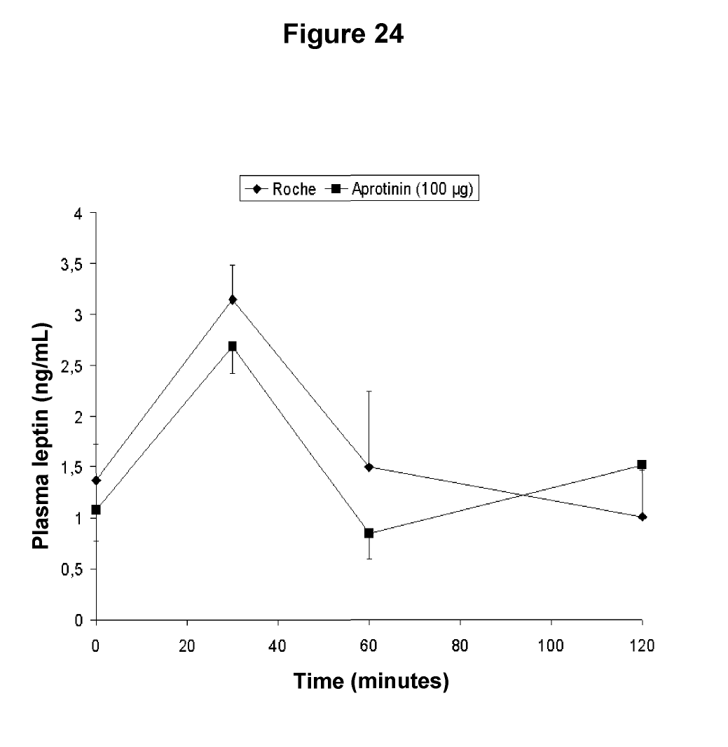

A time-course study was then conducted where plasma leptin measurements were made at time 0, 30, 60 and 120 minutes. Mouse leptin (10 μg) was administered orally to mice in a vehicle containing bicarbonate (100 mM), sodium deoxycholate (30 mM) and either aprotinin (100 μg per animal) or the commercial anti-proteases mix (1 tablet per 10 mL of vehicle 3). As shown in FIG. 24, both vehicles led to a similar rise in plasma leptin levels (n=4) over the measured time points.

These results show that the use of a trypsin inhibitor alone (e.g., aprotinin) can enable effective oral leptin administration.

### Example 26

### Effect of Oral Leptin on Body Weight and Food Consumption of Db/Db Mice

In order to better understand the mechanism by which oral leptin reaches the bloodstream and acts on hypothalamic cells, db/db mice were used. These mice are homozygous for a point mutation in the gene encoding the long isoform of their leptin receptor, which impairs the receptor's activity. These leptin receptors, which are normally expressed in the areas of the hypothalamus involved in the control of food intake, are inactive in db/db mice. Leptin receptor inactivity leads to loss of control of appetite with hyperphagia leading to morbid obesity. The phenotype of the db/db mice is quite similar to that of the ob/ob mice, although their genotypes are different.

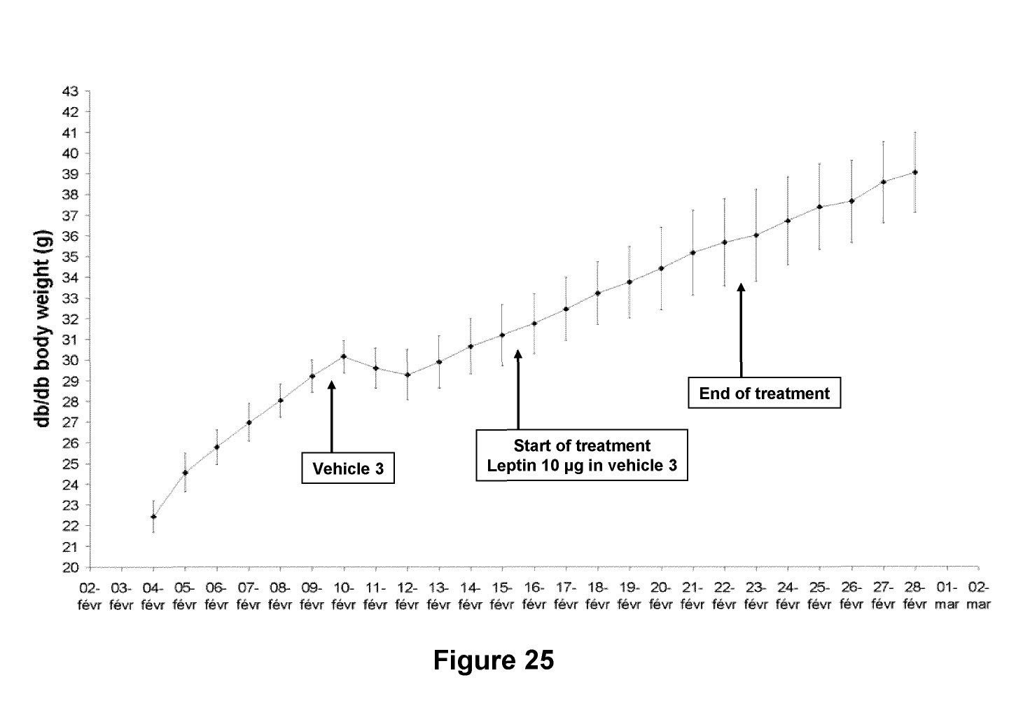

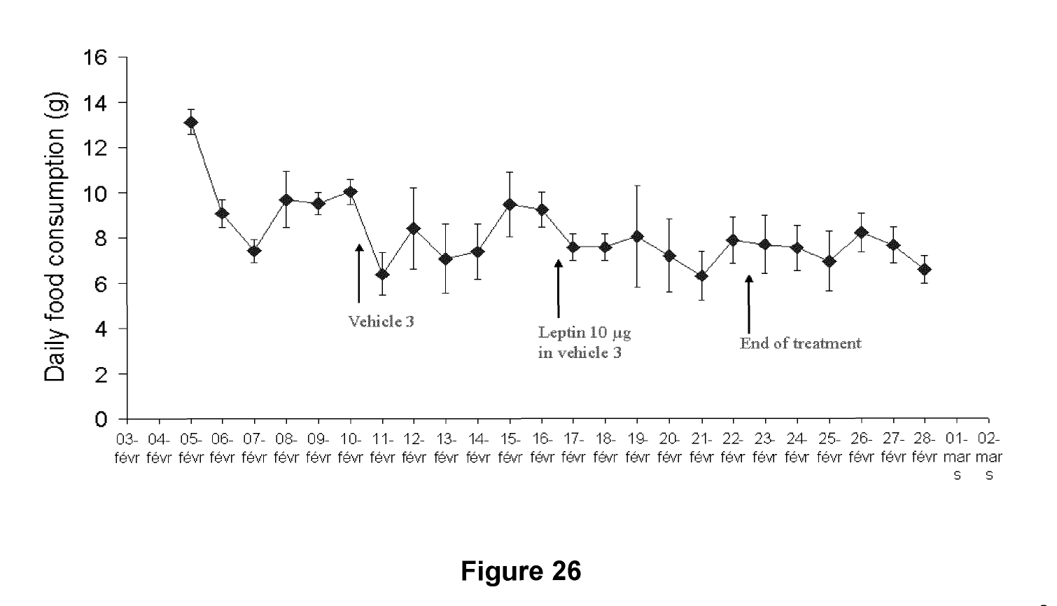

A group of 5 db/db mice was used. Vehicle 3 without leptin was administered for the first 7 days by force feeding using a cannula. As shown in FIG. 25, the force-feeding by itself had an effect and led to an initial small loss of weight, probably because the mice were not yet used to being manipulated. Subsequently, growth was steady and linear. Addition of leptin to vehicle 3 for the 6 following days did not change the rate of body weight gain. Finally, the last 6 days without treatment confirmed that neither the vehicle 3 alone nor oral leptin had an effect on body weight in these animals since they continued gaining weight at the same rate. As shown in FIG. 26, daily food consumption did not show any drastic changes following administration of vehicle or oral leptin. This confirms that the long isoform of the leptin receptor is required to trigger appetite control.

### Example 27

### Effect of Glutamine on the Efficacy of the Oral Leptin Formulation in Wild-Type C57BL/6J Mice

The effect of adding amino acids to the oral leptin formulations of the present invention on plasma leptin levels was then assessed.

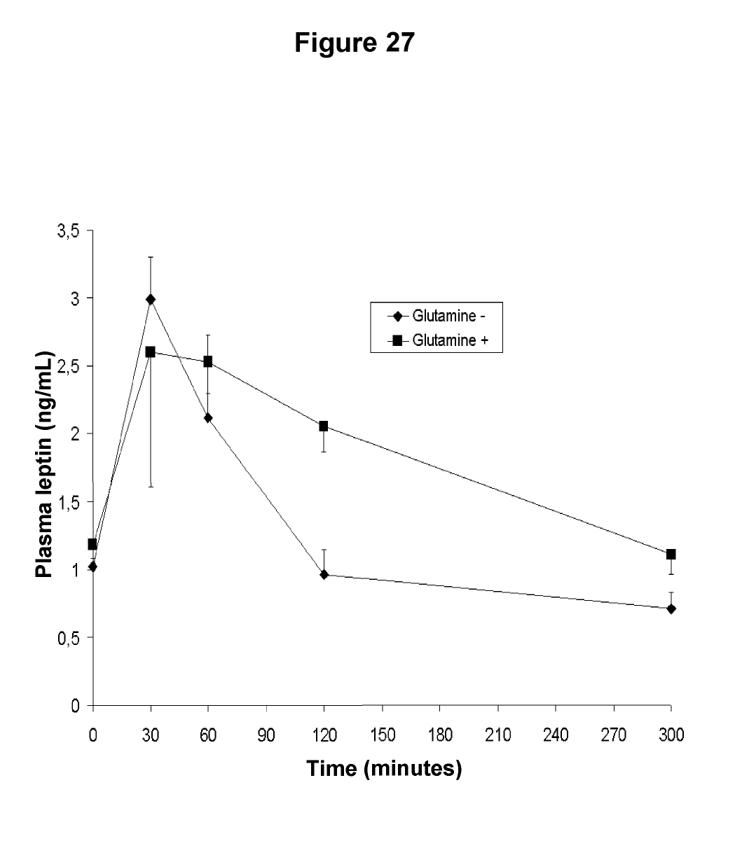

Mouse leptin (10 μg) was formulated in vehicle 3 modified by adding glutamine (“glutamine +”) or original vehicle 3 (i.e., without glutamine (“glutamine −”)) at a concentration of 500 μM, and administered to wild-type C57BL/6J mice by force-feeding. As shown in FIG. 27, in the presence of glutamine, plasma leptin levels remained significantly higher even 2 hours after oral leptin administration. This suggests that glutamine added to the vehicle is able to stimulate endogenous leptin secretion from adipose tissue and to contribute to the overall plasma leptin levels.

Effect of Oral Leptin on Rats

### Example 28

### Effect of Oral Rat Leptin on the Body Weight and Food Intake of Rats

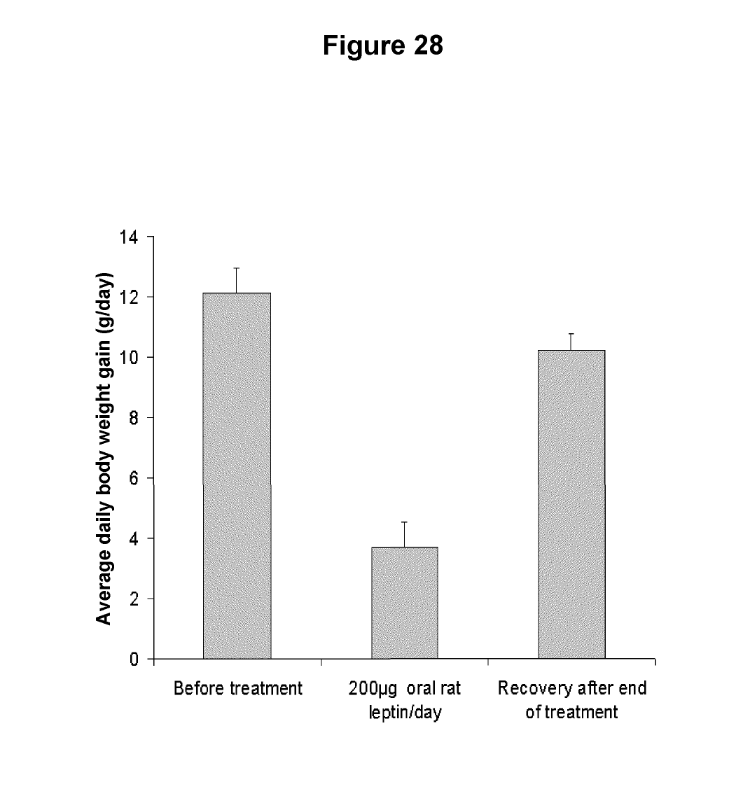

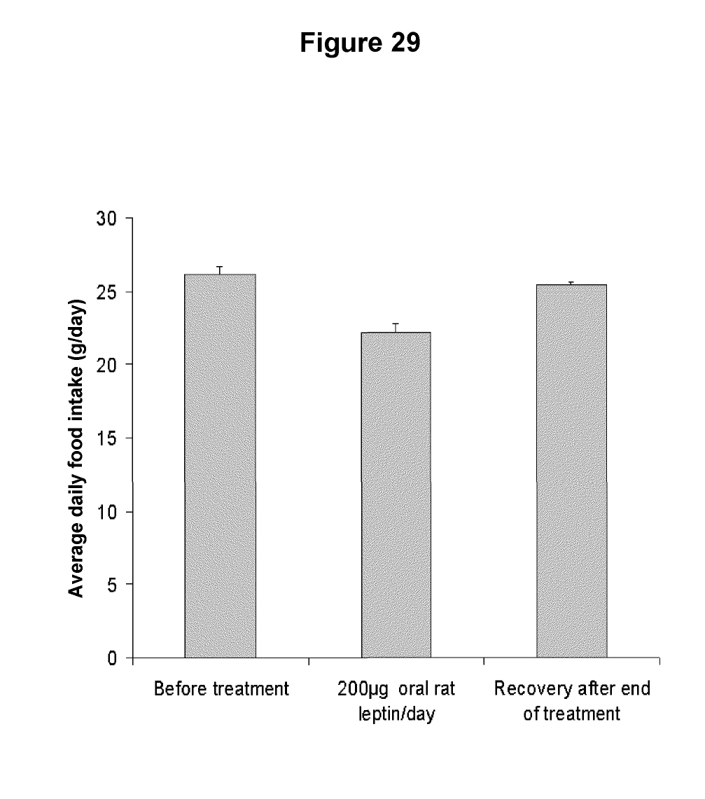

Male Wistar rats (n=5) having a mean body weight of 200 g were monitored for 4 days and then force-fed rat leptin (200 μg) (Cedarlane™, Inc.; Burlington, Ontario; product No. CLY300-14-5MG; SEQ ID NO: 114) using a cannula. formulated in vehicle 3′ for 4 days. The rats were then followed for a recovery period of 4 days. Body weight and food intake were measured daily over the entire period. The results are shown in FIGS. 28 and 29, in which: the left-most bar represents mean values of 4 days of observation without any treatment; the middle bar represents mean values after daily forced-feeding of rat leptin (200 μg) in vehicle 3 for 4 days; and the right most bar represents animals which were allowed to recover for another 4 days after the end of leptin treatment.

FIG. 28 shows that oral leptin treatment was very efficient in reducing the average daily body weight gain (i.e., the rate of weight gain) of the rats. That is, the rats did not lose weight as was also observed for ob/ob and C57BL/6J mice. Rather, the rats continued gaining weight but their daily increase in body weight was reduced by 70% (from an average of 11.66±1.08 g per day without treatment, to 3.71±0.79 g per day with oral leptin). The growth of the rats administered oral leptin was significantly slowed down.

Interestingly, FIG. 29 shows that the average daily food intake was reduced by the leptin treatment (from 26.1±0.52 g per day without treatment to 22.16±0.62 g per day with oral leptin).

Like previously observed in mice, the body weight changes and food intake of the rats were restored to normal levels, shortly after the oral leptin treatment was stopped.

### Example 29

### Effect of Oral Rat Leptin on Plasma Leptin Levels in Rats

Rat leptin (150 μg) (Cedarlane™, Inc.; Burlington, Ontario; product No. CLY300-14-5MG; SEQ ID NO: 114) was dissolved in a vehicle 3′ as described in Example 24 above, i.e., containing: bicarbonate (100 mM); sodium deoxycholate (30 mM); and aprotinin (1 mg). Rats (n=5) were force-fed and blood was sampled at time 0, 30, 60, 120 and 300 minutes. Samples were analyzed using a rat leptin EIA kit (R&D Systems, Inc., USA; catalog No. MOB00) as described in Example 2.

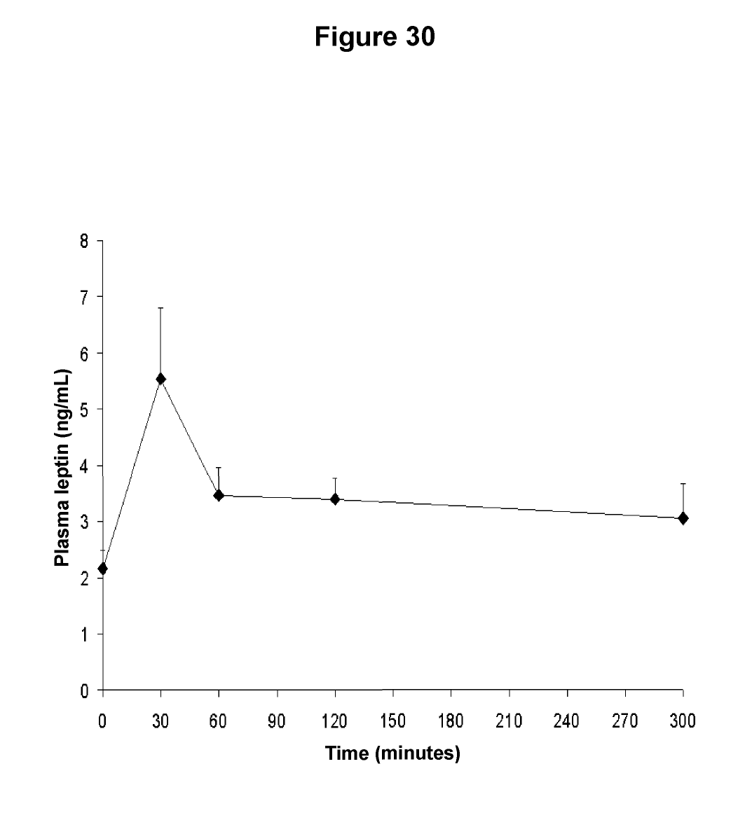

As shown in FIG. 30, plasma leptin levels rise in a similar fashion as previously observed for C57BL/6J or ob/ob mice.

### Example 30

### Effect of Oral Human Leptin on Daily Body Weight Change, Daily Food Intake and Plasma Leptin Levels in Rats

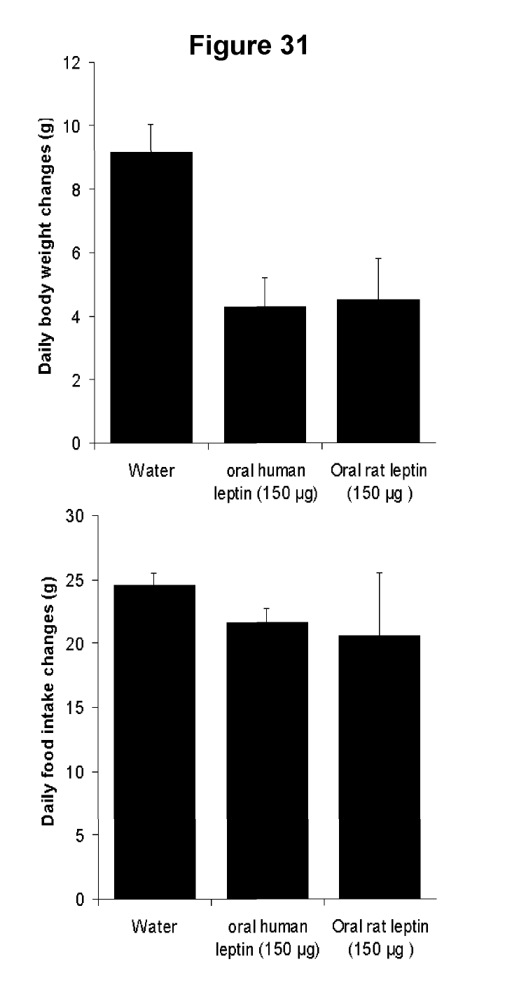

To confirm that the vehicle protects human leptin while keeping its biological activity, Wistar rats were orally administered rat or human leptin (150 μg) in vehicle 3′ for 2 days. Body weight and food intake were measured and compared to a control group force-fed with water. Results in FIG. 31 are expressed as means of two days of treatment (n=3 animals). As shown in FIG. 31, rat and human leptins reduce rat weight gain and food intake to substantially the same extent.

Administration of Oral Leptin with Food Compared to without Food

### Example 31

### Effect of Mouse Leptin Administered Orally to Wistar Rats with Food

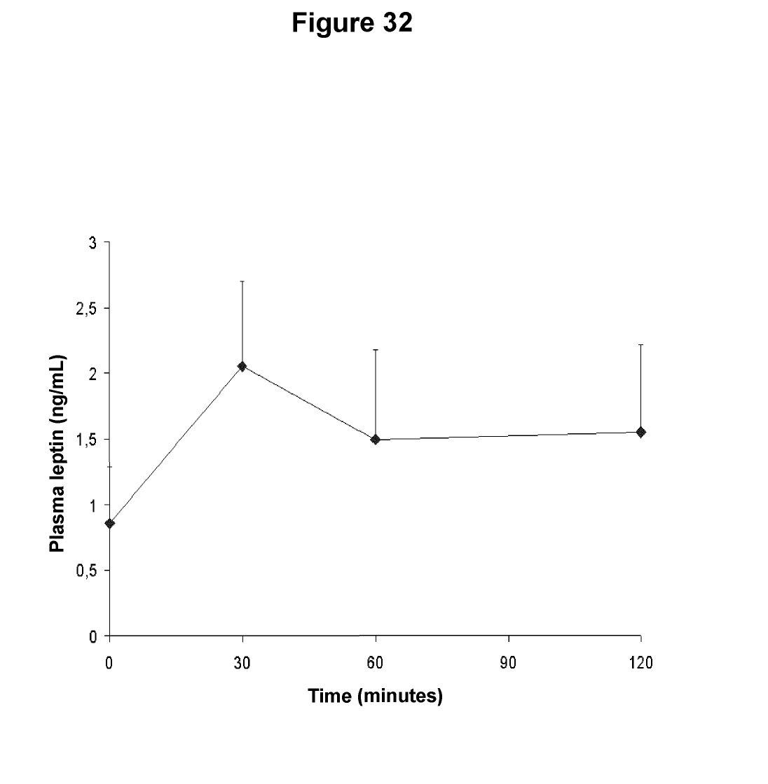

Mouse leptin (100 μg) was dissolved in vehicle 3′ and standard Purina Chow™ was then soaked with this mouse leptin in vehicle 3′. Wistar rats (n=5) were fasted for 18 hours before they were administered the leptin soaked chow to ensure low leptin levels at time 0 and to be sure that the animals would be hungry and eat the leptin soaked chow entirely. Blood samples were taken prior to feeding and then at time 30, 60 and 120. Blood samples were analyzed with EIA leptin kit (Enzo Life Science; product no. ADI-900-19A) specific for the exogenously administered mouse leptin (n=5) as described in Example 2. The rats started to eat the soaked chow as soon as it was given. Time 0 was chosen when half of the chow was eaten. The results shown in FIG. 32 confirmed that rats indeed absorb the exogenously administered mouse leptin when present in food, and that it is delivered to the bloodstream.

### Example 32

### Effect of Human Leptin Administered Orally to Wistar Rats with Food

In order to assess the specificity of leptin absorption, a similar experiment was carried out with standard Purina Chow™ soaked with a vehicle solution containing human leptin.

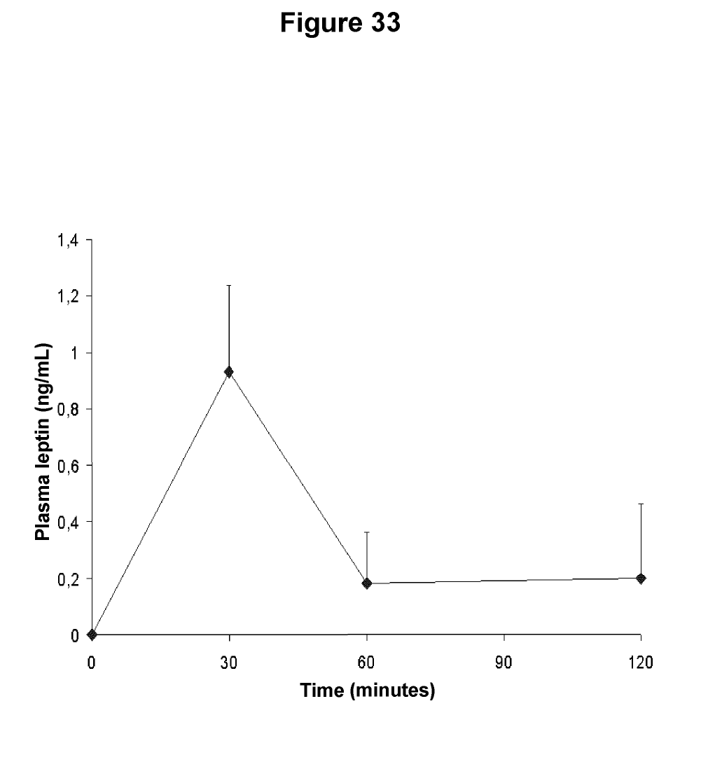

Human leptin (100 μg) (Cedarlane™, Burlington, Ontario; product No. CLY100-37-5MG; SEQ ID NO: 113) was dissolved in vehicle 3′. Standard Purina™ chow was then soaked with this human leptin formulation and given to 18 hrs-fasted Wistar rats. Blood samples were sampled prior to feeding and then at time 30, 60 and 120. Blood samples were analyzed with EIA human leptin kit (R&D Systems, Inc., USA; catalog No. DLP00) specific for the exogenously administered human leptin (n=5) as described in Example 2. The EIA kit for human leptin is very specific and detects only human leptin. The results shown in FIG. 33 confirmed that rats indeed absorb the exogenously administered human leptin when present in food, and that the exogenously administered human leptin is delivered to the bloodstream.

### Example 33

### Comparison of the Absorption of Rat Leptin Administered Orally with or without Food

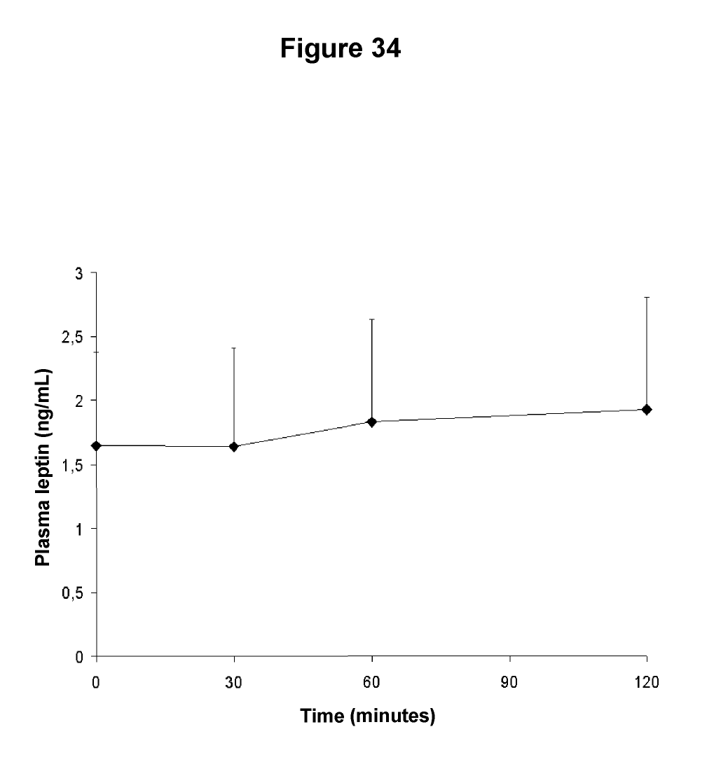

As intake of Purina Chow™ may have triggered by itself an increase in endogenous plasma leptin levels, 18 hours-fasted Wistar rats standard were given 2 g of Purina Chow™ devoid of leptin (but soaked in vehicle 3′) and blood was sampled at time 30, 60 and 120 minutes. Blood samples were immediately processed for leptin measurements using a rat leptin EIA kit (n=5) (R&D Systems, Inc., USA; catalog No. MOB00) as described in Example 2. The results of this control experiment are shown in FIG. 34.

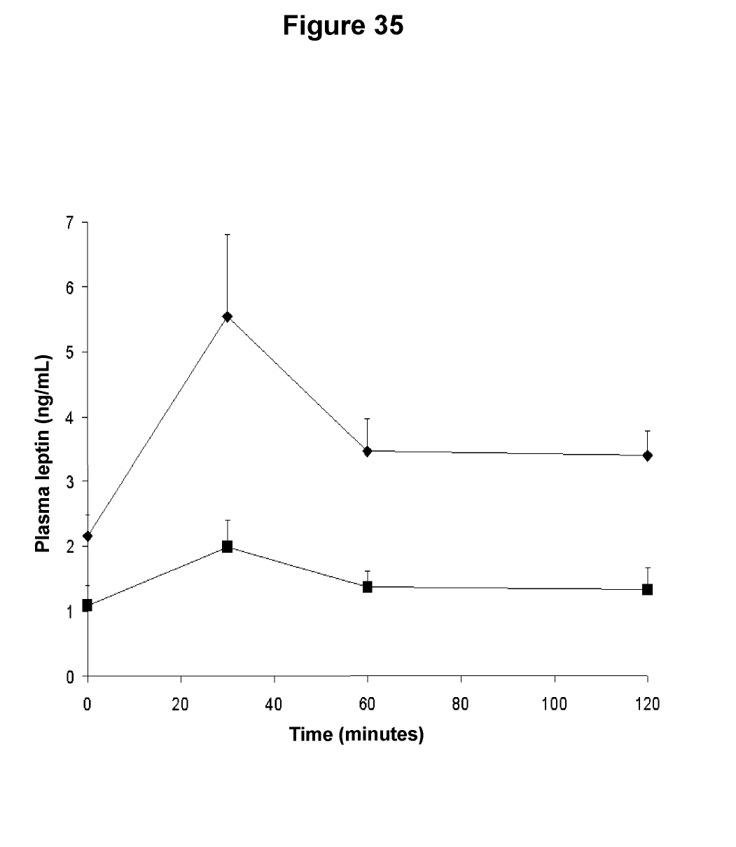

Subsequently, substantially the same protocol as in Examples 30 and 31, was carried out except using Purina Chow™ soaked in rat leptin (150 μg) and results were compared with the oral administration of rat leptin (150 μg) without food (i.e., by force-feeding). Blood samples were immediately processed for leptin measurements using a rat leptin EIA kit (n=5) (R&D Systems, Inc., USA; catalog No. MOB00) as described in Example 2 above. The results are shown in Table III below and plotted in FIG. 35 (leptin without food, diamonds; leptin with food, squares).

TABLE III

| | Plasma leptin levels (ng/mL) | |
|---|---|---|
| | Forced-fed oral | Purina soaked in |
| Time | rat leptin (150 μg) | rat leptin (150 μg) |

| (min) | Mean | Stdev | Mean | Stdev |
|---|---|---|---|---|
| 0 | 2.156 | 0.329 | 1.096 | 0.308 |
| 30 | 5.538 | 1.265 | 1.986 | 0.419 |
| 60 | 3.462 | 0.510 | 1.367 | 0.245 |
| 120 | 3.399 | 0.373 | 1.327 | 0.344 |

As can be seen from the values in Table III, as well as FIG. 35, forced-feeding and leptin soaked Purina™ led respectively to a 257% (from 2.156 to 5.538 ng/mL) and 182% (from 1.096 to 1.986 ng/mL) increase in plasma leptin levels from 0 to 30 minutes (n=5). In comparison, there was no significant increase in endogenous plasma leptin levels for this same time interval in the control experiment in which 2 g of food was given (FIG. 34). Both administration methods (i.e., with and without food) led to significant increases of plasma leptin levels.

### Example 34

### Comparison Between Oral and Intraperitoneal Administration of Leptin

Wild-type C57BL/6J mice (n=7) were intraperitoneally (IP) injected with saline solution (“IP saline”)(150 μL) for three days. A dose of 2.5 μg of mouse leptin, shown above (when given orally) to reduce body weight and food intake without having a maximal effect was used. This represents an optimal condition for this type of study.

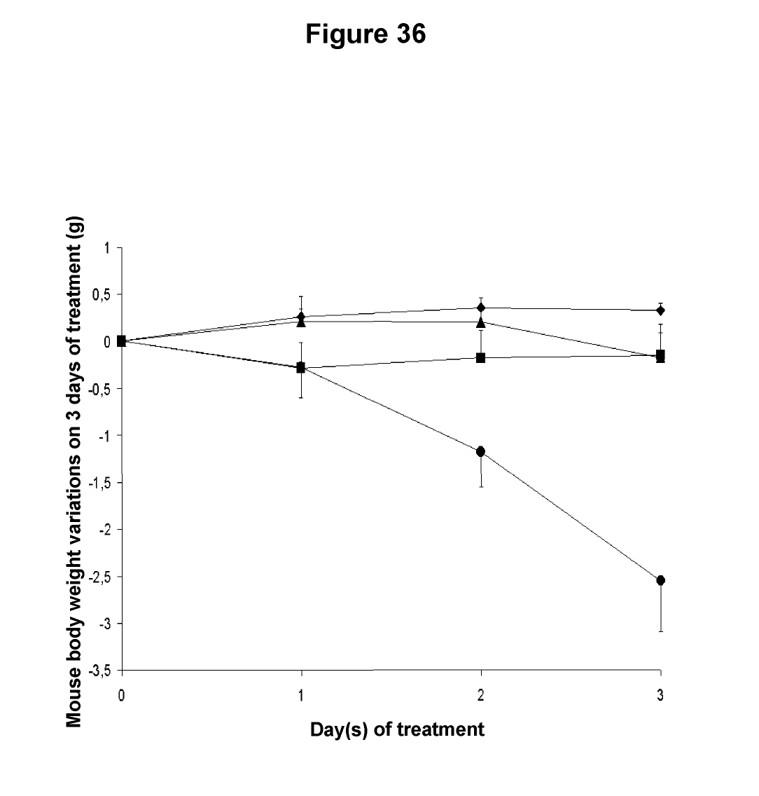

After a two-day recovery, the mice received a daily IP injection of mouse leptin (2.5 μg)(“IP leptin”) in saline (150 μL) for another three days. After two days of recovery, they were orally forced-fed with vehicle 3′ (150 μL) (“oral vehicle”) for three days. Two days later, they received an oral administration of mouse leptin (2.5 μg) in vehicle 3′ (150 μL) (oral leptin). Mice body weight was measured daily and the results are shown in FIG. 36, Diamonds correspond to weight variation over the three days after IP saline injection; squares correspond to weight variation over the three days after IP leptin injection; triangles correspond to weight variation over the three days after oral vehicle force feeding; and circles correspond to weight variation over the three days after oral leptin force feeding. Body weight variations are presented using day 0 as reference (n=7).

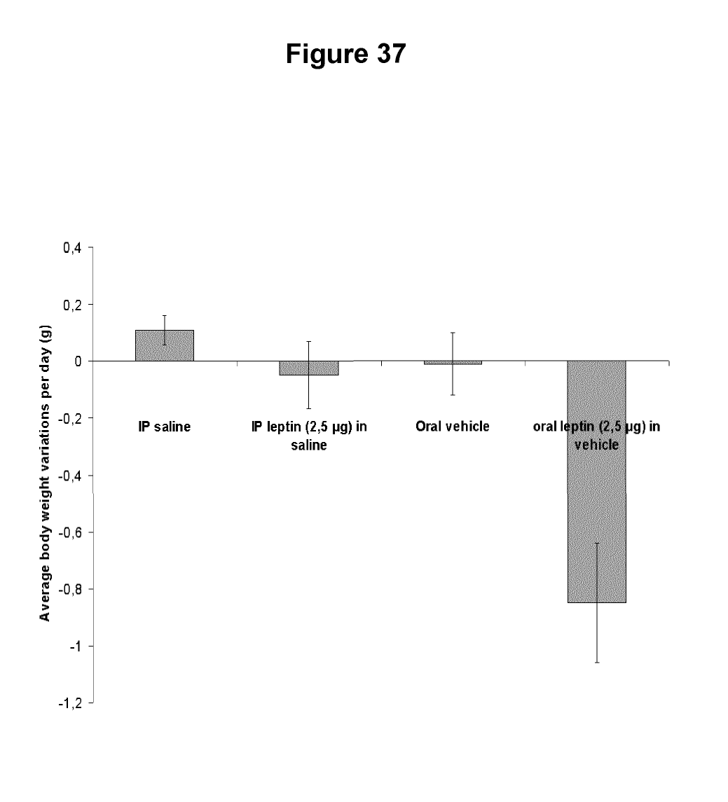

FIG. 37 shows the average daily body weight changes in the mice. Error bars represent the mean SEM of mean values of body weight daily variations over three days (n=7).

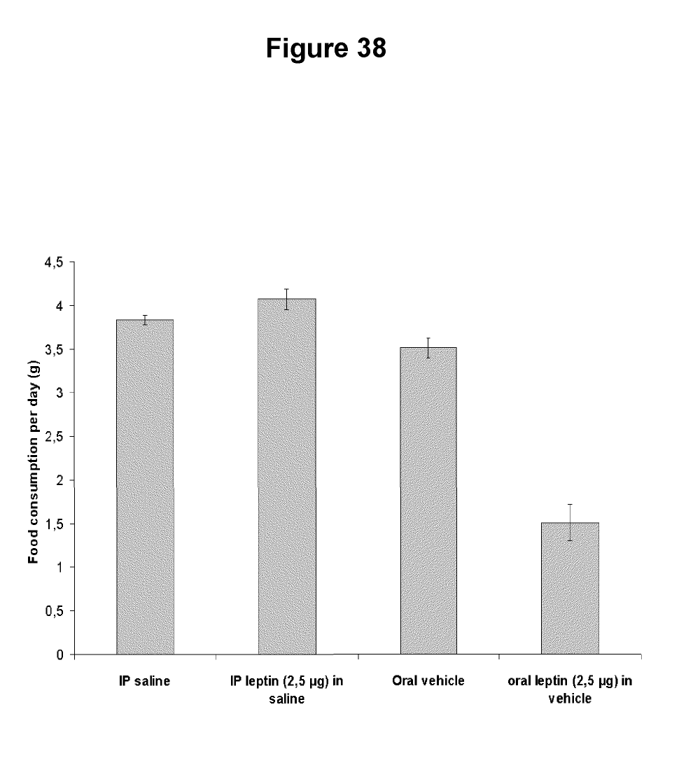

FIG. 38 shows the average food consumption during intraperitoneal leptin or oral leptin treatment of the C57BL/6J mice. Food consumption was measured daily. Error bars represent the mean±SEM of food eaten per day over three days (n=7).

The results in FIGS. 36-38 collectively show that oral administration of leptin is far more efficient than IP injection of the same amount of leptin for reducing body weight and for decreasing food consumption.

### Example 35

### Histo-Pathological Examination of Mice Administered Oral Leptin

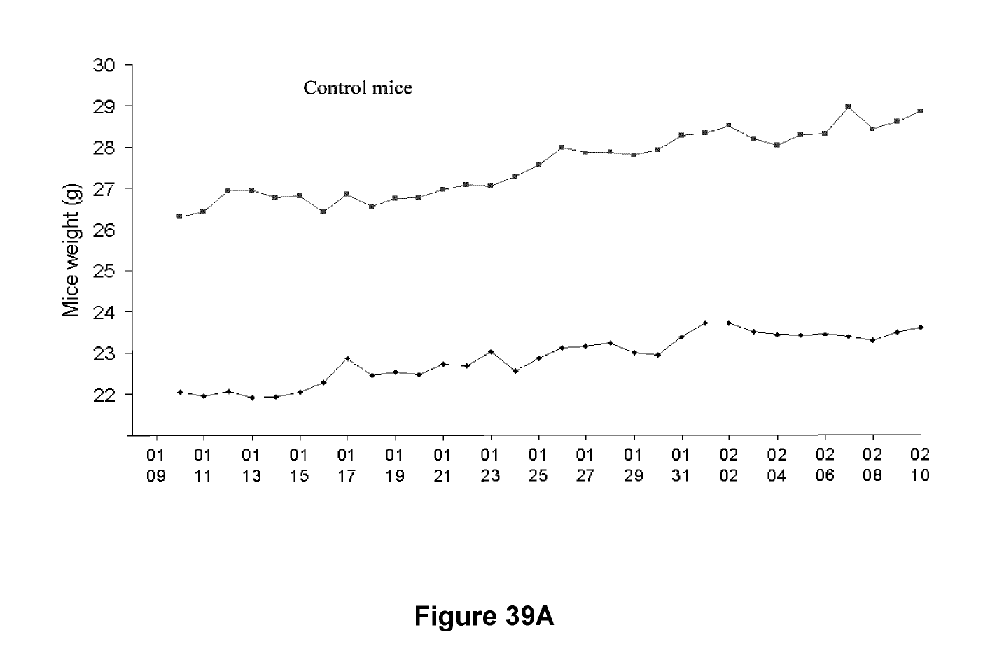

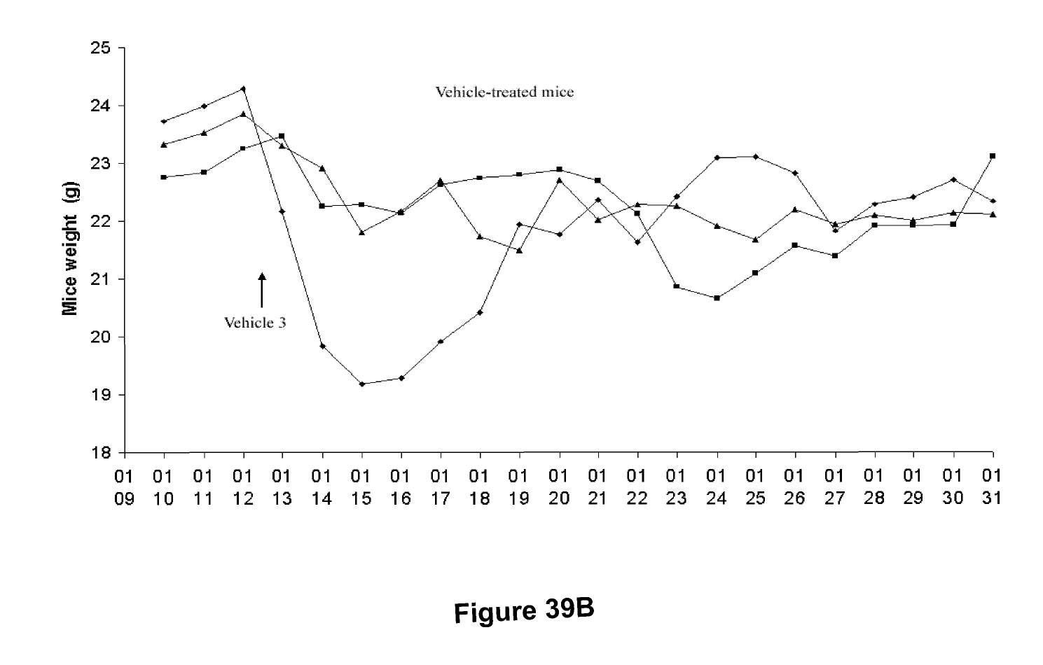

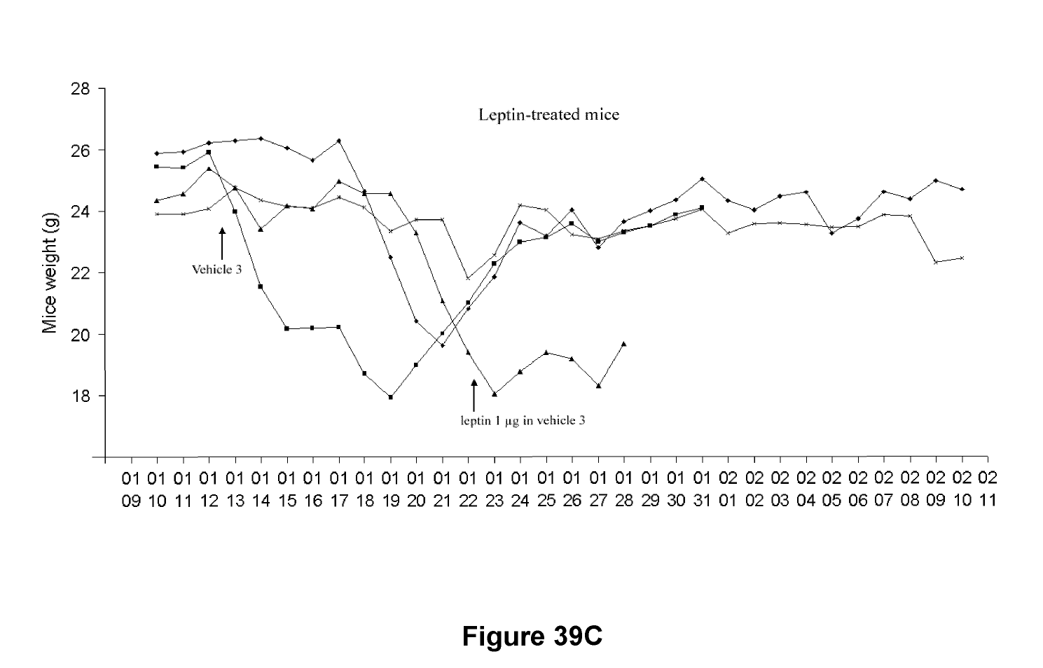

In order evaluate any potential histo-pathological effects of the oral leptin formulations of the present invention, wild-type C57BL/6J mice were divided into three groups: one group received nothing (“Control mice”, FIG. 39A)(n=2); one group received the vehicle 3 alone by force-feeding (“Vehicle-treated mice”, FIG. 39B) (n=3); and one group received 1 μg of leptin a day beginning at the indicated day in vehicle 3 by force-feeding (“Leptin-treated mice”, FIG. 39C) (n=4).

In order to observe mice for a long period of time, 1 μg of leptin was used to stabilize the weight of C57BL/6J mice. Mice from each group were weighted daily, and results are shown in FIGS. 39A, 39B, and 39C (“Control mice”, “Vehicle-treated mice”, “leptin-treated mice”, respectively), each line corresponding to individual mice.

For the “Vehicle-treated mice” (FIG. 39B), vehicle 3 was administered on the third day after beginning daily weight measures (i.e., January 12^{th}), as indicated by the arrow. For the “leptin-treated mice” (FIG. 39C) vehicle 3 was administered on the third day after beginning daily weight measures (i.e., January 12^{th}) and the leptin in vehicle 3 was administered on the thirteen day after beginning daily weight measures (i.e., January 22^{th}), as indicated by the arrows.

The mice were kept for up to 30 days before being sacrificed. Tissues (stomach, intestine and liver) were sampled after 10, 20 and 30 days of treatment under anesthesia. The tissues were then fixed in Bouin buffer, prepared for microscopy according to standard histo-pathological procedures, and examined using a light microscope and a transmission electron microscope. In addition, the tissues were examined by a trained clinical pathologist and an official histo-pathological report was prepared. The official report confirmed the inventors observations, and demonstrates that examined tissues show little to no alterations as a result of the oral leptin administration. The histo-pathological report from the histo-pathologist is shown below.

TABLE IV
Histopathological assessment of mice tissues.

| | ANIMAL ID | | | | | | | |
|---|---|---|---|---|---|---|---|---|
| | 5542 | 5544 | 5545 | 5546 | 5548 | 5549 | 5559 | 5560 |
| | V | V | V | L | C | L | L | C |
| Stomach | | | | | | | | |
| Forestomach | | | | | | | | |
| Hyperplasia/ | 2 | 0 | 0 | 0 | 0 | 1 | 0 | 0 |
| Keratosis | | | | | | | | |
| Ulceration | 0 | 0 | 0 | 0 | 0 | 0 | 0 | 0 |
| Inflammation | 1 | 0 | 0 | 0 | 0 | 0 | 0 | 1 |
| Glandular | | | | | | | | |
| Stomach | | | | | | | | |
| Hyperplasia | 0 | 0 | 0 | 0 | 0 | 0 | 0 | 0 |
| Ulceration | 0 | 0 | 0 | 0 | 0 | 0 | 0 | 0 |
| Inflammation | 0 | 0 | 0 | 0 | 0 | 0 | 0 | 1 |
| Intestine | | | | | | | | |
| Ratio villi/crypt | 8.0 | 5.3 | 6.2 | 2.6 | 5.91 | 5.93 | 8.1 | 6.0 |
| Villi density | 0 | 0 | 0 | 1 | 1 | 1 | 1 | 0 |
| reduction | | | | | | | | |
| Blunted villi | 0 | 0 | 0 | 1 | 0 | 1 | 0 | 0 |
| Crypt | 0 | 0 | 0 | 2 | 0 | 0 | 0 | 0 |
| hyperplasia | | | | | | | | |
| Ulceration | 0 | 0 | 0 | 0 | 0 | 0 | 0 | 0 |
| Inflammation | 0 | 0 | 0 | 0 | 0 | 0 | 0 | 0 |
| Liver | | | | | | | | |
| Steatosis | 0 | 0 | 0 | 0 | 0 | 0 | 0 | 0 |
| Bile retention | 0 | 0 | 0 | 0 | 0 | 0 | 0 | 0 |
| Fibrosis | 0 | 0 | 0 | 0 | 0 | 0 | 0 | 0 |
| Hepatocellular | 0 | 0 | 0 | 0 | 0 | 0 | 0 | 0 |
| necrosis | | | | | | | | |
| Portal | 0 | 0 | 0 | 0 | 0 | 0 | 0 | 0 |
| inflammation | | | | | | | | |
| Micro- | 0 | 1 | 0 | 1 | 0 | 0 | 1 | 2 |
| granulomas | | | | | | | | |
| Kupfer cell | 0 | 0 | 1 | 0 | 0 | 0 | 1 | 0 |
| hypeplasia | | | | | | | | |

Scoring Grid
0 = Absent
1 = Minimal
2 = Moderate
3 = Severe
C: control animals
V: vehicle-treated animals
L: leptin-treated animals

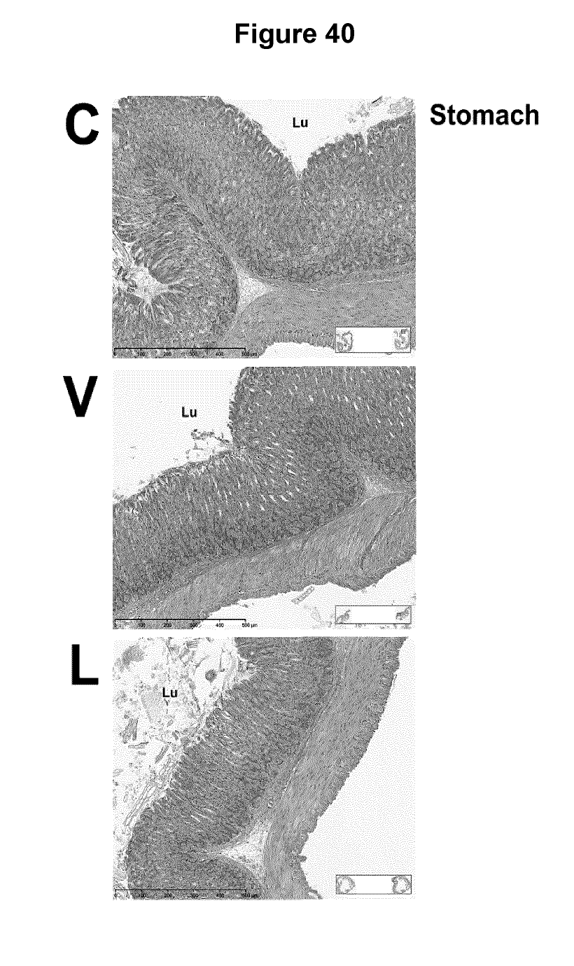

Exemplary images of the stomach, duodenum, and liver tissues that were examined are shown in FIGS. 40-45. More particularly:

FIG. 40 shows images taken of stomach tissue via light microscopy of the gastric wall in Controls, Vehicle-treated and Leptin-treated C57BL/6J mice (panels “C”, “V”, and “L”, respectively). “Lu” represents the gastric lumen.

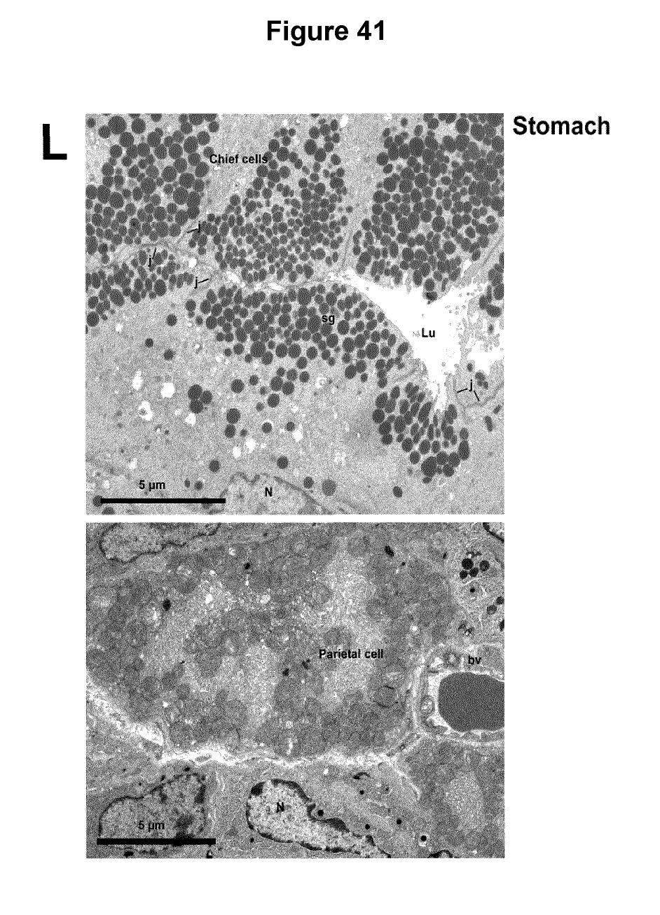

FIG. 41 shows images taken of stomach tissue via electron microscopy of the gastric mucosa of a leptin-treated C57BL/6J mouse (“L”, referring to both upper and lower panels). “Lu” represents gastric lumen; “N” represents nucleus; “bv” represents blood vessels; “sg” represents secretory granules; “j” represents intercellular junctions.

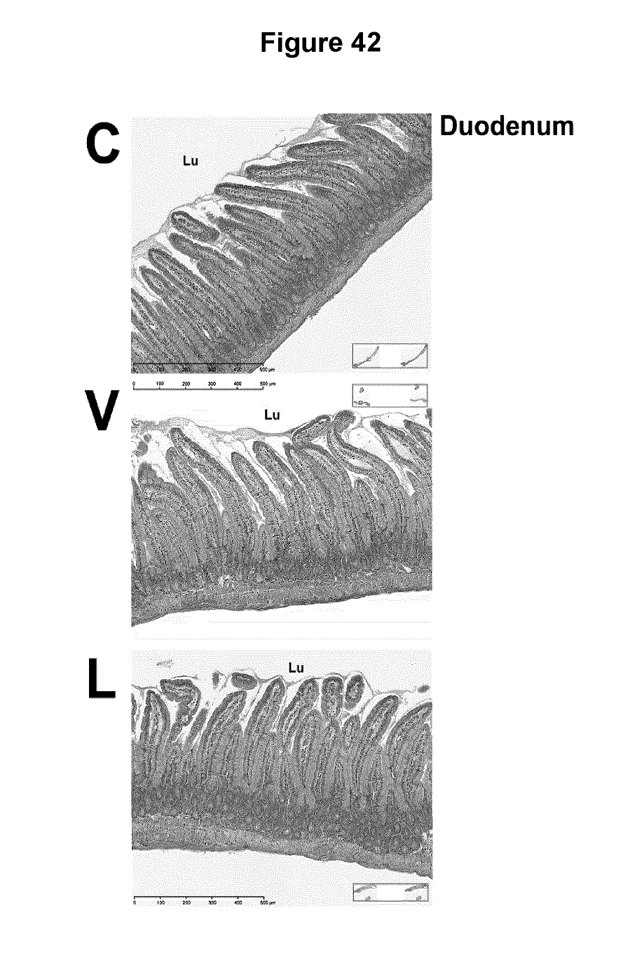

FIG. 42 shows images taken of duodenum tissue via light microscopy in Controls, Vehicle-treated and Leptin-treated C57BL/6J mice (panels “C”, “V”, and “L”, respectively). “Lu” represents the gastric lumen.

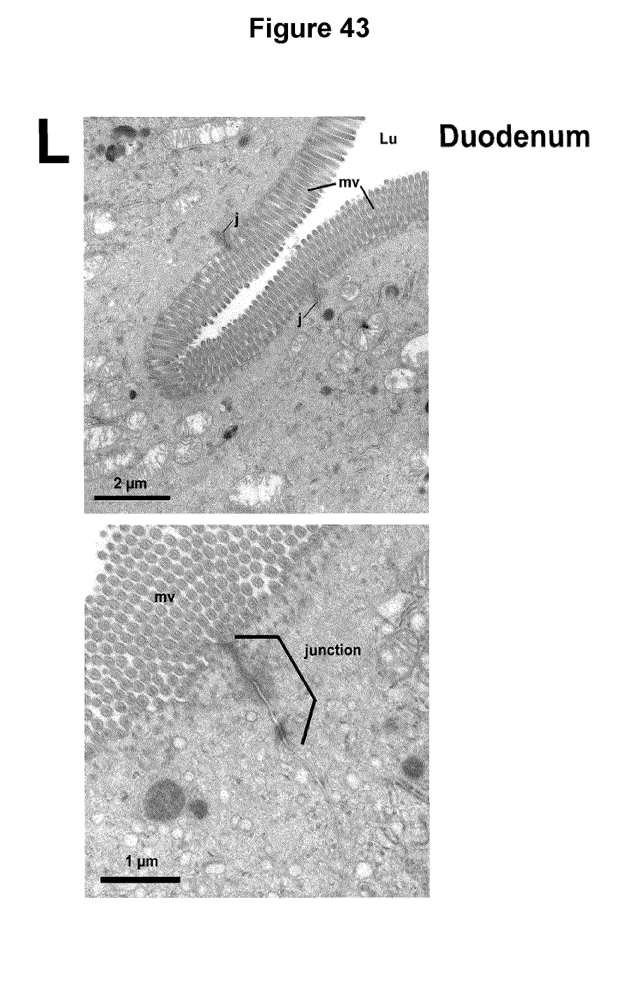

FIG. 43 shows images taken of duodenum tissue via electron microscopy of the duodenal mucosa of a leptin-treated C57BL/6J mouse (“L”, referring to both upper and lower panels). “Mv” represents microvilli; “j” represents intercellular junctions; “Lu” represents duodenal lumen.

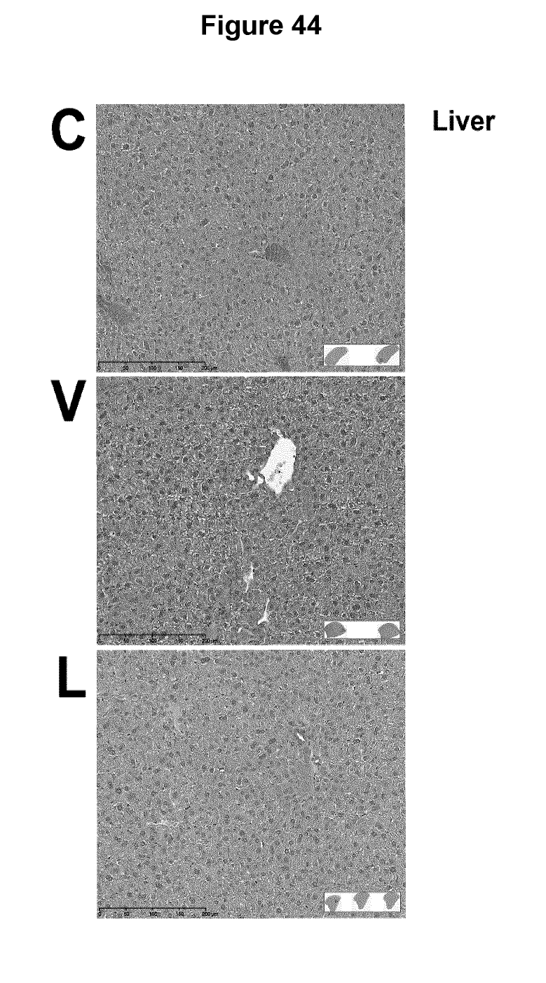

FIG. 44 shows images taken of liver tissue via light microscopy in Controls, Vehicle-treated and Leptin-treated C57BL/6J mice (panels “C”, “V”, and “L”, respectively).

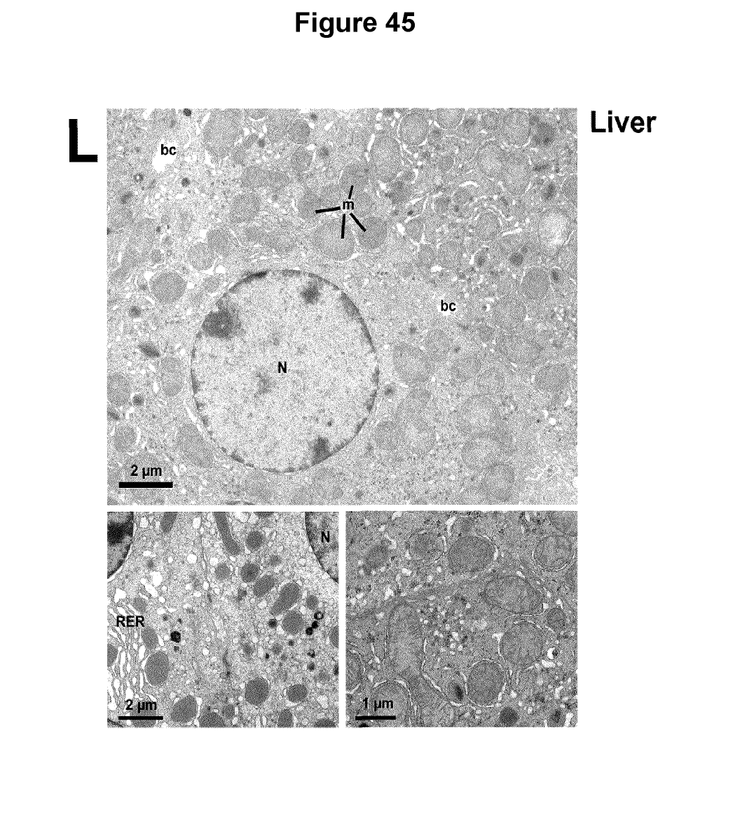

FIG. 45 shows images taken of liver tissue via electron microscopy from a leptin-treated C57BL/6J mouse (“L”, referring to both upper and lower panels). “N” represents nucleus; “m” represents mitochondria; “bc” represents bile canaliculi; “RER” represents rough endoplasmic reticulum.

### Example 36

### Stabilization of Body Weight of ob/ob Mice by Daily Administration of Oral Leptin

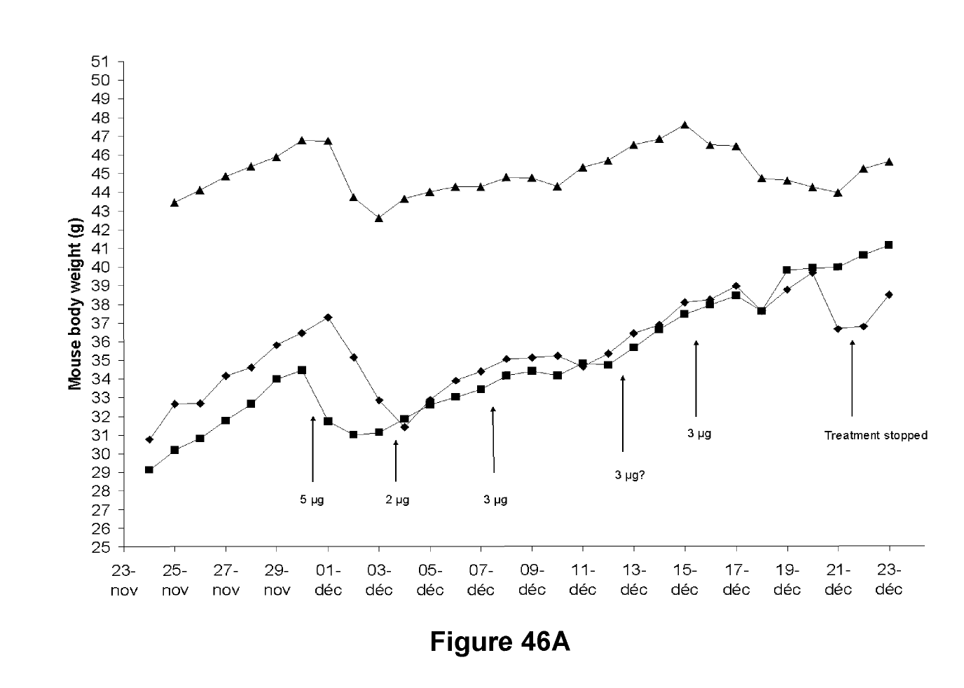

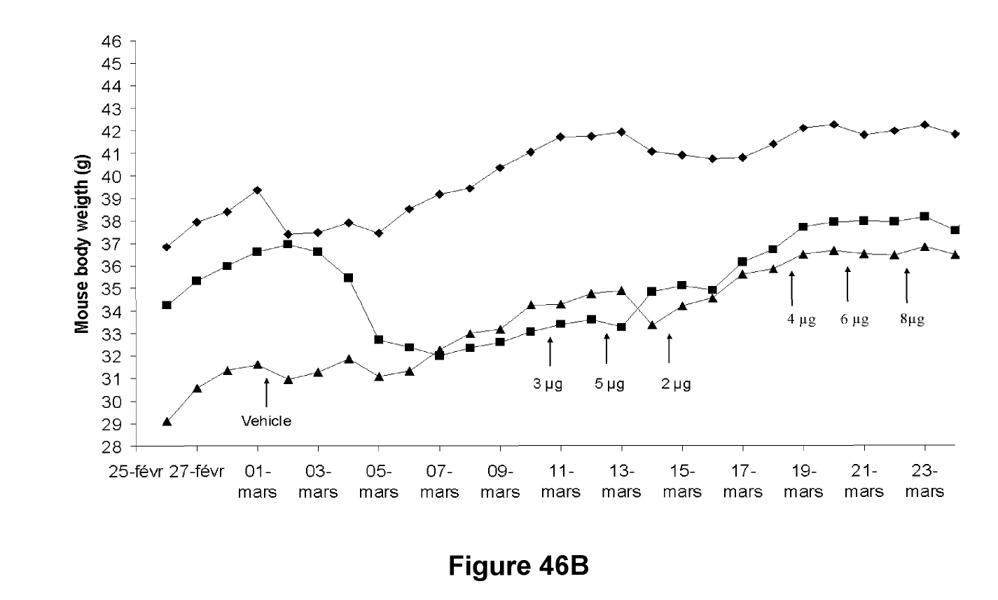

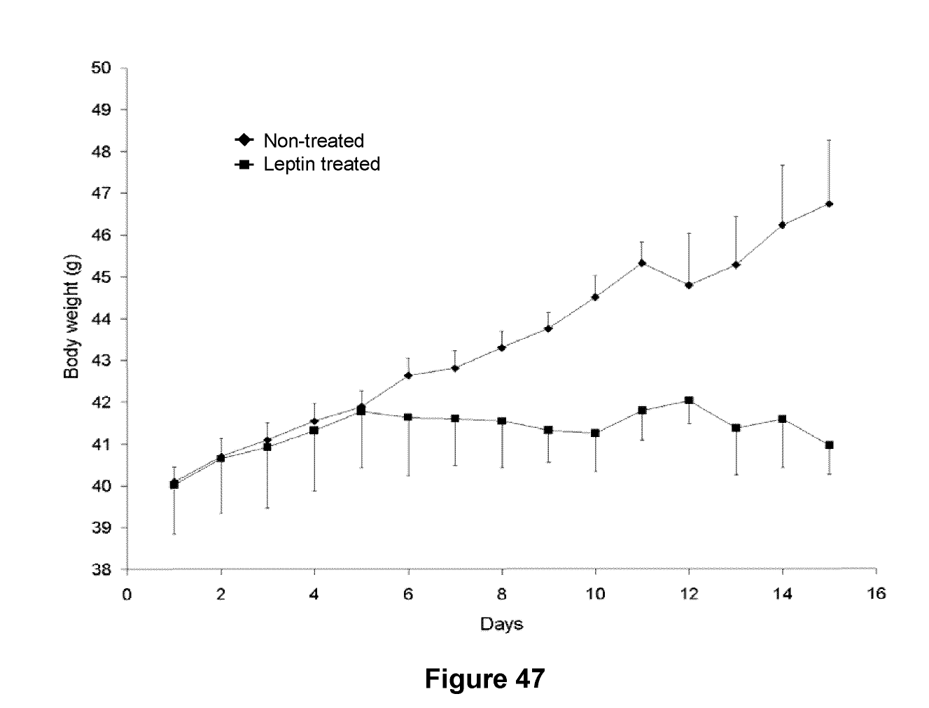

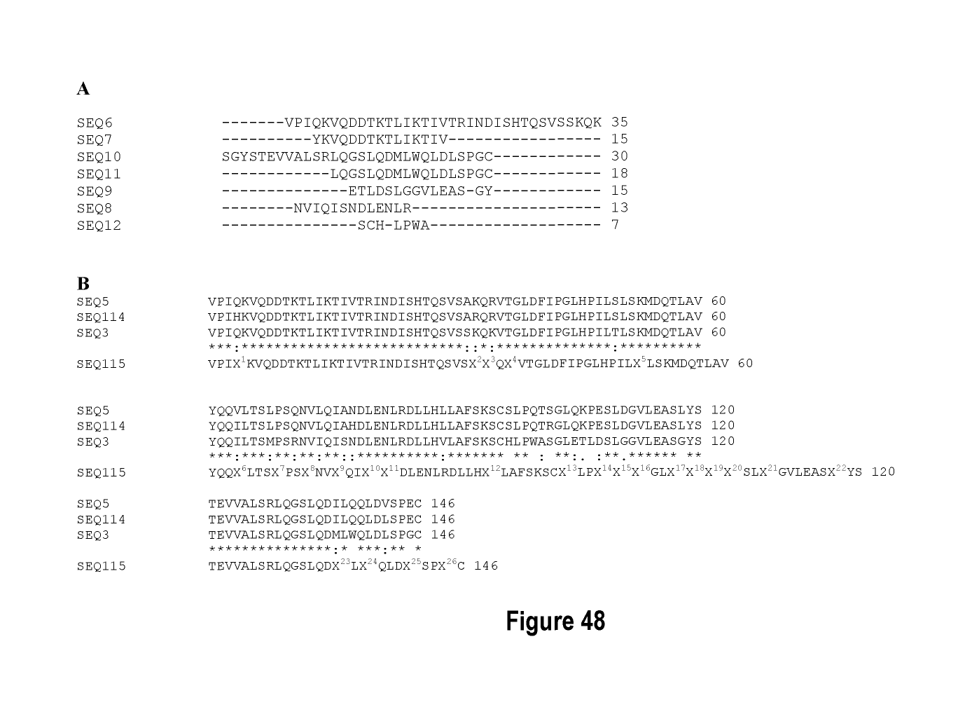

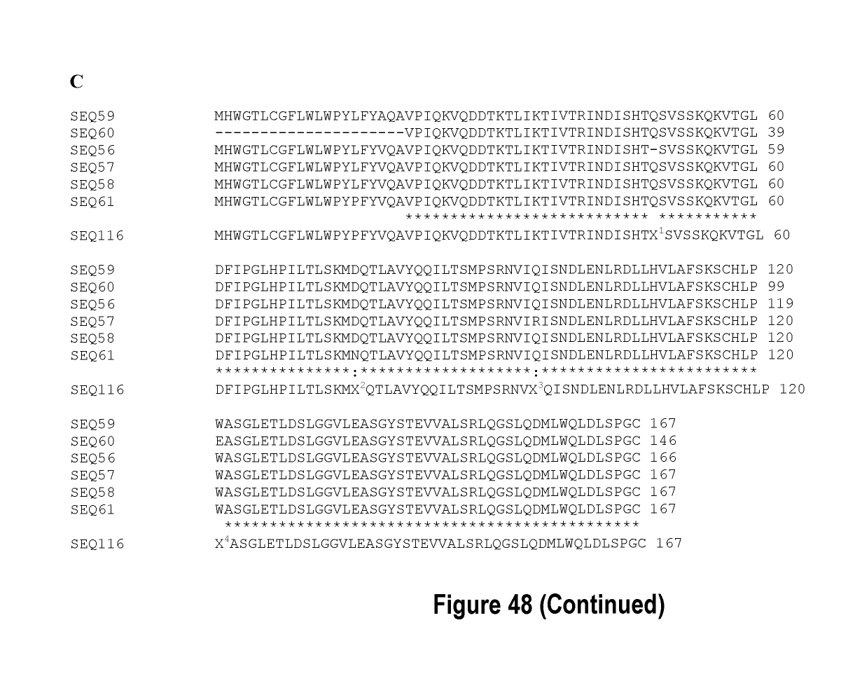

This study sought to determine the effect of oral leptin daily for maintaining or stabilizing body weight in a subject after or without weight loss. Young ob/ob mice (6 weeks old) in the period of rapid growth were selected and administered enough leptin to stabilize their body weight. Amounts of oral leptin administered in vehicle 3 were modified every two days to better achieve weight stabilization. Two series of weight control experiments were carried out on two groups of mice (n=3 for each) in December 2010 and in March 2011 (FIGS. 46A and 46B, respectively, with each line corresponding to individual mice). The mean body weight of obese ob/ob mice stabilized or not with leptin is shown in FIG. 47, in which the results are displayed as an individual growth curve for oral leptin treated and non-treated mice.

### Example 37

### Preparation of Oral Leptin Formulation Using Vehicle 5

A vehicle 5 having the following composition was prepared:

VEHICLE 5:

Sodium bicarbonate, 125 mM
Sodium deoxycholate, 30 mM
Aprotinin, 1 mg/mL
Glutamine, 500 μM

A 100 mL stock solution of Vehicle 5 was prepared by dissolving 1.05 g of NaHCO₃, 1.24 g of sodium deoxycholate in 80 mL of distilled water. The mixture was stirred until complete dissolution of all compounds. The pH of the solution was adjusted to 9 using NaOH (10 N and 1 N). The volume of the solution was then adjusted to 100 mL and the solution was kept at 4° C.

On the day of an experiment, Aprotinin (1 mg/mL); was dissolved in 10 mL of stock solution of Vehicle 5 and glutamine is added to a concentration of 500 μM. This working solution, in which leptin was dissolved, was kept up to five days at 4° C.

### Example 38

### Preparation of Oral Leptin Formulation Using Vehicle 6

A vehicle 6 having the following composition was prepared:

Vehicle 6:

Sodium bicarbonate, 125 mM
Sodium taurochlorate, 30 mM
Aprotinin, 1 mg/mL

A 100 mL stock solution of Vehicle 6 was prepared by dissolving 1.05 g of NaHCO₃, 1.24 g of sodium taurochlorate in 80 mL of distilled water. The mixture was stirred until complete dissolution of all compounds. The pH of the solution was adjusted to 9 using NaOH (10 N and 1N). The volume of the solution was then adjusted to 100 mL and the solution was kept at 4° C.

On the day of an experiment, Aprotinin (1 mg/mL); was dissolved in 10 mL of stock solution of Vehicle 6. This working solution, in which leptin was dissolved, was kept up to five days at 4° C.

Although the present invention has been described hereinabove by way of specific embodiments thereof, it can be modified, without departing from the spirit and nature of the subject invention as defined in the appended claims.

Annex 1: Human and mouse leptin sequences

>Human leptin precursor protein (signal sequence underlined)
MHWGTLCGFLWLWPYLFYVQAVPIQKVQDDTKTLIKTIVTRINDISHTQSVSSKQKVTGLDFIPGLHPILTLSKMDQ
TLAVYQQILTSMPSRNVIQISNDLENLRDLLHVLAFSKSCHLPWASGLETLDSLGGVLEASGYSTEVVALSRLQGS
LQDMLWQLDLSPGC (SEQ ID NO: 2)
>Human leptin protein (processed)
VPIQKVQDDTKTLIKTIVTRINDISHTQSVSSKQKVTGLDFIPGLHPILTLSKMDQTLAVYQQILTSMPSRNVIQISND
LENLRDLLHVLAFSKSCHLPWASGLETLDSLGGVLEASGYSTEVVALSRLQGSLQDMLWQLDLSPGC (SEQ ID NO: 3)
>Murine leptin precursor protein (signal sequence underlined)
MCWRPLCRFLWLWSYLSYVQAVPIQKVQDDTKTLIKTIVTRINDISHTQSVSAKQRVTGLDFIPGLHPILSLSKMDQ
TLAVYQQVLTSLPSQNVLQIANDLENLRDLLHLLAFSKSCSLPQTSGLQKPESLDGVLEASLYSTEVVALSRLQGS
LQDILQQLDVSPEC (SEQ ID NO: 4)
>Murine leptin protein (processed)
VPIQKVQDDTKTLIKTIVTRINDISHTQSVSAKQRVTGLDFIPGLHPILSLSKMDQTLAVYQQVLTSLPSQNVLQIAN
DLENLRDLLHLLAFSKSCSLPQTSGLQKPESLDGVLEASLYSTEVVALSRLQGSLQDILQQLDVSPEC (SEQ ID NO: 5)
>Modified murine leptin protein used in the Examples with an extra methionine
residue added at the N terminus (underlined)
MVPIQKVQDDTKTLIKTIVTRINDISHTQSVSAKQRVTGLDFIPGLHPILSLSKMDQTLAVYQQVLTSLPSQNVLQIA
NDLENLRDLLHLLAFSKSCSLPQTSGLQKPESLDGVLEASLYSTEVVALSRLQGSLQDILQQLDVSPEC (SEQ ID NO: 1)
>Modified human leptin protein used in the Examples with an extra methionine residue
added at the N terminus (underlined)
MVPIQKVQDDTKTLIKTIVTRINDISHTQSVSSKQKVTGLDFIPGLHPILTLSKMDQTLAVYQQILTSMPSRNVIQISN
DLENLRDLLHVLAFSKSCHLPWASGLETLDSLGGVLEASGYSTEVVALSRLQGSLQDMLWQLDLSPGC (SEQ ID NO: 113)
>Modified rat leptin protein used in the Examples with an extra methionine residue
added at the N terminus (underlined)
MVPIHKVQDDTKTLIKTIVTRINDISHTQSVSARQRVTGLDFIPGLHPILSLSKMDQTLAVYQQILTSLPSQNVLQIAH
DLENLRDLLHLLAFSKSCSLPQTRGLQKPESLDGVLEASLYSTEVVALSRLQGSLQDILQQLDLSPEC (SEQ ID NO: 114)

Annex 2: Human leptin fragments/variants

| Fragment/variants of | |
|---|---|
| human leptin protein | |
| (position relative to | Amino acid sequence |
| precursor protein) | (N to C terminus) |
| 22-56 | VPIQKVQDDTKTLIKTIVTRINDISH |
| | TQSVSSKQK (SEQ ID NO: 6) |
| 26-39 | YKVQDDTKTLIKTIV |
| | (SEQ ID NO: 7) |
| 93-105 | NVIQISNDLENLR (SEQ ID NO: 8) |
| 126-140 | ETLDSLGGVLEASGY |
| | (SEQ ID NO: 9) |
| 138-167 | SGYSTEVVALSRLQGSLQDMLWQLDL |
| | SPGC (SEQ ID NO: 10) |
| 150-167 | LQGSLQDMLWQLDLSPGC |
| | (SEQ ID NO: 11) |
| 116-122 | SCHLPWA (SEQ ID NO: 12) |

Annex 3: Leptin ortholods and human leptin polymorphisms

>gi|167030888|gb|ABZ05758.1| leptin [Ailuropoda melanoleuca]
MRCGPLCRFLWLWPYLSYIEAVPIRKVQDDTKTLIKTIVTRINDISHTQAVSSKQRVAGLDFIPGLHPVLSLSRMDQ
TLAIYQQILTSLHSRNVVQISNDLENLRDLLHLLASSKSCPLPRARGLESFESLGGVLEASLYSTEVVALSRLQAAL
QDMLQRLDLSPGC (SEQ ID NO: 13)
>gi|301755234|ref|XP_002913466.1| PREDICTED: leptin-like [Ailuropoda melanoleuca]
MRCGPLCRFLWLWPYLSYIEAVPIRKVQDDTKTLIKTIVTRINDISHTAVSSKQRVAGLDFIPGLHPVLSLSRMDQT
LAIYQQILTSLHSRNVVQISNDLENLRDLLHLLASSKSCPLPRARGLESFESLGGVLEASLYSTEVVALSRLQAALQ
DMLQRLDLSPGC (SEQ ID NO: 14)
>gi|47834166|gb|AAT38807.1| obese protein [Anas platyrhynchos]
VPIQKVQDDTKTLIKTIVTRINDISHTQSVSAKQRVTGLDFIPGLHPILSLSKMDQTLAVYQQVLTSLPSQNVLQIAD
DLENLRDLLHLLAFSKSCSLPQTSGLQKPESLDGVLEASLYSTEEVVALSRLQGSLQDILQQLDVSPEC (SEQ ID NO: 15)
>gi|110666863|gb|ABG81864.1| obese protein [Anguilla japonica]
GPIQKVQDDTKTLIKTIVTRINDISHTQSVSAKQRVTGLDFIPGLHPILSLSKMDQTLAVYQQVLTSLPSQNVLQIAN
DLENLRDLLHLLAFSKSCSLPQTSGLQKPESLDGVLEASLYSTEVVALSRLQGSLQDILQQLDVSPEC (SEQ ID NO: 16)
>gi|194294258|gb|ACF40216.1| leptin [Bos frontalis]
MRCGPLYRFLWLWPYLSYVEAVPISKVQDDTKTLIKTIVTRINDISHTQSVSSKQRVTGLDFIPGLHPLLSLSKMDQ
TLAIYQQILTSLPSRNVVQISNDLENLRDLLHLLAASKSCPLPQVRALESLESLGVVLEASLYSTEVVALSRLQGSL
QDMLRQLDLSPGC (SEQ ID NO: 17)
>gi|257183589|gb|ACV49867.1| leptin [Bos frontalis]
QSVSSKQRVTGLDFIPGLHPLLSLSKMDQTLAIYQQILTSLPSRNVVQISNDLENLRDLLHLLAASKSCPLPQVRAL
ESLESLGVVLEASLYSTEVV (SEQ ID NO: 18)
>gi|189214291|gb|ACD85081.1| leptin [Bos grunniens]
MRCGSLYRFLWLWPYLSYVEAVPISKVQDDTKTLIKTIVTRINDISHTQSVSSKQRVTGLDFIPGLHPLLSLSKMDQ
TLAIYQQILTSLPSRNVVQISNDLENLRDLLHLLAASKSCPLPQVRALESLESLGVVLEASLYSTEVVALSRLQGSL
QDMLRQLDLSPGC (SEQ ID NO: 19)
>gi|196122279|gb|ACG69794.1| leptin [Bos indicus]
QSVSSKQRVTGLDFIPGLHPLLSLSKMDQTLAIYQQILTSLPSRNVVQISNDLENLRDLLHLLAASKSCPLPQVRAL
ESLESLGVVLEASLYSTEVVALSRLQGSLQDMLR (SEQ ID NO: 20)
>gi|197205760|gb|ACH47996.1| leptin [Bos indicus]
SSKQRVTGLDFIPGLHPLLSLSKMDQTLAIYQQILTSLPSRNVVQISNDLENILRDLLHLLAASKSCPLPQVRALESL
ESLGVVLEASLYSTEVVALSRLQGSLQDMLRQLDLSPGC (SEQ ID NO: 21)
>gi|197205762|gb|ACH47997.1| leptin [Bos indicus]
GLDFIPGLHPLLSLSKMDQTLAIYQQILTSLPSRNVVQISNDLENLRDLLHLLAASKSCPLPQVRALESLESLGVVLE
ASLYSTEVVALSRLQGSLQDMLRQLDLSPGC (SEQ ID NO: 22)
>gi|1507748|gb|AAB06579.1| leptin [Bos taurus]
VPIQKVQDDTKTLIKTIVTRINDISHTQSVSSKQRVTGLDFIPGLHPLLSLSKMDQTLAIYQQILTSLPSRNVVQISND
LENLRDLLHLLAASKSCPLPQVRALESLESLGVVLEASLYSTEVVALSRLQGSLQDMLRQLDLSPEC (SEQ ID NO: 23)
>gi|1709435|sp|P50595.1|LEP_BOVINI RecName: Full = Leptin; AltName:
Full = Obesity factor; Flags: Precursor
MRCGPLYRFLWLWPYLSYVEAVPIRKVQDDTKILIKTIVTRINDISHTQSVSSKQRVTGLDFIPGLHPLLSLSKMDQ
TLAIYQQILTSLPSRNVVQISNDLENLRDLLHLLAASKSCPLPQVRALESLESLGVVLEASLYSTEVVALSRLQGSL
QDMLRQLDLSPGC (SEQ ID NO: 24)
>gi|1850803|emb|CAA72197.1| leptin [Bos taurus]
SHTQSVSSKQRVTGLDFIPGLHPLLSLSKMDQTLAIYQQILTSLPSRNVVQISNDLENLRDLLHLLAASKSCPLPQV
RALESLESLGVVLEASLYSTEVV (SEQ ID NO: 25)
>gi|1945613|dbj|BAA19750.1| leptin [Bos taurus]
VPIRKVQDDTKTLIKTIVTRINDISHTQSVSSKQRVTGLDFIPGLHPLLSLSKMDQTLAIYQQILTSLPSRNVVQISND
LENLRDLLHLLAASKSCPLPQVRALESLESLGVVLEASLYSTEVVALSRLQGSLQDMLRQLDLSPGC (SEQ ID NO: 26)
>gi|87196505|ref|NP_776353.2| leptin precursor [Bos taurus]
MRCGPLYRFLWLWPYLSYVEAVPICKVQDDTKILIKTIVTRINDISHTQSVSSKQRVTGLDFIPGLHPLLSLSKMDQ
TLAIYQQILTSLPSRNVVQISNDLENLRDLLHLLAASKSCPLPQVRALESLESLGVVLEASLYSTEVVALSRLQGSL
QDMLRQLDLSPGC (SEQ ID NO: 27)
>gi|197205764|gb|ACH47998.1| leptin [Bos taurus x Bos indicus]
KQRVTGLDFIPGLHPLLSLSKMDQTLAIYQQILTSLPSRNVVQISNDLENLRDLLHLLAASKSCPLPQVRALESLES
LGVVLEASLYSTEVVALSRLQGSLQDMLRQLDLSPGC (SEQ ID NO: 28)
>gi|27572983|gb|AAO19891.1|AF387813_1 leptin [Bubalus bubalis]
QSVSSKQRVTGLDFIPGLHPLLSLSKMDQTLAIYQQILTSLPSRNVVQISNDLENLRDLLHLLAASKSCPLPQVRAL
ESLESLGVVLEASLYSTEVVALSRLQGSLQDMLRQLDLSPGC (SEQ ID NO: 29)
>gi|27803698|gb|AAO21933.1| leptin [Bubalus bubalis]
QSVSSKQRVTGLDFIPGLHPLLSLSKMDQTLAIYQQILTSLPSRNVVQISNDLENLRDLLHLLAASKSCPLPQVRAL
ESLKSLGVVLEASLYSTEVVALSRLQGSLQDM (SEQ ID NO: 30)
>gi|61213764|sp|Q5J732.1|LEP_BUBBU RecName: Full = Leptin; AltName:
Full = Obesity factor; Flags: Precursor
MRCGPLYQFLWLWPYLSYVEAVPIRKVQDDTKTLIKTIVTRINDISHTQSVSSKQRVTGLDFIPGLHPLLSLSKMDQ
TLAIYQQILTSLPSRNVVQISNDLENLRDLLHLLAASKSCPLPQVRALESLESLGVVLEASLYSTEVVALSRLQGSL
QDMLRQLDLSPGC (SEQ ID NO: 31)
>gi|110558616|gb|ABG75767.1| leptin variant A [Bubalus bubalis]
KQRVTGLDFIPGLHPLLSLSKMDQTLAIYQQILTSLPSRNVVQISNDLENILRDLLHLLAASKSCPLPQVRALESLES
LGVVLEASLYSTEVVALSRLQGSLQDMLRQL (SEQ ID NO: 32)
>gi|110558618|gb|ABG75768.1| leptin variant B [Bubalus bubalis]
KQRVTGLDFIPGLHPLLSLSKMDQTLAIYQQILTSLPSRNVVQISNDLENLRDLLHLLAASKSCPLPQVRALESLES
LGVVLEASLYSTEVVALSRLQGSLQDMLQQL (SEQ ID NO: 33)
>gi|158828374|gb|ABW81205.1| leptin [Bubalus bubalis]
QRVTGLDFIPGLHPLLSLSKMDQTLAIYQQILTSLPSRNVVQISNIDLENLRDLLHLLAASKSCPLPQVRALESLESL
GVVLEASLYSTEVVALSRLQGSLQDMLRQLDLSPGC (SEQ ID NO: 34)
>gi|158939085|gb|ABW83993.1| leptin [Bubalus bubalis]
QRVTGLDFIPGLHPLLSLSKMDQTLAIYQQILTSLPSRNVVQISNDLENLRDLLHLLAASKSCPLPQVRALESLESL
GVVLEASLYSTEVVALSRLQGSLQDMLQQLDLSPGC (SEQ ID NO: 35)
>gi|196122277|gb|ACG69793.1| leptin [Bubalus bubalis]
QSVSSKQRVTGLDFIPGLHPLLGLSKMDQTLAIYQQILTSLPSRNVVQISNDLENLRDLLHLLAASKSCPLPQVRAL
ESLESLGVVLEASLYSTEVVALSRLQGSLQDMLR (SEQ ID NO: 36)
>gi|296210713|ref|XP_002752099.1| PREDICTED: leptin-like [Callithrix jacchus]
MRWGCLCRFLWLWACLSYTQAVPIQRVQDDTKTLIKTIIARINDLSHTQSVSPRQRVTGLEFIPGFHSDLSFSKMD
EILATYQQIVISLPSGNMIQISNDLENLRALLHLLAASKSCHLPWASGLENLANLGGVLEVSLYSTEVVALSRLRGTL
KDILQQLDLGPAC (SEQ ID NO: 37)
>gi|29825695|gb|AAO91910.1| leptin [Camelus dromedarius]
QSVSSKQRVTGLDFIPGLHPLLSLSKMDQTLAIYQQILTSLPSRNVVQISNDLESLRDLLHLLAASKSCPLPQVRAL
ESLESLGVVLEASLYSTEVVALSWLQGSLQDM (SEQ ID NO: 38)
>gi|2072094|gb|AAB53654.1| leptin [Canis lupus familiaris]
VPIRKVQDDIKTLIKTIVARINDISHTQSVSSKQRVAGLDFIPGLQPVLSLSRMDQTLAIYQQILNSLHSRNVVQISN
DLENLRDLLHLLASSKSCPLPRARGLETFESLGGVLEASLYSTEVVALSRLQAALQDMLRRLDLSPGC (SEQ ID NO: 39)
>gi|20502046|gb|AAM21762.1| leptin [Canis lupus familiaris]
SVSSKQRVAGLDFIPGLQPVLSLSRMDQTLAIYQQILNSLHSRNVVQISNDLENLRDLLHLLASSKSCPLPRARGL
ETFESLGGVLEASLYSTEVVALSRLQAALQDMLRRLDLSPGC (SEQ ID NO: 40)
>gi|50978738|ref|NP_001003070.1| leptin precursor [Canis lupus familiaris]
MRCGPLCRFLWLWPYLSCVEAVPIRKVQDDTKTLIKTIVARINDISHTQSVSSKQRVAGLDFIPGLQPVLSLSRMD
QTLAIYQQILNSLHSRNVVQISNDLENLRDLLHLLASSKSCPLPRARGLETFESLGGVLEASLYSTEVVALNRLQAA
LQDMLRRLDLSPGC (SEQ ID NO: 41)
>gi|14317955|gb|AAK59872.1| leptin [Capra hircus]
PGLHXVLSLSKMDQTLAIYQQILTSLPSRNVIQISNDLENLRDLLHLLASSKSCPLPQARALETLEXLGGVLEASLYS
TEVVALTRLKGAFXDMLRKLDLALVVEA (SEQ ID NO: 42)
>gi|97071731|sp|Q257X2.1|LEP_CAPHI RecName: Full = Leptin; AltName:
Full = Obesity factor; Flags: Precursor
MRCGPLYRFLWLWPYLSYVEAVPIRKVQDDTKTLIKTIVTRINDISHTQSVSSKORVTGLDFIPGLHPLLSLSKMDQ
TLAIYQQILASLPSRNVIQISNDLENLRDLLHLLAASKSCPLPQVRALESLESLGVVLEASLYSTEVVALSRLQGSLQ
DMLRQLDLSPGC (SEQ ID NO: 43)
>gi|157804567|gb|ABV79899.1| leptin [Capra hircus]
MVPIRKVQDDTKTLIKTIVTRINDISHTQSVSSKQRVTGLDFIPGLHPLLSLSKMDQTLAIYQQILASLPSRNVIQISN
DLENILRDLLHLLAASKSCPLPQVRALESLESLGVVLEASLYSTEVVALSRLQGSLQDMLRQLDLSPGC (SEQ ID NO: 44)
>gi|48526405|gb|AAT45399.1| obese protein [Channa argus]
VPIQEVQDDTKTLIKTIVTRINDISHTQSVSAKQRVTGLDFIPGLHPILSLSKMDQTLAVYQQVLTSLPSQNVLQIAN
DLKNLRDLLHLLAFSKSCSLPQTSGLQKPESLDGVLEASLYSTEVVALSRLQXSLQDILQQLDVSPEC (SEQ ID NO: 45)
>gi|48526395|gb|AAT45394.1| obese protein [Ctenopharyngodon idella]
VPIQKVQDDTKTLIKTIVTRINDISHTQSVSAKQRVTGLDFIPGLHPILSLSKMDQTLAVYQQVLTSLPSQNVLQIAN
DLENLRDLLHLLAFSKSCSLPQTSGLQKPESLDGVLEASLYSTEVVALSRLQGSLQDILQQLDVSPEC (SEQ ID NO: 46)
>gi|47716909|gb|AAT37636.1| leptin precursor [Culter sp. TP-2004]
VPIQKVQDDSKTLIKTIVTRINDISHTQSVSAKQRVTGLDFIPGLHPILSLSKMDQTLAVYQQVLTSLPSQNVLQIAN
GLKNLRDLLHLLAFSKSCSLPQTSGLQKPESLDGVLEASLYSTEVVALSRLQGSLQDILQQLDVSPEC (SEQ ID NO: 47)
>gi|47498581|gb|AAT28186.1| obese protein [Cyprinus carpio]
VPIQKVQDDTKTLIKTIVTRINDISHTQSVSAKQRVTGLDFIPGLHPILSLSKMDQTLAVYQQVLTSLPSQNVLQIAN
DLKNLRDLLHLLAFSKSCSLPQTSGLQKPESLDGVLEASLYSTEVVALSRLQGSLQDILQQLDVSPEC (SEQ ID NO: 48)
>gi|5815453|gb|AAD52679.1| leptin [Equus caballus]
DTKTLIKTIVTRINDISHTQSVSSKQRVTGLDFIPGLHPVLSLSKMDQTLAIYQQILTSLPSRNVIQISNDLENLRDLLH
LLASSKSCPLPQARGLETL (SEQ ID NO: 49)
>gi|57015328|sp|Q9TU09.2|LEP_HORSE RecName: Full = Leptin; AltName:
Full = Obesity factor; Flags: Precursor
LWLWPYLFFIEAVPIRKVQDDTKTLIKTIVTRINDISHTQSVSSKQRVTGLDFIPGLHPVLSLSKMDQTLAIYQQILTS
LPSRNVIQISNDLENLRDLLHLLASSKSCPLPQARGLETLASLGGVLEASLLLHRGGSPEQAAGVS (SEQ ID NO: 50)
>gi|255653078|ref|NP_001157452.1| leptin [Equus caballus]
MHCGPLCQFLWLWPYLFFIEAVPIRKVQDDTKTLIKTIVTRINDISHTQSVSSKQRVTGLDFIPGLHPVLSLSKMDQ
TLAIYQQILTSLPSRNVIQISNDLENLRDLLHLLASSKSCPLPQARGLETLASLGGVLEASLYSTEVVALSRLQGSLQ
DMLQQLDLSPGC (SEQ ID NO: 51)
>gi|57619023|ref|NP_001009850.1| leptin precursor [Felis catus]
MLCGPLCRFLWLWPYLSYVEAVPIRKVQDDTKTLIKTIVTRINDISHTQSVSSKQRVAGLDFIPGLHPVLSLSKMDQ
TLAIYQQILTGLPSRNVVQISNDLENLRDLLHLLASSKNCPLPRARGLETLESLGGALEASLYSTEVVALSRLQASL
QDMLWRLDLSPGC (SEQ ID NO: 52)
>gi|3024234|sp|O42164.1|LEP_CHICK RecName: Full = Leptin; AltName:
Full = Obesity factor; Flags: Precursor
MCWRPLCRLWSYLVYVQAVPCQIFQDDTKTLIKTIVTRINDISHTSVSAKQRVTGLDFIPGLHPILSLSKMDQTLAV
YQQVLTSLPSQNVLQIANDLENLRDLLHLLAFSKSCSLPQTSGLQKPESLDGVLEASLYSTEVVALSRLQGSLQDI
LQQLDISPEC (SEQ ID NO: 53)
>gi|2498683|sp|Q95189.1|LEP_GORGO RecName: Full = Leptin; AltName: Full = Obesity factor
VPIQKVQDDTKILIKTIVTRISDISHTQSVSSKQKVTGLDFIPGLHPILTLSKMDQTLAVYQQILTSMPSRNMIQISND
LENLRDLLHVLAFSKSCHLPWASGLETLDSLGGVLEASGYSTEVVALSRLQGSLQDMLWQLDLSPGC (SEQ ID NO: 54)
>gi|61213765|sp|Q706D0.1|LEP_HALGR RecName: Full = Leptin; AltName:
Full = Obesity factor; Flags: Precursor
MRCGSLCRFLWLWSCLPYIEAMPIQRVQDDTKTLIKTIITRINDISPPQGVCSRPRVAGLDFIPRVQSVRILSGMD
QILATYQQILTSLQSRSVVQIANDLANLRALLRLLASAKSCPVPRARGSDTIKGLGNVLRASVHSTEVVALSRLKAA
LQDMLRQLDRNPGC (SEQ ID NO: 55)
>gi|904212|dbj|BAA08448.1| obese [Homo sapiens]
MHWGTLCGFLWLWPYLFYVQAVPIQKVQDDTKTLIKTIVTRINDISHTSVSSKQKVTGLDFIPGLHPILTLSKMDQT
LAVYQQILTSMPSRNVIQISNDLENLRDLLHVLAFSKSCHLPWASGLETLDSLGGVLEASGYSTEVVALSRLQGSL
QDMLWQLDLSPGC (SEQ ID NO: 56)
>gi|2267088|gb|AAB63507.1| obese protein [Homo sapiens]
MHWGTLCGFLWLWPYLFYVQAVPIQKVQDDTKTLIKTIVTRINDISHTQSVSSKQKVTGLDFIPGLHPILTLSKMDQ
TLAVYQQILTSMPSRNVIRISNDLENLRDLLHVLAFSKSCHLPWASGLETLDSLGGVLEASGYSTEVVALSRLQGS
LQDMLWQLDLSPGC (SEQ ID NO: 57)
>gi|4557715|ref|NP_000221.1| leptin precursor [Homo sapiens]
MHWGTLCGFLWLWPYLFYVQAVPIQKVQDDTKTLIKTIVTRINDISHTQSVSSKQKVTGLDFIPGLHPILTLSKMDQ
TLAVYQQILTSMPSRNVIQISNDLENLRDLLHVLAFSKSCHLPWASGLETLDSLGGVLEASGYSTEVVALSRLQGS
LQDMLWQLDLSPGC (SEQ ID NO: 58)
>gi|46854316|gb|AAH69323.1| Leptin [Homo sapiens]
MHWGTLCGFLWLWPYLFYAQAVPIQKVQDDTKTLIKTIVTRINDISHTQSVSSKQKVTGLDFIPGLHPILTLSKMDQ
TLAVYQQILTSMPSRNVIQISNDLENLRDLLHVLAFSKSCHLPWASGLETLDSLGGVLEASGYSTEVVALSRLQGS
LQDMLWQLDLSPGC (SEQ ID NO: 59)
>gi|157830127|pdb|1AX8|A Chain A, Human Obesity Protein, Leptin
VPIQKVQDDTKTLIKTIVTRINDISHTQSVSSKQKVTGLDFIPGLHPILTLSKMDQTLAVYQQILTSMPSRNVIQISND
LENLRDLLHVLAFSKSCHLPEASGLETLDSLGGVLEASGYSTEVVALSRLQGSLQDMLWQLDLSPGC (SEQ ID NO: 60)
>gi|189069297|dbj|BAG36329.1| unnamed protein product [Homo sapiens]
MHWGTLCGFLWLWPYPFYVQAVPIQKVQDDTKTLIKTIVTRINDISHTQSVSSKQKVTGLDFIPGLHPILTLSKMNQ
TLAVYQQILTSMPSRNVIQISNDLENLRDLLHVLAFSKSCHLPWASGLETLDSLGGVLEASGYSTEVVALSRLQGS
LQDMLWQLDLSPGC (SEQ ID NO: 61)
>gi|48526399|gb|AAT45396.1| obese protein [Hypophthalmichthys nobilis]
VPIQKVQDDSKTLIKTIVTRINDISHTQSVSAKQRVTGLDFIPGLHPILSLSKMDQTLAVYQQVLTSLPSQNVLQIAN
GLKNLRDLLHLLAFSKSCSLPQTSGLQKPESLDGVLEASLYSTEVVALSRLQXSLQDILQQLDVSPEC (SEQ ID NO: 62)
>gi|118419971|gb|ABK88255.1| leptin [Lagenorhynchus albirostris]
VQDDTKTLIKTIVTRINDISHTRSVSSKQRVTGLDFIPGLTPVLSLSKMDQTLTIYQQILTSLPSRNVIQISNDLENLR
DLLHLLASSKSCPLPQARALETLESLGGVLEASLYSTEVVAQS (SEQ ID NO: 63)
>gi|81294756|emb|CAJ43201.1| Leptin [Leptonychotes weddellii]
VQDDTKTLIRTIIARINDISQPGVCSRPRVAGLDFIPGPQSVRTLSGMNQMLAIYQQILTSLHSRSVVQIANDLANLR
DLLHLLASAKSCPLPRARGLENIKSLRDVLKASVHSTEVVALSRLRAALQGMLRQLDRNPGC (SEQ ID NO: 64)
>gi|119395629|gb|ABL74887.1| leptin [Lepus oiostolus]
MRCGPLCRLLWLWPCLSCVPAVPMRKVQDDTKTLIKTIVTRISDISHTQSVSSRQRVVGLDFIPGLHPNLSLSTMD
QTLAIYQQILTSLPSRNVIQIANDLENLRDLLHLLALSKSCPLPRASGLETLEGLGGVLEASLYSTEVVALSRLQGSL
QAMLQQLDLGPGC (SEQ ID NO: 65)
>gi|112363109|ref|NP_001036220.1| leptin precursor [Macaca mulatta]
MYWRTLWGFLWLWPYLFYIQAVPIQKVQSDTKTLIKTIVTRINDISHTQSVSSKQRVTGLDFIPGLHPVLTLSQMD
QTLAIYQQILINLPSRNVIQISNDLENLRDLLHLLAFSKSCHLPLASGLETLESLGDVLEASLYSTEVVALSRLQGSL
QDMLWQLDLSPGC (SEQ ID NO: 66)
>gi|48526403|gb|AAT45398.1| obese protein [Megalobrama amblycephala]
VPIQKVQDDTKTLIKTIVTRINDISHTQSVSAKQRVTGLDFIPGLHPILSLSKMDQTLAVYQQVVTSLPSQNVLQIAN
DLKNLRDLLHLLAFSKSCSLPQTSGLQKPESLDGVLEASLYSTEVVALSRLQGSLQDILQQLDVSPEC (SEQ ID NO: 67)
>gi|13631501|sp|O93416.1|LEP_MELGA RecName: Full = Leptin; AltName: Full = Obesity factor
VPCQIFQDDTKTLIKTIVTRINDISHTSVSAKQSVTGLDFIPGLHPILSLSKMDQTLAVYQQVLTSLPSQNVLQIANDL
ENLRDLLHLLAFSKSCSLPQTSGLHKPESLDGVLEALLYSTEVVALSRLQGSLQDILQQLDISPEC (SEQ ID NO: 68)
>gi|126340669|ref|XP_001366398.1| PREDICTED: similar to leptin [Monodelphis domestica]
MHCVALCSFLWLCHHLYYTQAVPIRKVQDDTKTLTKTIITRINDISHMYSISAKQRVTGLDFIPGLHPFQSLSDMDQ
TLAIYQQILSNLSSRNMVQISNDLENLRDLLHLLGSLKSCPFDEADGLSSLGNLEGVMEASLYSTEVVILTRLQKSL
YGMLQQLDLIHGC (SEQ ID NO: 69)
>gi|6678678|ref|NP_032519.1| leptin precursor [Mus musculus]
MCWRPLCRFLWLWSYLSYVQAVPIQKVQDDTKTLIKTIVTRINDISHTQSVSAKQRVTGLDFIPGLHPILSLSKMDQ
TLAVYQQVLTSLPSQNVLQIANDLENLRDLLHLLAFSKSCSLPQTSGLQKPESLDGVLEASLYSTEVVALSRLQGS
LQDILQQLDVSPEC (SEQ ID NO: 70)
>gi|6356675|gb|AAL16404.1| leptin [Myotis lucifugus]
FYAEAAPIQKVQDDTKTLIKTIVTRINDISHTRSVSSRQRVTGLDFIPGLHPILSLSRMDQTLAIYQQILTSLPSGNVL
QISNDLENLRDLLHLLASSNSCPFPRTRSLKTLEGLDDALEASL (SEQ ID NO: 71)
>gi|192293825|gb|ACE87887.1| putative leptin [Neovison vison]
MLCGPLCRFLWLWPYLSYVEAVPIRKVQDDTKTLIKTIVTRISDISHTAVSSKQRVAGLDFIPGLHPVLSLSRMDQT
LAIYQQILTSLHSRNVIQISNDLENLRDLLRLLASSKSCPLPRARGLESFESLGGVLEASLYSTEVVALSRLQAALQ
DMLGRLDLSPGC (SEQ ID NO: 72)
>gi|256692869|gb|ACV13199.1| leptin [Notomys alexis]
MCWRPLCWFLWLWSYLSYVQALPVQKVQDDTKTLIKTIATRINDISHTQSVSAKQRVTGLDFIPGLHPILSLSKMD
QTLVVYQQILTSLPSGNVLQIANDLENLRDLLRLAFSKSCSLPQTSGLQKPESLDGVLEASLYSTEVVALSRLQG
FLQDILQQLDLTPEC (SEQ ID NO: 73)
>gi|20502050|gb|AAM21764.1| leptin [Nyctereutes procyonoides procyonoides]
SVSSKQRVAGLDFIPGLQPVLSLSRMDQTLAIYQQILTSLHSRNVVQISNDLENLRDLLHLLASSKSCPLPRARGLE
TFESLGGVLEASLYSTEVVALSRLQAALQDMLRRLDLSPGC (SEQ ID NO: 74)
>gi|119395625|gb|ABL74885.1| leptin [Ochotona cansus]
MRCGPLRQLLWLWPCLCVQAVSIWKVRDDTKTLIKTIVIRISDISHTHAVSSKQRITGLDFIPALHPNLSLSKMDQ
TLVLYKHILTSLPSRNVVQIANDLENLRDLLHLLAASQGCPPPRASDLESLNSLESILEASLYSTEVVALSRLQGSL
HEMLQQLDIGPGC (SEQ ID NO: 75)
>gi|82780246|gb|ABB90403.1| leptin [Ochotona curzoniae]
MRCGPLCQLLWLWPCLLCVQAVSIWKVRDDTKTLIKTILTRISDISHTHAVSSKQRITGLDFIPALHPNLSLSKMDQ
TLVLYKHILTSLPSRNVVQIANDLENLRDLLHLVAASQGCPPPRASDLESLNSLESILEASLYSTEVVALSRLQGSL
HEMLQQLDIGPGC (SEQ ID NO: 76)
>gi|119395619|gb|ABL74882.1| leptin [Ochotona dauurica]
MRCGPLCQLLWLWPCLLCVQAVSIWKVRDDTKTLIKTIVTRISDISHTHAVSSKQRITGLDFIPALHPNLSLSKMDQ
TLVLYKHILTSLPSRNVVQIANDLENLRDLLHLLAASQGCPPPRASDLESLNSLESILEASLYSTEVVALSRLQGSL
HEMLQQLDIGPGC (SEQ ID NO: 77)
>gi|124558610|gb|ABN13964.1| leptin [Ochotona dauurica bedfordi]
MRCGPLCQLLWLWPCLCVQAVSIWKVRDDTKTLIKTIVTRISDISHTHAVSSKQRITGLDLIPALHPNLSLSKMDQ
TLVLYKHILTSLPSRNVVQIANDLENLRDLLHLLAASQGCPPPRASDLESLNSLESILEASLYSTEVVALSRLQGSL
HEMLQQLDIGPGC (SEQ ID NO: 78)
>gi|119395621|gb|ABL74883.1| leptin [Ochotona thomasi]
MRCGPLCQLLWLWPCLLCVQAVSIWKVRDDTKTLIKTILTRISDISHTHAVSSKQRITGLDFTPALHPNLSLSKMDQ
TLVLYKHILTSLPSRNVVQIADDLENLRDLLHLVAASQGCPPPRASDLESLNSLESILEASLYSTEVVALSRLQGSL
HEMLQQLDIGPGC (SEQ ID NO: 79)
>gi|251823960|ref|NP_001156541.1| leptin [Oryctolagus cuniculus]
MRCGPLCQLLWLWPCLSCVPAVPMRKVQDDTKTLIKTIVTRISDISHTQSVSSRQRVVGLDFIPGLHPNLSLSTMD
QTLAIYQQILASLPSRNVIQIANDLENLRDLLHLLASSKSCPLPRASGLETLEGLGGVLEASLYSTEVVALSRLQGFL
QAMLQQLDLGPGC (SEQ ID NO: 80)
>gi|1480716|gb|AAB51033.1| leptin [Ovis aries]
DTKTLIKTIVTRINDISHTQSVSSKQRVTGLDFIPGLHPLLSLSKMDQTLAIYQQIHASLPSRNVIQISNDLENLRDLL
HLLAGSKSCPLPQVRALESLESLGVVLEASLYSTEVLA (SEQ ID NO: 81)
>gi|3041703|sp|Q28603.2|LEP_SHEEP RecName: Full = Leptin; AltName: Full = Obesity factor
VPIRKVQDDTKTLIKTIVTRINDISHTQSVSSKQRVTGLDFIPGLHPLLSLSKMDQTLAIYQQILASLPSRNVIQISNDL
ENLRDLLHLLAASKSCPLPQVRALESLESLGVVLEASLYSTEVVALSRLQGSLQDMLRQLDLSPGC (SEQ ID NO: 82)
>gi|62512365|gb|AAX39721.1| leptin [Ovis aries]
TGLDFIPGLHPLLSLSKMDQTLAIYQQILASLPSRNVIQISNDLENLRDLLHLLAASKSCPLPQVRALESLESLGVVL
EASLYSTEVVALSRLQGSLQDMLRQLDLSPGC (SEQ ID NO: 83)
>gi|146199423|gb|ABQ09501.1| leptin [Ovis aries]
QSVSSKQRVTGLDFIPGLHPLLSLSKMDQTLAIYQQILASLPSRNVIQISNDLENLRDLLHLLAASKSCPLPQVRAL
ESLESLGVVLEASLYSTEVVALSRLQGSLQDMLRQLDLSPGC (SEQ ID NO: 84)
>gi|146199425|gb|ABQ09502.1| leptin [Ovis aries]
QSVSSKQRVTGLDFIPGLHPLLSLSKMDQTLAIYQQILASLPSRNVIQISNDLENLRDLLHLLAASKSCPLPQVRAL
ESLESLGVVLEASLYSTELVALSRLQGSLQDMLRQLDLSPGC (SEQ ID NO: 85)
>gi|146199427|gb|ABQ09503.1| leptin [Ovis aries]
QSVSSKQRVTGLDFIPGLHPLLSLSKMDQTLAIYQQILASLPSRNVIQISNDLENLRDLLHLLAASKSCPLQQVRAL
ESLESLGVVLEASLYSTEVVALSRLQGSLQDMLRQLDLSPGC (SEQ ID NO: 86)
>gi|146199429|gb|ABQ09504.1| leptin [Ovis aries]
QSVSSKQRVTGLDFIPGLHPLLSLSKMDQTLAIYQQILASLPSRNVIQISNDLENLQDLLHLLAASKSCPLPQVRAL
ESLESLGVVLEASLYSTEVVALSRLQGSLQDMLRQLDLSPGC (SEQ ID NO: 87)
>gi|306480803|emb|CBX02943.1| leptin product [Ovis aries]
QDDTKTLIKTIVTRINDISHTQSVSSKQRVTGLDFIPGLHPLLSLSKMDQTLAIYQQILASLPSRNVIQISNDLENLRD
LLHLLAASKSCPLPQVRALESLESLGVVLEASLYSTELVALSRLQGSLQDMLRQLDLSPGC (SEQ ID NO: 88)
>gi|306480805|emb|CBX02944.1| leptin product [Ovis aries]
QDDTKTLIKTIVTRINDISHTQSVSSKQRVTGLDFIPGLHPLLSLSKMDQTLAIYQQILASLPSRNVIQISSDLENLRD
LLHLLAASKSCPLPQVRALESLESLGVVLEASLYSTEVVALSRLQGSLQDMLRQLDLSPGC (SEQ ID NO: 89)
>gi|306480807|emb|CBX02945.1| leptin product [Ovis aries]
QDDTKTLIKTIVTRINDISHTSVSSKQRVTGLDFIPGLHPLLSLSKMDQTLAIYQQILASLPSRNVIQISNDLENLRDLL
HLLAASKSCPLPQVRALESLESLGVVLEASLYSTELVALSRLQGSLQDMLRQLDLSPGC (SEQ ID NO: 90)
>gi|306480809|emb|CBX02946.1| leptin product [Ovis aries]
QDDTKTLIKTIVTRINDISHTQSVSSKQRVTGLDFIPGLHPLLSLSKMDQTLAIYQQILASLPSRNVIQISNDLENLQD
LLHLLAASKSCPLPQVRALESLESLGVVLEASLYSTEVVALSRLQGSLQDMLQQLDLSPGC (SEQ ID NO: 91)
>gi|3024231|sp|O02750.1|LEP_PANTR RecName: Full = Leptin; AltName: Full = Obesity factor
VPIQKVQDDTKTLIKTIVTRINDISHTQSVSSKQKVTGLDFIPGLHPILTLSKMDQTLAVYQQILTSMPSRNMIQISND
LENLRDLLHVLAFSKSCHLPWASGLETLDSLGGVLEASGYSTEVVALSRLQGSLQDMLWQLDLSPGC (SEQ ID NO: 92)
>gi|302488569|ref|NP_001180601.1| leptin [Pan troglodytes]
MHWGTLCGFLWLWPYLFYVQAVPIQKVQDDTKTLIKTIVTRINDISHTQSVSSKQKVTGLDFIPGLHPILTLSKMDQ
TLAVYQQILTSMPSRNMIQISNDLENLRDLLHVLAFSKSCHLPWASGLETLDSLGGVLEASGYSTEVVALSRLQGS
LQDMLWQLDLSPGC (SEQ ID NO: 93)
>gi|61213767|sp|Q706D1.1|LEP_PHOVI RecName: Full = Leptin; AltName:
Full = Obesity factor; Flags: Precursor
MRCGSLCRFLWLWSCLSYIEAVPIQRVQDDTKTLIKTIITRINDISPPQGVCSRPRVAGLDFIPRVQSVRTLSGMDQ
ILATYQQILTSLQSRSVVQIANDLANLRALLRLLASAKSCPVPRARGSDTIKGLGNVLRASVHSAEVVALSRLKAAL
QDMLRQLDRNPGC (SEQ ID NO: 94)
>gi|81294750|emb|CAJ43198.1| leptin [Phocoena phocoena]
MRCGPLCRFLWLWPYLSYIEAVPIRKVQDDTKTLIKTIVTRINDISHTQSVSSKQRVTGLDFIPGLTPVLSLSKMDQ
TLAIYQQILTSLPSRNVIQISNDLENLRDLLHLLASSKSCPLPQARALETLESLGGVLEAS (SEQ ID NO: 95)
>gi|66863218|emb|CAI99387.1| leptin [Phodopus campbelli]
VQDDTKTLIKTIVTRINDISHTQSVSAKQRVTGLDFIPGLHPILSLSKMDQTLAVYQQILTSLPSRNMVQISNDLENL
RDLLHLLASSKSCSLPQTSELQKLESLDGVLEASLYSTEV (SEQ ID NO: 96)
>gi|297681426|ref|XP_002818456.1| PREDICTED: leptin-like [Pongo abelii]
MHWGTLCGFLWLWPYLFYVQAVPIQKVQDDTKTLIKTVITRINDISHTQSVSSKQKVTGLDFIPGLHPILTLSKMDQ
TLAVYQQILTSMPSRNVIQISNDLENLRDLLHVLAFSKSCHLPWASGLETLDRLGGVLEASGYSTEVVALSRLQRS
LQDMLWQLDLSPGC (SEQ ID NO: 97)
>gi|2498685|sp|Q95234.1|LEP_PONPY RecName: Full = Leptin; AltName: Full = Obesity factor
VPIQKVQDDTKTLIKTVITRINDISHTQSVSSKQKVTGLDFIPGLHPILTLSKMDQTLAVYQQILTSMPSRNVIQISND
LENLRDLLHVLAFSKSCHLPWASGLETLDRLGGVLEASGYSTEVVALSRLQRSLQDMLWQLDLSPGC (SEQ ID NO: 98)
>gi|1215740|gb|AAC52514.1| leptin [Rattus norvegicus]
SYLSYVQAVPIHKVQDDTKTLIKTIVTRINDISHTQSVSARQRVTGLDFIPGLHPILSLSKMDQTLAVYQQILTSLPSQ
NVLQIAHDLENLRDLLHLLAFSKSCSLPQTRGLQKPESLDGVLEASLYSTEVVALSRLQGSLQDILQQLDVSPEC
(SEQ ID NO: 99)
>gi|6981148|ref|NP_037208.1| leptin precursor [Rattus norvegicus]
MCWRPLCRFLWLWSYLSYVQAVPIHKVQDDTKTLIKTIVTRINDISHTQSVSARQRVTGLDFIPGLHPILSLSKMDQ
TLAVYQQILTSLPSQNVLQIAHDLENLRDLLHLLAFSKSCSLPQTRGLQKPESLDGVLEASLYSTEVVALSRLQGSL
QDILQQLDLSPEC (SEQ ID NO: 100)
>gi|48526401|gb|AAT45397.1| obese protein [Silurus asotus]
VPIQKVQDDTKTLIKTIVTRINDISHTQSVSAKQTLTGLDFIPGLHPILSLSKMDQTLAVYQQVLTSLPSQNVLQIAND
LKNLRDLLHLLAFSKSCSLPQTSGLQKPESLDGVLEASLYSTEVVALSRLQGSLQDILQQLDVSPEC (SEQ ID NO: 101)
>gi|13631506|sp|Q9XSW9.1|LEP_SMICR RecName: Full = Leptin; AltName:
Full = Obesity factor; Flags: Precursor
MHCVPLFCFLWFCHHLYYSQAVPIRKVQDDTKTLTKTIITRINDISHMYSISAKQRVTGLDFIPGLHPFQSLSDMDQ
TLAIYQQILSNLSSRNMVQISNDLENLRDLLHLLGSLKSCPFDEAGGLSALGNLEGVMEASLYSTEVVTLTRLQKS
LYVMLQQLDLIHGC (SEQ ID NO: 102)
>gi|1589742|gb|AAC48641.1| leptin [Sus scrofa]
YLSYVEGPPIWRVQDDTKTLIKTIVTRISDISHMQSVSSKQRVTGLDFIPGLHPVLSLSKMDQTLAIYQQILTSLPSR
NVIQLSNDLENLRDLLHL (SEQ ID NO: 103)
>gi|2801401|gb|AAB97308.1| leptin [Sus scrofa]
MRCGPLCRFLWLWPYLSYVEAVPIWRVQDDTKTLIKTIVTRISDISHMQSVSSKQRVTGLDFIPGLHPVLSLSKMD
QTLAIYQQILTSLPSRNVIQISNDLENLRDLLHLLASSKSCPLPQRRALETLESLGGVLEASLYSTEVVALSRLQGAL
QDMLRQLDLSPGC (SEQ ID NO: 104)
>gi|19073391|gb|AAL84792.1| leptin [Sus scrofa]
MRCGPLCRFLWLWPYLSYVEAVPIWRVQDDTKTLIKTIVTRISDISHMQSVSSKQRVTGLDFIXGLHPVLSLSKMD
QTLAIYQQILTSLPSRNVIQISNDLENLRDLLHLLASSKSCPLPQARALETLESLGGVLEASLYSTEVVALSRLQGAL
QDMLRQLDLSPGC (SEQ ID NO: 105)
>gi|55741433|ref|NP_999005.1| leptin precursor [Sus scrofa]
MRCGPLCRFLWLWPYLSYVEAVPIWRVQDDTKTLIKTIVTRISDISHMQSVSSKQRVTGLDFIPGLHPVLSLSKMD
QTLAIYQQILTSLPSRNVIQISNDLENLRDLLHLLASSKSCPLPQARALETLESLGGVLEASLYSTEVVALSRLQGAL
QDMLRQLDLSPGC (SEQ ID NO: 106)
>gi|193794874|gb|ACF21597.1| leptin precursor [Sus scrofa]
VPIWRVQDDIKTLIKTIVTRISDISHMQSVSSKQRVTGLDFIPGLHPVLSLSKMDQTLAIYQQILTSLPSRNVIQISNID
LENLRDLLHLLASSKSCPLPQARALETLESLGGVLEASLYSTEVVALSRLQGALQDMLRQLDLSPGC (SEQ ID NO: 107)
>gi|243010668|gb|ACS94424.1| leptin [Sus scrofa]
MRCGPLCRFLWLWPYLSYVEAVPIWRVQDDTKTLIKTIVTRISDISHMQSVSSKQRVTGLDFIPGLHPVLSLSKMD
QTLAIYQQILTSLPSRNVIQISNDLENLRDLLHLLASSKSCPLPQARALETLESLGGVLEASHYSTEVVALSRLQGAL
QDMLRQLDLSPGC (SEQ ID NO: 108)
>gi|97071742|sp|Q1XG29.1|LEP_URSTH RecName: Full = Leptin; AltName:
Full = Obesity factor; Flags: Precursor
MRCGPLCRFLWLWPYLSYIEAVPIRKVQDDTKTLIKTIVTRINDISHTQAVSSKQRVAGLDFIPGLHPVLSLSRMDQ
TLAIYQQILTSLHSRNVVQISNDLENLRDLLHLLASSKSCPLPRARGLESFESLGGVLEASLYSTEVVALSRLQAAL
QDMLRRLDLSPGC (SEQ ID NO: 109)
>gi|20502048|gb|AAM21763.1| leptin [Vulpes lagopus]
SVSSKQRVAGLDFIPGLQPVLSLSKMDQTLAIYQQILTSLHSRNVVQISNDLENLRDLLHLLASSKSCPLPRARGLE
TFESLGGVLEASLYSTEVVALSRLQAALQDMLRRLDLSPGC (SEQ ID NO: 110)
>gi|20502052|gb|AAM21765.1| leptin [Vulpes vulpes]
SVSSKQRVAGLDFIPGLQPVLSLSKMDQTLAIYQQILTSLHSRNVVQISNDLENLRDLLHLLASSKSCPLPPARGLE
TFESLGGVLEASLYSTEVVALSRLQAALQDMLRRLDLSPGC (SEQ ID NO: 111)
>gi|114145273|emb|CAJ43200.2| Leptin [Zalophus californianus]
MRCGPLCQFLWLWPYLWYIEAVPIQKVQDDTKTLIKTIVTRINDISHTAVSSKQRVAGLDFIPGLHPVLSLSGMDQ
TLAIYQQILASLHSRNVGQISNDLENLRDLLHLLASSKTCPLPRARGLESFESLGSVLEGSLYSTEVVALSRLQAAL
QDMLWQLDLSPGC (SEQ ID NO: 112)

### REFERENCES

Athauda et al., (2003) Inhibition of human pepsin and gastricsin by alpha2-macroglobulin. J Enzyme Inhib Med Chem. 18(3): 219-24.
Cammisotto P. G., Renaud C., Gingras D., Delvin E., Levy E., Bendayan M. (2005). Endocrine and exocrine secretion of leptin by the gastric mucosa. J Histochem Cytochem 53: 851-60.
Cammisotto P G, Gingras D, Renaud C, Levy E, Bendayan M. (2006). Secretion of soluble leptin receptors by exocrine and endocrine cells of the gastric mucosa. Am J Physiol Gastrointest Liver Physiol 290: G242-9.
Cammisotto P. G., Gingras D., Bendayan M. (2007). Transcytosis of gastric leptin through the rat duodenal mucosa. Am J Physiol Gastrointest Liver Physiol 293: G773-9.
Cammisotto P G, Levy E, Bukowiecki L J, Bendayan M. Cross-talk between adipose and gastric leptins for the control of food intake and energy metabolism. (2010a) Prog Histochem Cytochem. 45:143-200.
Cammisotto P G, Bendayan M, Sané A, Dominguez M, Garofalo C and Levy E. Receptor-Mediated Transcytosis of Leptin Through Human Intestinal Cells In Vitro. (2010b). Int J Cell Biol. 2010:928169.
Campfield L. A., Smith F. J., Guisez Y., Devos R., Burn P. (1995). Recombinant mouse OB protein: evidence for a peripheral signal linking adiposity and central neural networks. Science. 269: 546-9.
Carpenter B., Pradhananga S., Wilkinson I., Justice S., Artymiuk P., Ross R. (2009). Expression and purification of a potential long-acting leptin agonist. Endocrine Abstracts. 19: P114.
Cinti S., Matteis R. D., Picó C., Ceresi E., Obrador A., Maffeis C., Oliver J, Palou A. (2000). Secretory granules of endocrine and chief cells of human stomach mucosa contain leptin. Int J Obes Relat Metab Disord. 24: 789-93.
Grasso S., Rozhayskaya-Arena M., Leinung M. C., Lee D. (2001). [D-LEU-4]-OB3, a synthetic leptin agonist, improves hyperglycemic control in C57B L/6/J ob/ob mice. Regulatory Peptides. 101: 123-129.
Guilmeau S., Buyse M., Tsocas A., Laigneau J. P., Bado A. (2003). Duodenal leptin stimulates cholecystokinin secretion: evidence of a positive leptin-cholecystokinin feedback loop. Diabetes. 52: 1664-72.
Hanew K. The effects of human leptin fragment (126-140) on pituitary functions in man. Eur J Endocrinol. 2003 November; 149(5):407-12.
Lo K M, Zhang J, Sun Y, Morelli B, Lan Y, Lauder S, Brunkhorst B, Webster G, Hallakou-Bozec S, Doaré L, Gillies S D. (2004). Engineering a pharmacologically superior form of leptin for the treatment of obesity. Protein Eng. Des. Sel. 18(1): 1-10.
Markowska A, Neri G, Hochol A, Nowak M, Nussdorfer G G, Malendowicz L K. (2004). Effects of leptin and leptin fragments on steroid secretion and proliferative activity of regenerating rat adrenal cortex. Int J Mol Med.
13(1):139-41.
Markowska A, Belloni A S, Rucinski M, Parenti A R, Nardelli G B, Drews K, Nussdorfer G G, Malendowicz L K. Leptin and leptin receptor expression in the myometrium and uterine myomas: Is leptin involved in tumor development? (2005). Int J Oncol. 27(6):1505-9.
Malendowicz L K, Neri G, Markowska A, Hochol A, Nussdorfer G G, Majchrzak M. (2003). Effects of leptin and leptin fragments on steroid secretion of freshly dispersed rat adrenocortical cells. J Steroid Biochem Mol Biol. 87(4-5):265-8.
Malendowicz L K, et al. (2004a). Acute in vivo effects of leptin and leptin fragments on corticosteroid hormone secretion and entero-insular axis in the rat. Int. J Mol. Med. 13(6): 829-834.
Malendowicz L K, Spinazzi R, Tortorella C, Nussdorfer G G, Ziolkowska A, Rucinski M. (2004b). Effects of leptin and leptin fragments on corticosterone secretion and growth of cultured rat adrenocortical cells. Int J Mol Med. 14(5):873-7.
Oliveira V X Jr, Fázio M A, Miranda M T, da Silva J M, Bittencourt J C, Elias C F, Miranda A. (2005). Leptin fragments induce Fos immunoreactivity in rat hypothalamus. Regul Pept. 127(1-3):123-32.
Sobhani I., Bado A., Vissuzaine C., Buyse M., Kermorgant S., Laigneau J. P., Attoub S., Lehy T., Henin D., Mignon M., Lewin M. J. (2000). Leptin secretion and leptin receptor in the human stomach. Gut 47: 178-83.
Solomon G., Niv-Spector L., Gonen-Berger D., Callebaut I., Djiane J., Gertler A. (2006). Preparation of Leptin Antagonists by Site-Directed Mutagenesis of Human, Ovine, Rat, and Mouse Leptin's Site III Implications on Blocking Undesired Leptin Action In Vivo. Ann. N.Y. Acad. Sci. 1091: 531-539.
Zhang Y., Proenca R., Maffei M., Barone M., Leopold L., Friedman J. M. (1994). Positional cloning of the mouse obese gene and its human homologue. Nature 372: 425-432.

1.-69. (canceled)

70. An oral composition comprising:
a) at least one leptin or a leptin functional derivative; and
b) at least one a pancreatic protease inhibitor.

71. The composition of claim 70, wherein the leptin or functional derivative thereof is a mammalian leptin.

72. The composition of claim 71, wherein the mammalian leptin is a human leptin or a dog leptin.

73. The composition of claim 70, wherein the pancreatic protease inhibitor comprises at least one trypsin inhibitor, chymotrypsin inhibitor, carboxypeptidase inhibitor and elastase inhibitor.

74. The composition of claim 70, wherein the pancreatic protease inhibitor is aprotinin.

75. The composition of claim 70, further comprising a stomach acid neutralizing agent.

76. The composition of claim 75, wherein the stomach acid neutralizing agent comprises at least one phosphate buffer, bicarbonate buffer, citrate buffer and acetate buffer.

77. The composition of claim 70, further comprising a bile acid or a bile acid analog.

78. The composition of claim 77, wherein the bile acid or bile acid analog comprises at least one deoxycholic acid, cholic acid, chenodeoxycholic acid, taurocholic acid, taurochenodeoxycholic acid, glycocholic acid, glycochenocholic acid, 3β-monohydroxychloric acid, lithocholic acid, 3-hydroxy-12-ketocholic acid, 12-3-dihydrocholic acid, ursodesoxycholic acid, or an analog of any of these.

79. The composition of claim 70, further comprising a stimulator of endogenous leptin secretion or a satiety triggering agent.

80. The composition of claim 79, wherein the stimulator of leptin secretion or satiety triggering agent comprises at least one amino acid, glutamine, insulin, secretin, cholecystokinin (CCK), pentagastrin, glucocorticoid, transretinoic acid, or an analog of any of these.

81. The composition of claim 70, further comprising a coating.

82. The composition of claim 81, wherein the coating is an enteric coating.

83. The composition of claim 70, further defined as a tablet, a pill, a powder, a syrup, a liquid, a food, a dragee, a confectionary, or any combination thereof.

84. A method for the oral administration of leptin, comprising administering to a subject a therapeutically effective amount of the oral composition of claim 70, wherein the leptin or leptin functional derivative is delivered to the subject's bloodstream in an active form.

85. The method of claim 84, wherein the administration of the leptin or leptin functional derivative treats at least one of obesity, type 1 diabetes, type 2 diabetes, hypothalamic amenorrhea, cardiovascular diseases, depression, a hypoleptinemic disease, and weight gain.


SEQUENCE LISTING (116 sequences)

SEQ ID NO: 1 - protein, 147 aa, Artificial Sequence
  Synthetic Peptide
MVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 2 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 3 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 4 - protein, 167 aa, Mus musculus
MCWRPLCRFLWLWSYLSYVQAVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 5 - protein, 146 aa, Mus musculus
VPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 6 - protein, 35 aa, Artificial Sequence
  Synthetic Peptide
VPIQKVQDDTKTLIKTIVTRINDISHTQSVSSKQK

SEQ ID NO: 7 - protein, 15 aa, Artificial Sequence
  Synthetic Peptide
YKVQDDTKTLIKTIV

SEQ ID NO: 8 - protein, 13 aa, Artificial Sequence
  Synthetic Peptide
NVIQISNDLENLR

SEQ ID NO: 9 - protein, 15 aa, Artificial Sequence
  Synthetic Peptide
ETLDSLGGVLEASGY

SEQ ID NO: 10 - protein, 30 aa, Artificial Sequence
  Synthetic Peptide
SGYSTEVVALSRLQGSLQDMLWQLDLSPGC

SEQ ID NO: 11 - protein, 18 aa, Artificial Sequence
  Synthetic Peptide
LQGSLQDMLWQLDLSPGC

SEQ ID NO: 12 - protein, 7 aa, Artificial Sequence
  Synthetic Peptide
SCHLPWA

SEQ ID NO: 13 - protein, 167 aa, Ailuropoda melanoleuca
MRCGPLCRFLWLWPYLSYIEAVPIRKVQDDTKTLIKTIVTRINDISHTQAVSSKQRVAGLDFIPGLHPVLSLSRMDQTLAIYQQILTSLHSRNVVQISNDLENLRDLLHLLASSKSCPLPRARGLESFESLGGVLEASLYSTEVVALSRLQAALQDMLQRLDLSPGC

SEQ ID NO: 14 - protein, 166 aa, Ailuropoda melanoleuca
MRCGPLCRFLWLWPYLSYIEAVPIRKVQDDTKTLIKTIVTRINDISHTAVSSKQRVAGLDFIPGLHPVLSLSRMDQTLAIYQQILTSLHSRNVVQISNDLENLRDLLHLLASSKSCPLPRARGLESFESLGGVLEASLYSTEVVALSRLQAALQDMLQRLDLSPGC

SEQ ID NO: 15 - protein, 147 aa, Anas platyrhynchos
VPIQKVQDDTKTLIKTIVTRINDISHTQSVSAKQRVTGLDFIPGLHPILSLSKMDQTLAVYQQVLTSLPSQNVLQIADDLENLRDLLHLLAFSKSCSLPQTSGLQKPESLDGVLEASLYSTEEVVALSRLQGSLQDILQQLDVSPEC

SEQ ID NO: 16 - protein, 146 aa, Anguilla japonica
GPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 17 - protein, 167 aa, Bos frontalis
MRCGPLYRFLWLWPYLSYVEAVPISKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 18 - protein, 97 aa, Bos frontalis
QSVSSKQRVTGLDFIPGLHPLLSLSKMDQTLAIYQQILTSLPSRNVVQISNDLENLRDLLHLLAASKSCPLPQVRALESLESLGVVLEASLYSTEVV

SEQ ID NO: 19 - protein, 167 aa, Bos grunniens
MRCGSLYRFLWLWPYLSYVEAVPISKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 20 - protein, 111 aa, Bos indicus
QSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLR

SEQ ID NO: 21 - protein, 116 aa, Bos indicus
SSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 22 - protein, 109 aa, Bos indicus
GLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 23 - protein, 146 aa, Bos taurus
VPIQKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPEC

SEQ ID NO: 24 - protein, 167 aa, Bos taurus
MRCGPLYRFLWLWPYLSYVEAVPIRKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 25 - protein, 100 aa, Bos taurus
SHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVV

SEQ ID NO: 26 - protein, 146 aa, Bos taurus
VPIRKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 27 - protein, 167 aa, Bos taurus
MRCGPLYRFLWLWPYLSYVEAVPICKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 28 - protein, 114 aa, Bos taurus x Bos indicus
KQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 29 - protein, 119 aa, Bubalus bubalis
QSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 30 - protein, 109 aa, Bubalus bubalis
QSVSSKQRVTGLDFIPGLHPLLSLSKMDQTLAIYQQILTSLPSRNVVQISNDLENLRDLLHLLAASKSCPLPQVRALESLKSLGVVLEASLYSTEVVALSRLQGSLQDM

SEQ ID NO: 31 - protein, 167 aa, Bubalus bubalis
MRCGPLYQFLWLWPYLSYVEAVPIRKVQDDTKTLIKTIVTRINDISHTQSVSSKQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 32 - protein, 108 aa, Bubalus bubalis
KQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQL

SEQ ID NO: 33 - protein, 108 aa, Bubalus bubalis
KQRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLQQL

SEQ ID NO: 34 - protein, 113 aa, Bubalus bubalis
QRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLRQLDLSPGC

SEQ ID NO: 35 - protein, 113 aa, Bubalus bubalis
QRVTGLDFIPGLHPLLSLSKMDQTLAIYQQILTSLPSRNVVQISNDLENLRDLLHLLAASKSCPLPQVRALESLESLGVVLEASLYSTEVVALSRLQGSLQDMLQQLDLSPGC

SEQ ID NO: 36 - protein, 111 aa, Bubalus bubalis
QSVSSKQRVTGLDFIPGLHPLLGLSKMDQTLAIYQQILTSLPSRNVVQISNDLENLRDLLHLLAASKSCPLPQVRALESLESLGVVLEASLYSTEVVALSRLQGSLQDMLR

SEQ ID NO: 37 - protein, 167 aa, Callithrix jacchus
MRWGCLCRFLWLWACLSYTQAVPIQRVQDDTKTLIKTIIARINDLSHTQSVSPRQRVTGLEFIPGFHSDLSFSKMDEILATYQQIVISLPSGNMIQISNDLENLRALLHLLAASKSCHLPWASGLENLANLGGVLEVSLYSTEVVALSRLRGTLKDILQQLDLGPAC

SEQ ID NO: 38 - protein, 109 aa, Camelus dromedarius
QSVSSKQRVTGLDFIPGLHPLLSLSKMDQTLAIYQQILTSLPSRNVVQISNDLESLRDLLHLLAASKSCPLPQVRALESLESLGVVLEASLYSTEVVALSWLQGSLQDM

SEQ ID NO: 39 - protein, 146 aa, Canis lupus familiaris
VPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALSRLQAALQDMLRRLDLSPGC

SEQ ID NO: 40 - protein, 118 aa, Canis lupus familiaris
SVSSKQRVAGLDFIPGLQPVLSLSRMDQTLAIYQQILNSLHSRNVVQISNDLENLRDLLHLLASSKSCPLPRARGLETFESLGGVLEASLYSTEVVALSRLQAALQDMLRRLDLSPGC

SEQ ID NO: 41 - protein, 167 aa, Canis lupus familiaris
MRCGPLCRFLWLWPYLSCVEAVPIRKVQDDTKTLIKTIVARINDISHTQSVSSKQRVAGLDFIPGLQPVLSLSRMDQTLAIYQQILNSLHSRNVVQISNDLENLRDLLHLLASSKSCPLPRARGLETFESLGGVLEASLYSTEVVALNRLQAALQDMLRRLDLSPGC

SEQ ID NO: 42 - protein, 106 aa, Capra hircus
  misc_feature (5)..(5): Xaa can be any naturally occurring amino acid
PGLHXVLSLSKMDQTLAIYQQILTSLPSRNVIQISNDLENLRDLLHLLASSKSCPLPQARALETLEXLGGVLEASLYSTEVVALTRLKGAFXDMLRKLDLALVVEA

SEQ ID NO: 43 - protein, 167 aa, Capra hircus
MRCGPLYRFLWLWPYLSYVEAVPIRKVQDDTKTLIKTIVTRINDISHTQSVSSKQRVTGLDFIPGLHPLLSLSKMDQTLAIYQQILASLPSRNVIQISNDLENLRDLLHLLAASKSCPLPQVRALESLESLGVVLEASLYSTEVVALSRLQGSLQDMLRQLDLSPGC

SEQ ID NO: 44 - protein, 147 aa, Capra hircus
MVPIRKVQDDTKTLIKTIVTRINDISHTQSVSSKQRVTGLDFIPGLHPLLSLSKMDQTLAIYQQILASLPSRNVIQISNDLENLRDLLHLLAASKSCPLPQVRALESLESLGVVLEASLYSTEVVALSRLQGSLQDMLRQLDLSPGC

SEQ ID NO: 45 - protein, 146 aa, Channa argus
  misc_feature (131)..(131): Xaa can be any naturally occurring amino acid
VPIQEVQDDTKTLIKTIVTRINDISHTQSVSAKQRVTGLDFIPGLHPILSLSKMDQTLAVYQQVLTSLPSQNVLQIANDLKNLRDLLHLLAFSKSCSLPQTSGLQKPESLDGVLEASLYSTEVVALSRLQXSLQDILQQLDVSPEC

SEQ ID NO: 46 - protein, 146 aa, Ctenopharyngodon idella; identical to SEQ ID NO: 5

SEQ ID NO: 47 - protein, 146 aa, Culter Sp. TP-2004
VPIQKVQDDSKTLIKTIVTRINDISHTQSVSAKQRVTGLDFIPGLHPILSLSKMDQTLAVYQQVLTSLPSQNVLQIANGLKNLRDLLHLLAFSKSCSLPQTSGLQKPESLDGVLEASLYSTEVVALSRLQGSLQDILQQLDVSPEC

SEQ ID NO: 48 - protein, 146 aa, Cyprinus carpio
VPIQKVQDDTKTLIKTIVTRINDISHTQSVSAKQRVTGLDFIPGLHPILSLSKMDQTLAVYQQVLTSLPSQNVLQIANDLKNLRDLLHLLAFSKSCSLPQTSGLQKPESLDGVLEASLYSTEVVALSRLQGSLQDILQQLDVSPEC

SEQ ID NO: 49 - protein, 99 aa, Equus caballus
DTKTLIKTIVTRINDISHTQSVSSKQRVTGLDFIPGLHPVLSLSKMDQTLAIYQQILTSLPSRNVIQISNDLENLRDLLHLLASSKSCPLPQARGLETL

SEQ ID NO: 50 - protein, 145 aa, Equus caballus
LWLWPYLFFIEAVPIRKVQDDTKTLIKTIVTRINDISHTQSVSSKQRVTGLDFIPGLHPVLSLSKMDQTLAIYQQILTSLPSRNVIQISNDLENLRDLLHLLASSKSCPLPQARGLETLASLGGVLEASLLLHRGGSPEQAAGVS

SEQ ID NO: 51 - protein, 167 aa, Equus caballus
MHCGPLCQFLWLWPYLFFIEAVPIRKVQDDTKTLIKTIVTRINDISHTQSVSSKQRVTGLDFIPGLHPVLSLSKMDQTLAIYQQILTSLPSRNVIQISNDLENLRDLLHLLASSKSCPLPQARGLETLASLGGVLEASLYSTEVVALSRLQGSLQDMLQQLDLSPGC

SEQ ID NO: 52 - protein, 167 aa, Felis catus
MLCGPLCRFLWLWPYLSYVEAVPIRKVQDDTKTLIKTIVTRINDISHTQSVSSKQRVAGLDFIPGLHPVLSLSKMDQTLAIYQQILTGLPSRNVVQISNDLENLRDLLHLLASSKNCPLPRARGLETLESLGGALEASLYSTEVVALSRLQASLQDMLWRLDLSPGC

SEQ ID NO: 53 - protein, 163 aa, Gallus gallus
MCWRPLCRLWSYLVYVQAVPCQIFQDDTKTLIKTIVTRINDISHTSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDISPEC

SEQ ID NO: 54 - protein, 146 aa, Gorilla gorilla
VPIQKVQDDTKTLIKTIVTRISDISHTQSVSSKQKVTGLDFIPGLHPILTLSKMDQTLAVYQQILTSMPSRNMIQISNDLENLRDLLHVLAFSKSCHLPWASGLETLDSLGGVLEASGYSTEVVALSRLQGSLQDMLWQLDLSPGC

SEQ ID NO: 55 - protein, 167 aa, Halichoerus grypus
MRCGSLCRFLWLWSCLPYIEAMPIQRVQDDTKTLIKTIITRINDISPPQGVCSRPRVAGLDFIPRVQSVRTLSGMDQILATYQQILTSLQSRSVVQIANDLANLRALLRLLASAKSCPVPRARGSDTIKGLGNVLRASVHSTEVVALSRLKAALQDMLRQLDRNPGC

SEQ ID NO: 56 - protein, 166 aa, Homo sapiens
MHWGTLCGFLWLWPYLFYVQAVPIQKVQDDTKTLIKTIVTRINDISHTSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 57 - protein, 167 aa, Homo sapiens [sequence omitted]

SEQ ID NO: 58 - protein, 167 aa, Homo sapiens; identical to SEQ ID NO: 2

SEQ ID NO: 59 - protein, 167 aa, Homo sapiens [sequence omitted]

SEQ ID NO: 60 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPEASGLETLDSLGGVLEASGYSTEVVALSRLQGSLQDMLWQLDLSPGC

SEQ ID NO: 61 - protein, 167 aa, Homo sapiens [sequence omitted]

SEQ ID NO: 62 - protein, 146 aa, Hypophthalmichthys nobilis
  misc_feature (131)..(131): Xaa can be any naturally occurring amino acid
VPIQKVQDDSKTLIKTIVTRINDISHTQSVSAKQRVTGLDFIPGLHPILSLSKMDQTLAVYQQVLTSLPSQNVLQIANGLKNLRDLLHLLAFSKSCSLPQTSGLQKPESLDGVLEASLYSTEVVALSRLQXSLQDILQQLDVSPEC

SEQ ID NO: 63 - protein, 122 aa, Lagenorhynchus albirostris
VQDDTKTLIKTIVTRINDISHTRSVSSKQRVTGLDFIPGLTPVLSLSKMDQTLTIYQQILTSLPSRNVIQISNDLENLRDLLHLLASSKSCPLPQARALETLESLGGVLEASLYSTEVVAQS

SEQ ID NO: 64 - protein, 140 aa, Leptonychotes weddelli
VQDDTKTLIRTIIARINDISQPGVCSRPRVAGLDFIPGPQSVRTLSGMNQMLAIYQQILTSLHSRSVVQIANDLANLRDLLHLLASAKSCPLPRARGLENIKSLRDVLKASVHSTEVVALSRLRAALQGMLRQLDRNPGC

SEQ ID NO: 65 - protein, 167 aa, Lepus oiostolus
MRCGPLCRLLWLWPCLSCVPAVPMRKVQDDTKTLIKTIVTRISDISHTQSVSSRQRVVGLDFIPGLHPNLSLSTMDQTLAIYQQILTSLPSRNVIQIANDLENLRDLLHLLALSKSCPLPRASGLETLEGLGGVLEASLYSTEVVALSRLQGSLQAMLQQLDLGPGC

SEQ ID NO: 66 - protein, 167 aa, Macaca mulatta
MYWRTLWGFLWLWPYLFYIQAVPIQKVQSDTKTLIKTIVTRINDISHTQSVSSKQRVTGLDFIPGLHPVLTLSQMDQTLAIYQQILINLPSRNVIQISNDLENLRDLLHLLAFSKSCHLPLASGLETLESLGDVLEASLYSTEVVALSRLQGSLQDMLWQLDLSPGC

SEQ ID NO: 67 - protein, 146 aa, Megalobrama amblycephala
VPIQKVQDDTKTLIKTIVTRINDISHTQSVSAKQRVTGLDFIPGLHPILSLSKMDQTLAVYQQVVTSLPSQNVLQIANDLKNLRDLLHLLAFSKSCSLPQTSGLQKPESLDGVLEASLYSTEVVALSRLQGSLQDILQQLDVSPEC

SEQ ID NO: 68 - protein, 145 aa, Meleagris gallopavo
VPCQIFQDDTKTLIKTIVTRINDISHTSVSAKQSVTGLDFIPGLHPILSLSKMDQTLAVYQQVLTSLPSQNVLQIANDLENLRDLLHLLAFSKSCSLPQTSGLHKPESLDGVLEALLYSTEVVALSRLQGSLQDILQQLDISPEC

SEQ ID NO: 69 - protein, 167 aa, Monodelphis domestica
MHCVALCSFLWLCHHLYYTQAVPIRKVQDDTKTLTKTIITRINDISHMYSISAKQRVTGLDFIPGLHPFQSLSDMDQTLAIYQQILSNLSSRNMVQISNDLENLRDLLHLLGSLKSCPFDEADGLSSLGNLEGVMEASLYSTEVVTLTRLQKSLYGMLQQLDLIHGC

SEQ ID NO: 70 - protein, 167 aa, Mus musculus; identical to SEQ ID NO: 4

SEQ ID NO: 71 - protein, 123 aa, Myotis lucifugus
FYAEAAPIQKVQDDTKTLIKTIVTRINDISHTRSVSSRQRVTGLDFIPGLHPILSLSRMDQTLAIYQQILTSLPSGNVLQISNDLENLRDLLHLLASSNSCPFPRTRSLKTLEGLDDALEASL

SEQ ID NO: 72 - protein, 166 aa, Neovison vison
MLCGPLCRFLWLWPYLSYVEAVPIRKVQDDTKTLIKTIVTRISDISHTAVSSKQRVAGLDFIPGLHPVLSLSRMDQTLAIYQQILTSLHSRNVIQISNDLENLRDLLRLLASSKSCPLPRARGLESFESLGGVLEASLYSTEVVALSRLQAALQDMLGRLDLSPGC

SEQ ID NO: 73 - protein, 167 aa, Notomys alexis
MCWRPLCWFLWLWSYLSYVQALPVQKVQDDTKTLIKTIATRINDISHTQSVSAKQRVTGLDFIPGLHPILSLSKMDQTLVVYQQILTSLPSGNVLQIANDLENLRDLLRLLAFSKSCSLPQTSGLQKPESLDGVLEASLYSTEVVALSRLQGFLQDILQQLDLTPEC

SEQ ID NO: 74 - protein, 118 aa, Nyctereutes procyonoides
SVSSKQRVAGLDFIPGLQPVLSLSRMDQTLAIYQQILTSLHSRNVVQISNDLENLRDLLHLLASSKSCPLPRARGLETFESLGGVLEASLYSTEVVALSRLQAALQDMLRRLDLSPGC

SEQ ID NO: 75 - protein, 167 aa, Ochotona cansus
MRCGPLRQLLWLWPCLLCVQAVSIWKVRDDTKTLIKTIVIRISDISHTHAVSSKQRITGLDFIPALHPNLSLSKMDQTLVLYKHILTSLPSRNVVQIANDLENLRDLLHLLAASQGCPPPRASDLESLNSLESILEASLYSTEVVALSRLQGSLHEMLQQLDIGPGC

SEQ ID NO: 76 - protein, 167 aa, Ochotona curzoniae
MRCGPLCQLLWLWPCLLCVQAVSIWKVRDDTKTLIKTILTRISDISHTHAVSSKQRITGLDFIPALHPNLSLSKMDQTLVLYKHILTSLPSRNVVQIANDLENLRDLLHLVAASQGCPPPRASDLESLNSLESILEASLYSTEVVALSRLQGSLHEMLQQLDIGPGC

SEQ ID NO: 77 - protein, 167 aa, Ochotona dauurica
MRCGPLCQLLWLWPCLLCVQAVSIWKVRDDTKTLIKTIVTRISDISHTHAVSSKQRITGLDFIPALHPNLSLSKMDQTLVLYKHILTSLPSRNVVQIANDLENLRDLLHLLAASQGCPPPRASDLESLNSLESILEASLYSTEVVALSRLQGSLHEMLQQLDIGPGC

SEQ ID NO: 78 - protein, 167 aa, Ochotona dauurica bedfordi
MRCGPLCQLLWLWPCLLCVQAVSIWKVRDDTKTLIKTIVTRISDISHTHAVSSKQRITGLDLIPALHPNLSLSKMDQTLVLYKHILTSLPSRNVVQIANDLENLRDLLHLLAASQGCPPPRASDLESLNSLESILEASLYSTEVVALSRLQGSLHEMLQQLDIGPGC

SEQ ID NO: 79 - protein, 167 aa, Ochotona thomasi
MRCGPLCQLLWLWPCLLCVQAVSIWKVRDDTKTLIKTILTRISDISHTHAVSSKQRITGLDFTPALHPNLSLSKMDQTLVLYKHILTSLPSRNVVQIADDLENLRDLLHLVAASQGCPPPRASDLESLNSLESILEASLYSTEVVALSRLQGSLHEMLQQLDIGPGC

SEQ ID NO: 80 - protein, 167 aa, Oryctolagus cuniculus
MRCGPLCQLLWLWPCLSCVPAVPMRKVQDDTKTLIKTIVTRISDISHTQSVSSRQRVVGLDFIPGLHPNLSLSTMDQTLAIYQQILASLPSRNVIQIANDLENLRDLLHLLASSKSCPLPRASGLETLEGLGGVLEASLYSTEVVALSRLQGFLQAMLQQLDLGPGC

SEQ ID NO: 81 - protein, 117 aa, Ovis aries
DTKTLIKTIVTRINDISHTQSVSSKQRVTGLDFIPGLHPLLSLSKMDQTLAIYQQIHASLPSRNVIQISNDLENLRDLLHLLAGSKSCPLPQVRALESLESLGVVLEASLYSTEVLA

SEQ ID NO: 82 - protein, 146 aa, Ovis aries
VPIRKVQDDTKTLIKTIVTRINDISHTQSVSSKQRVTGLDFIPGLHPLLSLSKMDQTLAIYQQILASLPSRNVIQISNDLENLRDLLHLLAASKSCPLPQVRALESLESLGVVLEASLYSTEVVALSRLQGSLQDMLRQLDLSPGC

SEQ ID NO: 83 - protein, 110 aa, Ovis aries
TGLDFIPGLHPLLSLSKMDQTLAIYQQILASLPSRNVIQISNDLENLRDLLHLLAASKSCPLPQVRALESLESLGVVLEASLYSTEVVALSRLQGSLQDMLRQLDLSPGC

SEQ ID NO: 84 - protein, 119 aa, Ovis aries
QSVSSKQRVTGLDFIPGLHPLLSLSKMDQTLAIYQQILASLPSRNVIQISNDLENLRDLLHLLAASKSCPLPQVRALESLESLGVVLEASLYSTEVVALSRLQGSLQDMLRQLDLSPGC

SEQ ID NO: 85 - protein, 119 aa, Ovis aries
QSVSSKQRVTGLDFIPGLHPLLSLSKMDQTLAIYQQILASLPSRNVIQISNDLENLRDLLHLLAASKSCPLPQVRALESLESLGVVLEASLYSTELVALSRLQGSLQDMLRQLDLSPGC

SEQ ID NO: 86 - protein, 119 aa, Ovis aries
QSVSSKQRVTGLDFIPGLHPLLSLSKMDQTLAIYQQILASLPSRNVIQISNDLENLRDLLHLLAASKSCPLQQVRALESLESLGVVLEASLYSTEVVALSRLQGSLQDMLRQLDLSPGC

SEQ ID NO: 87 - protein, 119 aa, Ovis aries
QSVSSKQRVTGLDFIPGLHPLLSLSKMDQTLAIYQQILASLPSRNVIQISNDLENLQDLLHLLAASKSCPLPQVRALESLESLGVVLEASLYSTEVVALSRLQGSLQDMLRQLDLSPGC

SEQ ID NO: 88 - protein, 140 aa, Ovis aries
QDDTKTLIKTIVTRINDISHTQSVSSKQRVTGLDFIPGLHPLLSLSKMDQTLAIYQQILASLPSRNVIQISNDLENLRDLLHLLAASKSCPLPQVRALESLESLGVVLEASLYSTELVALSRLQGSLQDMLRQLDLSPGC

SEQ ID NO: 89 - protein, 140 aa, Ovis aries
QDDTKTLIKTIVTRINDISHTQSVSSKQRVTGLDFIPGLHPLLSLSKMDQTLAIYQQILASLPSRNVIQISSDLENLRDLLHLLAASKSCPLPQVRALESLESLGVVLEASLYSTEVVALSRLQGSLQDMLRQLDLSPGC

SEQ ID NO: 90 - protein, 139 aa, Ovis aries
QDDTKTLIKTIVTRINDISHTSVSSKQRVTGLDFIPGLHPLLSLSKMDQTLAIYQQILASLPSRNVIQISNDLENLRDLLHLLAASKSCPLPQVRALESLESLGVVLEASLYSTELVALSRLQGSLQDMLRQLDLSPGC

SEQ ID NO: 91 - protein, 140 aa, Ovis aries
QDDTKTLIKTIVTRINDISHTQSVSSKQRVTGLDFIPGLHPLLSLSKMDQTLAIYQQILASLPSRNVIQISNDLENLQDLLHLLAASKSCPLPQVRALESLESLGVVLEASLYSTEVVALSRLQGSLQDMLQQLDLSPGC

SEQ ID NO: 92 - protein, 146 aa, Pan troglodytes
VPIQKVQDDTKTLIKTIVTRINDISHTQSVSSKQKVTGLDFIPGLHPILTLSKMDQTLAVYQQILTSMPSRNMIQISNDLENLRDLLHVLAFSKSCHLPWASGLETLDSLGGVLEASGYSTEVVALSRLQGSLQDMLWQLDLSPGC

SEQ ID NO: 93 - protein, 167 aa, Pan troglodytes [sequence omitted]

SEQ ID NO: 94 - protein, 167 aa, Phoca vitulina
MRCGSLCRFLWLWSCLSYIEAVPIQRVQDDTKTLIKTIITRINDISPPQGVCSRPRVAGLDFIPRVQSVRTLSGMDQILATYQQILTSLQSRSVVQIANDLANLRALLRLLASAKSCPVPRARGSDTIKGLGNVLRASVHSAEVVALSRLKAALQDMLRQLDRNPGC

SEQ ID NO: 95 - protein, 138 aa, Phocoena phocoena
MRCGPLCRFLWLWPYLSYIEAVPIRKVQDDTKTLIKTIVTRINDISHTQSVSSKQRVTGLDFIPGLTPVLSLSKMDQTLAIYQQILTSLPSRNVIQISNDLENLRDLLHLLASSKSCPLPQARALETLESLGGVLEAS

SEQ ID NO: 96 - protein, 118 aa, Phodopus campbelli
VQDDTKTLIKTIVTRINDISHTQSVSAKQRVTGLDFIPGLHPILSLSKMDQTLAVYQQILTSLPSRNMVQISNDLENLRDLLHLLASSKSCSLPQTSELQKLESLDGVLEASLYSTEV

SEQ ID NO: 97 - protein, 167 aa, Pongo abelii
MHWGTLCGFLWLWPYLFYVQAVPIQKVQDDTKTLIKTVITRINDISHTQSVSSKQKVTGLDFIPGLHPILTLSKMDQTLAVYQQILTSMPSRNVIQISNDLENLRDLLHVLAFSKSCHLPWASGLETLDRLGGVLEASGYSTEVVALSRLQRSLQDMLWQLDLSPGC

SEQ ID NO: 98 - protein, 146 aa, Pongo pygmaeus
VPIQKVQDDTKTLIKTVITRINDISHTQSVSSKQKVTGLDFIPGLHPILTLSKMDQTLAVYQQILTSMPSRNVIQISNDLENLRDLLHVLAFSKSCHLPWASGLETLDRLGGVLEASGYSTEVVALSRLQRSLQDMLWQLDLSPGC

SEQ ID NO: 99 - protein, 154 aa, Rattus norvegicus
SYLSYVQAVPIHKVQDDTKTLIKTIVTRINDISHTQSVSARQRVTGLDFIPGLHPILSLSKMDQTLAVYQQILTSLPSQNVLQIAHDLENLRDLLHLLAFSKSCSLPQTRGLQKPESLDGVLEASLYSTEVVALSRLQGSLQDILQQLDVSPEC

SEQ ID NO: 100 - protein, 167 aa, Rattus norvegicus
MCWRPLCRFLWLWSYLSYVQAVPIHKVQDDTKTLIKTIVTRINDISHTQSVSARQRVTGLDFIPGLHPILSLSKMDQTLAVYQQILTSLPSQNVLQIAHDLENLRDLLHLLAFSKSCSLPQTRGLQKPESLDGVLEASLYSTEVVALSRLQGSLQDILQQLDLSPEC

SEQ ID NO: 101 - protein, 146 aa, Silurus asotus
VPIQKVQDDTKTLIKTIVTRINDISHTQSVSAKQTLTGLDFIPGLHPILSLSKMDQTLAVYQQVLTSLPSQNVLQIANDLKNLRDLLHLLAFSKSCSLPQTSGLQKPESLDGVLEASLYSTEVVALSRLQGSLQDILQQLDVSPEC

SEQ ID NO: 102 - protein, 167 aa, Sminthopsis crassicaudata
MHCVPLFCFLWFCHHLYYSQAVPIRKVQDDTKTLTKTIITRINDISHMYSISAKQRVTGLDFIPGLHPFQSLSDMDQTLAIYQQILSNLSSRNMVQISNDLENLRDLLHLLGSLKSCPFDEAGGLSALGNLEGVMEASLYSTEVVTLTRLQKSLYVMLQQLDLIHGC

SEQ ID NO: 103 - protein, 96 aa, Sus scrofa
YLSYVEGPPIWRVQDDTKTLIKTIVTRISDISHMQSVSSKQRVTGLDFIPGLHPVLSLSKMDQTLAIYQQILTSLPSRNVIQLSNDLENLRDLLHL

SEQ ID NO: 104 - protein, 167 aa, Sus scrofa
MRCGPLCRFLWLWPYLSYVEAVPIWRVQDDTKTLIKTIVTRISDISHMQSVSSKQRVTGLDFIPGLHPVLSLSKMDQTLAIYQQILTSLPSRNVIQISNDLENLRDLLHLLASSKSCPLPQRRALETLESLGGVLEASLYSTEVVALSRLQGALQDMLRQLDLSPGC

SEQ ID NO: 105 - protein, 167 aa, Sus scrofa
  misc_feature (64)..(64): Xaa can be any naturally occurring amino acid
MRCGPLCRFLWLWPYLSYVEAVPIWRVQDDTKTLIKTIVTRISDISHMQSVSSKQRVTGLDFIXGLHPVLSLSKMDQTLAIYQQILTSLPSRNVIQISNDLENLRDLLHLLASSKSCPLPQARALETLESLGGVLEASLYSTEVVALSRLQGALQDMLRQLDLSPGC

SEQ ID NO: 106 - protein, 167 aa, Sus scrofa
MRCGPLCRFLWLWPYLSYVEAVPIWRVQDDTKTLIKTIVTRISDISHMQSVSSKQRVTGLDFIPGLHPVLSLSKMDQTLAIYQQILTSLPSRNVIQISNDLENLRDLLHLLASSKSCPLPQARALETLESLGGVLEASLYSTEVVALSRLQGALQDMLRQLDLSPGC

SEQ ID NO: 107 - protein, 146 aa, Sus scrofa
VPIWRVQDDTKTLIKTIVTRISDISHMQSVSSKQRVTGLDFIPGLHPVLSLSKMDQTLAIYQQILTSLPSRNVIQISNDLENLRDLLHLLASSKSCPLPQARALETLESLGGVLEASLYSTEVVALSRLQGALQDMLRQLDLSPGC

SEQ ID NO: 108 - protein, 167 aa, Sus scrofa
MRCGPLCRFLWLWPYLSYVEAVPIWRVQDDTKTLIKTIVTRISDISHMQSVSSKQRVTGLDFIPGLHPVLSLSKMDQTLAIYQQILTSLPSRNVIQISNDLENLRDLLHLLASSKSCPLPQARALETLESLGGVLEASHYSTEVVALSRLQGALQDMLRQLDLSPGC

SEQ ID NO: 109 - protein, 167 aa, Ursus thibetanus
MRCGPLCRFLWLWPYLSYIEAVPIRKVQDDTKTLIKTIVTRINDISHTQAVSSKQRVAGLDFIPGLHPVLSLSRMDQTLAIYQQILTSLHSRNVVQISNDLENLRDLLHLLASSKSCPLPRARGLESFESLGGVLEASLYSTEVVALSRLQAALQDMLRRLDLSPGC

SEQ ID NO: 110 - protein, 118 aa, Vulpes lagopus
SVSSKQRVAGLDFIPGLQPVLSLSKMDQTLAIYQQILTSLHSRNVVQISNDLENLRDLLHLLASSKSCPLPRARGLETFESLGGVLEASLYSTEVVALSRLQAALQDMLRRLDLSPGC

SEQ ID NO: 111 - protein, 118 aa, Vulpes vulpes
SVSSKQRVAGLDFIPGLQPVLSLSKMDQTLAIYQQILTSLHSRNVVQISNDLENLRDLLHLLASSKSCPLPPARGLETFESLGGVLEASLYSTEVVALSRLQAALQDMLRRLDLSPGC

SEQ ID NO: 112 - protein, 166 aa, Zalophus californianus
MRCGPLCQFLWLWPYLWYIEAVPIQKVQDDTKTLIKTIVTRINDISHTAVSSKQRVAGLDFIPGLHPVLSLSGMDQTLAIYQQILASLHSRNVGQISNDLENLRDLLHLLASSKTCPLPRARGLESFESLGSVLEGSLYSTEVVALSRLQAALQDMLWQLDLSPGC

SEQ ID NO: 113 - protein, 147 aa, Artificial Sequence
  Synthetic Peptide
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 114 - protein, 147 aa, Artificial Sequence
  Synthetic Peptide
MVPIHKVQDDTKTLIKTIVTRINDISHTQSVSARQRVTGLDFIPGLHPILSLSKMDQTLAVYQQILTSLPSQNVLQIAHDLENLRDLLHLLAFSKSCSLPQTRGLQKPESLDGVLEASLYSTEVVALSRLQGSLQDILQQLDLSPEC

SEQ ID NO: 115 - protein, 146 aa, Artificial Sequence
  Synthetic Peptide
VPIXKVQDDTKTLIKTIVTRINDISHTQSVSXXQXVTGLDFIPGLHPILXLSKMDQTLAVYQQXLTSXPSXNVXQIXXDLENLRDLLHXLAFSKSCXLPXXXGLXXXXSLXGVLEASXYSTEVVALSRLQGSLQDXLXQLDXSPXC

SEQ ID NO: 116 - protein, 167 aa, Artificial Sequence
  Synthetic Peptide
MHWGTLCGFLWLWPYPFYVQAVPIQKVQDDTKTLIKTIVTRINDISHTXSVSSKQKVTGLDFIPGLHPILTLSKMXQTLAVYQQILTSMPSRNVXQISNDLENLRDLLHVLAFSKSCHLPXASGLETLDSLGGVLEASGYSTEVVALSRLQGSLQDMLWQLDLSPGC